(19) United States
(12) Patent Application Publication
(10) Pub. No.: US 2026/0151023 A1
(43) Pub. Date: Jun. 4, 2026

(54) MEDICAL STEREOSCOPIC OBSERVATION IMAGING DEVICE, MEDICAL STEREOSCOPIC OBSERVATION SYSTEM, AND MEDICAL IMAGE PROCESSING DEVICE

(71) Applicant: Sony Olympus Medical Solutions Inc., Tokyo (JP)

(72) Inventors: Hiroshi USHIRODA, Tokyo (JP); Takahiro YAMAMOTO, Tokyo (JP)

(73) Assignee: Sony Olympus Medical Solutions Inc., Tokyo (JP)

(21) Appl. No.: 19/375,248

(22) Filed: Oct. 31, 2025

(30) Foreign Application Priority Data
Dec. 3, 2024 (JP) 2024-210137

(51) Int. Cl. A61B 1/04; A61B 1/00; A61B 1/05; A61B 1/06; H04N 13/243; H04N 13/254; H04N 13/361; H04N 23/13

(52) U.S. Cl. CPC A61B 1/043; A61B 1/00009; A61B 1/00048; A61B 1/00193; A61B 1/05; A61B 1/0638; H04N 13/243; H04N 13/254; H04N 13/361; H04N 23/13

(57) ABSTRACT

A medical stereoscopic observation imaging device includes: a first imaging unit configured to capture each of first normal observation light and at least one of first fluorescence or second fluorescence; and a second imaging unit configured to capture each of second normal observation light and at least an other one of the first fluorescence or the second fluorescence. The first normal observation light and the second normal observation light are components of return light from an observation target with which normal light including at least a part of a wavelength band of visible light is irradiated and are components of observation light having parallax with each other, and the first fluorescence and the second fluorescence are components of fluorescence emitted from the observation target and have different wavelength bands.

### CROSS-REFERENCE TO RELATED APPLICATION

This application claims priority from Japanese Application No. 2024-210137, filed on Dec. 3, 2024, the contents of which are incorporated by reference herein in its entirety.

### BACKGROUND

The present disclosure relates to a medical stereoscopic observation imaging device, a medical stereoscopic observation system, and a medical image processing device.

In the related art, there is known a medical stereoscopic observation system that irradiates an observation target (subject such as a person) with light from a light source device and captures return light from the observation target to enable stereoscopic observation of the observation target (for example, JP 2021-145873 A).

The medical stereoscopic observation system described in JP 2021-145873 A includes right-eye and left-eye imaging units that capture observation light components for right and left eyes having parallax with each other. The observation light for right eye includes: return light from an observation target with which normal light including at least a part of a wavelength band of visible light is irradiated (hereinafter, referred to as return light of normal light); and fluorescence emitted from a substance in the observation target by irradiation of excitation light that is narrow-band light. The right-eye imaging unit includes two image sensors including an image sensor that captures the return light of the normal light and an image sensor that captures the fluorescence. The same applies to the observation light for left eye and the left-eye imaging unit as in the observation light for right eye and the right-eye imaging unit. That is, the left-eye imaging unit includes two image sensors as in the right-eye imaging unit.

### SUMMARY

Incidentally, there are needs for observing not only one type of fluorescence but also two types of fluorescence having different wavelength bands. In the medical observation system described in JP 2021-145873 A, one type of fluorescence can be observed, but two types of fluorescence having different wavelength bands (hereinafter, referred to as first fluorescence and second fluorescence) cannot be observed.

Here, when the observation of the return light of the normal light (hereinafter, referred to as normal light observation), the observation of the first fluorescence (hereinafter, referred to as first fluorescence observation), and the observation of the second fluorescence (hereinafter, referred to as second fluorescence observation) are executed, achievement of the performance of the first fluorescence observation and achievement of the performance of the second fluorescence observation are necessary. As the intensity of the fluorescence becomes weaker, the degree of difficulty becomes higher, and it is necessary to avoid circumstances where the first and second fluorescence observation are inhibited by the return light of the normal light in the normal light observation, the second fluorescence observation is inhibited by first excitation light for emitting the first fluorescence, and the first fluorescence observation is inhibited by second excitation light for emitting the second fluorescence. To that end, the separability of wavelengths of light needs to be improved.

In order to improve the separability of wavelengths of light, a configuration including not only an optical system dedicated to the first and second fluorescence observation but also three image sensors including an image sensor that captures the return light of the normal light, an image sensor that captures the first fluorescence, and an image sensor that captures the second fluorescence is assumed. However, in the case of the medical stereoscopic observation system that enables stereoscopic vision of the observation target, the above-described configuration is necessary in the right-eye and left-eye imaging units, and miniaturization cannot be achieved.

Accordingly, a technique capable of achieving miniaturization while enabling the return light of the normal light and two types of fluorescence to be captured has been demanded.

According to one aspect of the present disclosure, there is provided a medical stereoscopic observation imaging device including: a first imaging unit configured to capture each of first normal observation light and at least one of first fluorescence or second fluorescence; and a second imaging unit configured to capture each of second normal observation light and at least an other one of the first fluorescence or the second fluorescence, wherein the first normal observation light and the second normal observation light are components of return light from an observation target with which normal light including at least a part of a wavelength band of visible light is irradiated and are components of observation light having parallax with each other, and the first fluorescence and the second fluorescence are components of fluorescence emitted from the observation target and have different wavelength bands.

According to another aspect of the present disclosure, there is provided a medical stereoscopic observation system including: a medical stereoscopic observation imaging device including a first imaging unit configured to capture each of first normal observation light and at least one of first fluorescence or second fluorescence, and a second imaging unit configured to capture each of second normal observation light and at least the other one of the first fluorescence and the second fluorescence; and a medical image processing device configured to process a captured image obtained by capturing of the medical stereoscopic observation imaging device, wherein the first normal observation light and the second normal observation light are components of return light from an observation target with which normal light including at least a part of a wavelength band of visible light is irradiated and are components of observation light having parallax with each other, and the first fluorescence and the second fluorescence are components of fluorescence emitted from the observation target and have different wavelength bands.

According to still another aspect of the present disclosure, there is provided a medical image processing device including a processor configured to process a captured image obtained by capturing of a medical stereoscopic observation imaging device, wherein the medical stereoscopic observation imaging device includes: a first imaging unit configured to capture each of first normal observation light and at least one of first fluorescence or second fluorescence; and a second imaging unit configured to capture each of second normal observation light and at least the other one of the first fluorescence and the second fluorescence, the first normal observation light and the second normal observation light are components of return light from an observation target with which normal light including at least a part of a wavelength band of visible light is irradiated and are components of observation light having parallax with each other, and the first fluorescence and the second fluorescence are components of fluorescence emitted from the observation target and have different wavelength bands.

### BRIEF DESCRIPTION OF THE DRAWINGS

FIG. 1 is a diagram illustrating a configuration of a medical stereoscopic observation system 1 according to an embodiment;

FIG. 2 is a block diagram illustrating a configuration of a camera head and a control device;

FIG. 3 is a diagram illustrating a configuration of first and second imaging units;

FIG. 4 is a diagram illustrating another example of a configuration of the first and second imaging units;

FIG. 5 is a diagram illustrating another example of a configuration of the first and second imaging units;

FIG. 6 is a diagram illustrating operations of the first imaging unit and a light source device in a first mode;

FIG. 7 is a diagram illustrating operations of the second imaging unit and the light source device in the first mode;

FIG. 8 is a diagram illustrating an updated image of a captured image generated in the first mode;

FIG. 9 is a diagram illustrating operations of the first imaging unit and the light source device in second and third modes;

FIG. 10 is a diagram illustrating operations of the second imaging unit and the light source device in the second and third modes;

FIG. 11 is a diagram illustrating an updated image of a captured image generated in the second and third modes;

FIG. 12 is a diagram illustrating operations of the first imaging unit and the light source device in a fourth mode;

FIG. 13 is a diagram illustrating operations of the second imaging unit and the light source device in the fourth mode;

FIG. 14 is a diagram illustrating an updated image of a captured image generated in the fourth mode;

FIG. 15 is a diagram illustrating a configuration of the first and second imaging units in Modification Example 1 of the embodiment;

FIG. 16 is a diagram illustrating operations of the first imaging unit and the light source device in the third mode;

FIG. 17 is a diagram illustrating operations of the second imaging unit and the light source device in the third mode;

FIG. 18 is a diagram illustrating an updated image of a captured image generated in the third mode;

FIG. 19 is a diagram illustrating operations of the first imaging unit and the light source device in the fourth mode;

FIG. 20 is a diagram illustrating operations of the second imaging unit and the light source device in the fourth mode;

FIG. 21 is a diagram illustrating an updated image of a captured image generated in the fourth mode;

FIG. 22 is a diagram illustrating a configuration of the first and second imaging units in Modification Example 2 of the embodiment;

FIG. 23 is a diagram illustrating operations of the first imaging unit and the light source device in the third mode;

FIG. 24 is a diagram illustrating operations of the second imaging unit and the light source device in the third mode;

FIG. 25 is a diagram illustrating an updated image of a captured image generated in the third mode;

FIG. 26 is a diagram illustrating operations of the first imaging unit and the light source device in the fourth mode;

FIG. 27 is a diagram illustrating operations of the second imaging unit and the light source device in the fourth mode;

FIG. 28 is a diagram illustrating an updated image of a captured image generated in the fourth mode;

FIG. 29 is a diagram illustrating a configuration of the first and second imaging units in Modification Example 3 of the embodiment;

FIG. 30 is a diagram illustrating operations of the first imaging unit and the light source device in the third mode;

FIG. 31 is a diagram illustrating operations of the second imaging unit and the light source device in the third mode;

FIG. 32 is a diagram illustrating an updated image of a captured image generated in the third mode;

FIG. 33 is a diagram illustrating operations of the first imaging unit and the light source device in a fifth mode;

FIG. 34 is a diagram illustrating operations of the second imaging unit and the light source device in the fifth mode;

FIG. 35 is a diagram illustrating an updated image of a captured image generated in the fifth mode;

FIG. 36 is a diagram illustrating a configuration of the first and second imaging units in Modification Example 4 of the embodiment;

FIG. 37 is a diagram illustrating operations of the first imaging unit and the light source device in the third mode;

FIG. 38 is a diagram illustrating operations of the second imaging unit and the light source device in the third mode;

FIG. 39 is a diagram illustrating an updated image of a captured image generated in the third mode;

FIG. 40 is a diagram illustrating operations of the first imaging unit and the light source device in the second mode;

FIG. 41 is a diagram illustrating operations of the second imaging unit and the light source device in the second mode;

FIG. 42 is a diagram illustrating an updated image of a captured image generated in the second mode;

FIG. 43 is a diagram illustrating operations of the first imaging unit and the light source device in the first mode;

FIG. 44 is a diagram illustrating operations of the second imaging unit and the light source device in the first mode;

FIG. 45 is a diagram illustrating an updated image of a captured image generated in the first mode;

FIG. 46 is a diagram illustrating operations of the first imaging unit and the light source device in the fourth mode;

FIG. 47 is a diagram illustrating operations of the second imaging unit and the light source device in the fourth mode;

FIG. 48 is a diagram illustrating an updated image of a captured image generated in the fourth mode;

FIG. 49 is a diagram illustrating a configuration of the first and second imaging units in Modification Example 5 of the embodiment;

FIG. 50 is a diagram illustrating operations of the first imaging unit and the light source device in the third mode;

FIG. 51 is a diagram illustrating operations of the second imaging unit and the light source device in the third mode;

FIG. 52 is a diagram illustrating an updated image of a captured image generated in the third mode;

FIG. 53 is a diagram illustrating Modification Example 6 of the embodiment;

FIG. 54 is a diagram illustrating Modification Example 6 of the embodiment;

FIG. 55 is a diagram illustrating Modification Example 6 of the embodiment;

FIG. 56 is a diagram illustrating Modification Example 6 of the embodiment;

FIG. 57 is a diagram illustrating Modification Example 6 of the embodiment;

FIG. 58 is a diagram illustrating Modification Example 6 of the embodiment;

FIG. 59 is a diagram illustrating Modification Example 6 of the embodiment;

FIG. 60 is a diagram illustrating Modification Example 6 of the embodiment;

FIG. 61 is a diagram illustrating Modification Example 6 of the embodiment;

FIG. 62 is a diagram illustrating Modification Example 6 of the embodiment;

FIG. 63 is a diagram illustrating Modification Example 6 of the embodiment;

FIG. 64 is a diagram illustrating Modification Example 6 of the embodiment;

FIG. 65 is a diagram illustrating Modification Example 6 of the embodiment;

FIG. 66 is a diagram illustrating Modification Example 6 of the embodiment;

FIG. 67 is a diagram illustrating Modification Example 6 of the embodiment;

FIG. 68 is a diagram illustrating Modification Example 6 of the embodiment;

FIG. 69 is a diagram illustrating Modification Example 6 of the embodiment;

FIG. 70 is a diagram illustrating Modification Example 6 of the embodiment;

FIG. 71 is a diagram illustrating Modification Example 6 of the embodiment;

FIG. 72 is a diagram illustrating Modification Example 6 of the embodiment;

FIG. 73 is a diagram illustrating Modification Example 6 of the embodiment;

FIG. 74 is a diagram illustrating Modification Example 6 of the embodiment;

FIG. 75 is a diagram illustrating Modification Example 6 of the embodiment;

FIG. 76 is a diagram illustrating Modification Example 6 of the embodiment;

FIG. 77 is a diagram illustrating Modification Example 6 of the embodiment;

FIG. 78 is a diagram illustrating Modification Example 6 of the embodiment;

FIG. 79 is a diagram illustrating Modification Example 6 of the embodiment;

FIG. 80 is a diagram illustrating Modification Example 6 of the embodiment;

FIG. 81 is a diagram illustrating Modification Example 6 of the embodiment;

FIG. 82 is a diagram illustrating Modification Example 7 of the embodiment;

FIG. 83 is a diagram illustrating Modification Example 8 of the embodiment;

FIG. 84 is a diagram illustrating Modification Example 9 of the embodiment; and

FIG. 85 is a diagram illustrating Modification Example 9 of the embodiment;

### DETAILED DESCRIPTION

Hereinafter, an embodiment of the present disclosure (hereinafter, the embodiment) will be described with reference to the drawings. The present disclosure is not limited to the embodiment described below. Further, in the drawings, the same portions are represented by the same reference numerals.

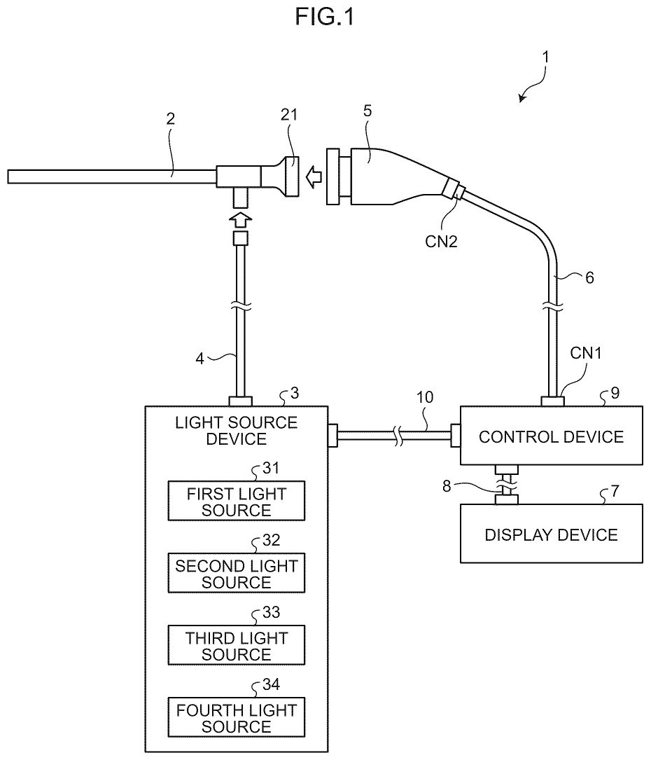

FIG. 1 is a diagram illustrating a configuration of a medical stereoscopic observation system 1 according to the embodiment.

In the present embodiment, the medical stereoscopic observation system 1 is a medical endoscope system that stereoscopically observes an observation target (the inside of a living body) using an endoscope. As illustrated in FIG. 1, the medical stereoscopic observation system 1 includes an insertion unit 2, a light source device 3, a light guide 4, a camera head 5, a first transmission cable 6, a display device 7, a second transmission cable 8, a control device 9, and a third transmission cable 10.

In the present embodiment, the insertion unit 2 is configured with a rigid endoscope. That is, the insertion unit 2 has an elongated shape where the entire portion is hard or a part is soft and the other portion is hard, and is inserted into the observation target. In the insertion unit 2, an optical system that is configured with one or a plurality of lenses and focuses return light (subject image) from the observation target is provided. As the insertion unit 2, not only a general scope (scope where one optical path is set in the scope) but also a binocular relay type or a monocular pupil-division type scope may be adopted.

In the binocular relay type scope, two optical paths are arranged in parallel in the scope. In addition, an optical system is disposed in each of the two optical paths. In the binocular relay type scope, observation light components for right and left eyes are having parallax with each other are taken into the two optical systems and emitted therefrom (for example, refer to JP H6-160731 A).

In addition, in the monocular pupil-division type scope, one optical path is provided in the scope. In addition, an optical system is disposed in the one optical path. Further, at a pupil position of the optical system, a pupil division unit that divides a luminous flux in the pupil into two parts for two regions is provided. In the monocular pupil-division type scope, observation light is taken into the optical system, the observation light is separated into observation light components for right and left eyes having parallax with each other by the pupil division unit, and the separated light components are emitted (for example, JP H6-59199 A).

One end of the light guide 4 is connected to the light source device 3. The light source device 3 includes a first light source 31 (FIG. 1) that supplies normal light including at least a part of a wavelength band of visible light (hereinafter, referred to as white light) to the one end of the light guide 4 under the control of the control device 9, a second light source 32 (FIG. 1) that supplies first excitation light as narrow-band light to the one end of the light guide 4, a third light source 33 (FIG. 1) that supplies second excitation light as narrow-band light to the one end of the light guide 4, and a fourth light source 34 (FIG. 1) that supplies third excitation light as narrow-band light to the one end of the light guide 4. The first to third excitation light components may be visible light or invisible light. In addition, the first to fourth light sources 31 to 34 may be configured with light emitting diodes (LEDs) or semiconductor lasers. In addition, the number of the first light sources 31 that emit the white light may be one or plural. The number of the second light sources 32 that emit the first excitation light may also be one or plural. The number of the third light sources 33 that emit the second excitation light may also be one or plural. The number of the fourth light sources 34 that emit the third excitation light may also be one or plural.

Here, examples of a substance in the observation target that is excited by the first to third excitation light components include a chemical agent or a fluorescent dye that is added to the observation target and a fluorescent substance derived from the observation target forming the observation target itself.

Examples of the above-described chemical agent added to the observation target include “5-ALA(PP-IX)”, “ADS780WS”, “ADS830WS”, “aggregation-induced emission dots allophycocyanin (APC)”, “boron-dipyrromethane (BODIPY)”, “CLR 1502”, “Flavins”, “fluorescamine”, “Fluorescein”, “fluoro-gold”, “green fluorescence protein”, “ICG (indocyanine green)”, “IRDye 78”, “IR-PEG nanoparticles”, “Isothiocyanate”, “rose Bengal”, “SGM-101”, and “trypan blue”.

In addition, examples of the above-described fluorescent dye added to the observation target OB include “coumarine”, “Cy3”, “DyLight547”, “GE3126”, “metal nanoclusters”, “oxacarbocyanine”, “Rhodamine”, “Riboflavin”, “fluorescein”, “AlexaFluor 488”, “AlexaFluor660”, “AlexaFluor680”, “AlexaFluor700”, “Cy5”, “Cy5.5”, “Dy677”, “Dy682”, “Dy752”, “DyLight647”, “HiLyte Fluor 647”, “HiLyte Fluor 680”, “IRDye 700DX”, “methylene blue”, “Porphyrins”, “Porphysomes”, “VivoTag-680”, “VivoTag-S680”, “AlexaFluor750”, “AlexaFluor790”, “carbocyanine”, “conjugated copolymers”, “CW800-CA”, “Cy7”, “Cy7.5”, “cyanine dyes”, “Dy780”, “HiLyte Fluor 750”, “Indocarbocyanine”, “IR-786”, “IRDye 800CW”, “IRDye 800RS”, “IRDye 800BK”, “Nervelight”, “OTL-38(Pafolacianine)”, “Polymethine”, “VivoTag-S750”, “ASP5354”, “Xanthene”, and “LUM-015”.

Further examples of the fluorescent substance derived from the observation target forming the observation target itself include “collagen”, “elastin”, and “NADH”.

In the present embodiment, the light source device 3 is configured separately from the control device 9, but the present embodiment is not limited thereto. A configuration where the light source device 3 and the control device 9 are provided in the same casing may also be adopted.

The one end of the light guide 4 is detachably connected to the light source device 3. In addition, the other end of the light guide 4 is detachably connected to the insertion unit 2. The light guide 4 allows the white light and the first to third excitation light components supplied from the light source device 3 (first to fourth light sources 31 to 34) to propagate from the one end to the other end and supply the white light and the first to third excitation light components to the insertion unit 2, respectively. The white light and the first to third excitation light components supplied to the insertion unit 2 are emitted from a distal end of the insertion unit 2 such that the observation target is irradiated with the emitted light. Each of the components of the return light (subject image) of the white light and the first to third excitation light components that are reflected from the observation target by irradiation of the observation target is focused in the optical system in the insertion unit 2. The return light of the white light is the white light reflected from the observation target. The return light of the first excitation light includes not only the first excitation light reflected from the observation target but also fluorescence (hereinafter, referred to as first fluorescence) that is emitted from a substance in the observation target when the observation target is irradiated with the first excitation light such that the substance is excited. The return light of the second excitation light includes not only the second excitation light reflected from the observation target but also fluorescence (hereinafter, referred to as second fluorescence) that is emitted from a substance in the observation target when the observation target is irradiated with the second excitation light such that the substance is excited. The return light of the third excitation light includes not only the third excitation light reflected from the observation target but also fluorescence (hereinafter, referred to as third fluorescence) that is emitted from a substance in the observation target when the observation target is irradiated with the third excitation light such that the substance is excited.

Here, the first fluorescence and the second and third fluorescence components have different wavelength bands. In addition, the second and third fluorescence components have a similar wavelength band. The third fluorescence has a stronger fluorescence intensity than the second fluorescence.

The camera head 5 corresponds to the medical stereoscopic observation imaging device according to the present disclosure. This camera head 5 is detachably connected to a proximal end (eyepiece unit 21 (FIG. 1)) of the insertion unit 2. The camera head 5 separates the light focused in the insertion unit 2 into observation light for right eye and observation light for left eye. Here, the observation light for right eye includes normal observation light for right eye (first normal observation light according to the present disclosure) separated from the return light of the white light and fluorescence observation light for right eye separated from the first to third fluorescence components. Here, the observation light for left eye includes normal observation light for left eye (second normal observation light according to the present disclosure) separated from the return light of the white light and fluorescence observation light for left eye separated from the first to third fluorescence components. In addition, the camera head 5 captures each of the normal observation light for right eye, the normal observation light for left eye, and the first to third fluorescence components to generate an image signal under the control of the control device 9. Hereinafter, for convenience of the description, the image signal obtained by capturing the normal observation light for right eye will be referred to as a normal observation image for right eye (corresponding to the first normal observation image according to the present disclosure). In addition, the image signal obtained by capturing the normal observation light for left eye will be referred to as a normal observation image for left eye (corresponding to the second normal observation image according to the present disclosure). Further, the image signal obtained by capturing the first fluorescence will be referred to as a first fluorescence observation image. In addition, the image signal obtained by capturing the second fluorescence will be referred to as a second fluorescence observation image. Further, the image signal obtained by capturing the third fluorescence will be referred to as a third fluorescence observation image. In addition, the normal observation image for right eye, the normal observation image for left eye, and the first to third fluorescence observation images will be collectively referred to as the captured image.

The detailed configuration of the camera head 5 will be described in “Configuration of Camera Head” described below.

One end CN1 of the first transmission cable 6 is detachably connected to the control device 9. In addition, the other end CN2 of the first transmission cable 6 is detachably connected to the camera head 5. The other end CN2 is not limited to the configuration that is detachably connected to the camera head 5, and a configuration where the other end CN2 is fixed to the camera head 5 may also be adopted. The first transmission cable 6 transmits the captured image output from the camera head 5 to the control device 9, and transmits each of a control signal, a synchronization signal, a clock, power, and the like transmitted from the control device 9 to the camera head 5.

The captured image and the like transmitted from the camera head 5 to the control device 9 through the first transmission cable 6 may be transmitted as an optical signal or as an electrical signal. The same also applies to the transmission of the control signal, the synchronization signal, and the clock from the control device 9 to the camera head 5 through the first transmission cable 6.

The display device 7 is configured with a display using a liquid crystal, an organic electro luminescence (EL), or the like, and displays an image based on a video signal from the control device 9 under the control of the control device 9.

One end of the second transmission cable 8 is detachably connected to the display device 7. In addition, the other end of the second transmission cable 8 is detachably connected to the control device 9. The second transmission cable 8 transmits the video signal processed by the control device 9 to the display device 7.

The control device 9 includes a controller such as a central processing unit (CPU) or a micro processing unit (MPU), and integrally controls operations of the light source device 3, the camera head 5, and the display device 7. The control device 9 is not limited to a CPU or an MPU, and may include an application specific integrated circuit (ASIC), a field-programmable gate array (FPGA), a graphics processing unit (GPU), or the like.

The detailed configuration of the control device 9 will be described in “Configuration of Control Device” described below.

One end of the third transmission cable 10 is detachably connected to the light source device 3. In addition, the other end of the third transmission cable 10 is detachably connected to the control device 9. The third transmission cable 10 transmits the control signal from the control device 9 to the light source device 3.

Next, the configuration of the camera head 5 will be described.

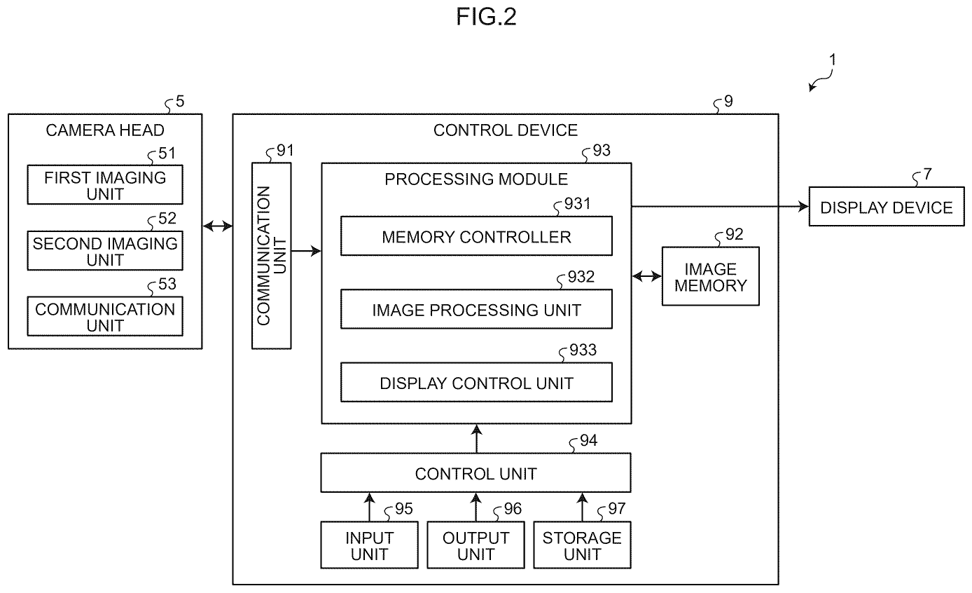

FIG. 2 is a block diagram illustrating a configuration of the camera head 5 and the control device 9.

As illustrated in FIG. 2, the camera head 5 includes first and second imaging units 51 and 52 and a communication unit 53.

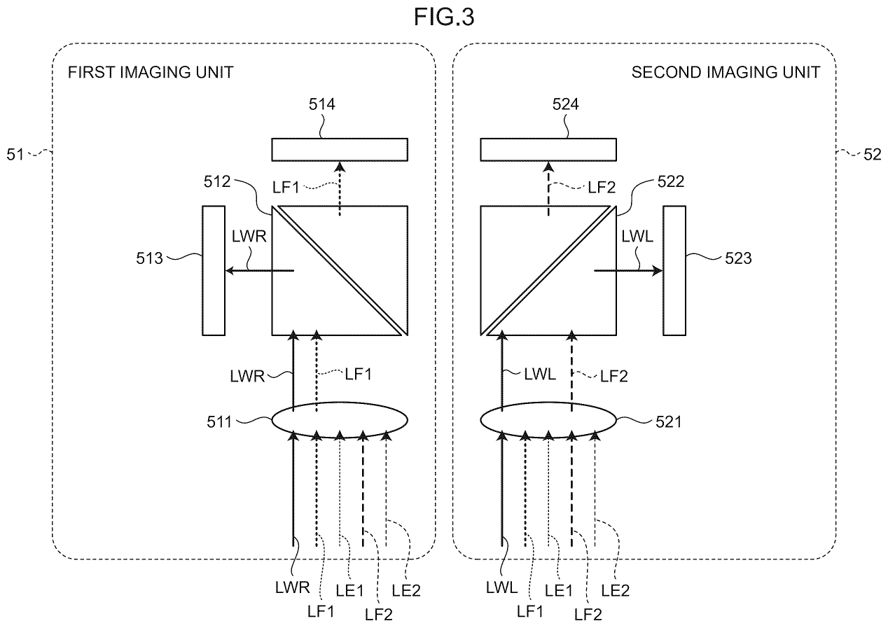

FIG. 3 is a diagram illustrating a configuration of the first and second imaging units 51 and 52. In FIG. 2, the normal observation light for right eye and the normal observation light for left eye having parallax with each other that are separated from the return light of the white light in the camera head 5 are represented by normal observation light for right eye LWR and normal observation light for left eye LWL. In addition, in FIG. 2, the first excitation light is represented by excitation light LE1, and the first fluorescence is represented by fluorescence LF1. In FIG. 2, the second and third excitation light components are collectively represented by excitation light LE2, and the second and third fluorescence components are represented by fluorescence LF2.

The first imaging unit 51 captures the normal observation light for right eye LWR and the fluorescence LF1 to generate the normal observation image for right eye and the first fluorescence observation image, respectively. As illustrated in FIG. 3, the first imaging unit 51 includes first and second optical members 511 and 512 and first and second image sensors 513 and 514.

In the present embodiment, the first optical member 511 is configured with a filter that cuts light in a specific wavelength band. Here, “cut” represents that light is partially, substantially, or completely suppressed. “Cut” described below has the same meaning. The first optical member 511 is not limited to the filter, and may also be configured with another optical member as long as it has a function of separating light in a specific wavelength band from light in another wavelength band.

Specifically, as illustrated in FIG. 3, the first optical member 511 has a light cutting function of cutting the excitation light components LE1 and LE2 and the fluorescence LF2 in the light that is focused in the insertion unit 2 and is incident on the first optical member 511. The light that is focused in the insertion unit 2 and is incident on the first optical member 511 includes the normal observation light for right eye LWR, the excitation light components LE1 and LE2, and the fluorescence components LF1 and LF2.

In the present embodiment, the second optical member 512 is configured with a prism that separates light in a specific wavelength band from light in another wavelength band. The second optical member 512 is not limited to the prism, and may also be configured with another optical member as long as it has a function of separating light in a specific wavelength band from light in another wavelength band.

Specifically, as illustrated in FIG. 3, the second optical member 512 has a light separating function of separating the normal observation light for right eye LWR and the fluorescence LF1 that are not cut by the first optical member 511. The second optical member 512 allows the normal observation light for right eye LWR to travel toward the first image sensor 513, and allows the fluorescence LF1 to travel toward the second image sensor 514.

The first image sensor 513 is an image sensor such as a charge coupled device (CCD) or a complementary metal oxide semiconductor (CMOS) that receives and converts light into an electrical signal. The first image sensor 513 captures the normal observation light for right eye LWR through the second optical member 512 under the control of the control device 9. The normal observation image for right eye is obtained by the capturing.

The second image sensor 514 is an image sensor such as a CCD or a CMOS. The second image sensor 514 captures the fluorescence LF1 through the second optical member 512 under the control of the control device 9. The first fluorescence observation image is obtained by the capturing.

The second imaging unit 52 captures the normal observation light for left eye LWL and the fluorescence LF2 to generate the normal observation image for left eye and the second fluorescence observation image or third fluorescence observation image, respectively. As illustrated in FIG. 3, the second imaging unit 52 includes third and fourth optical members 521 and 522 and third and fourth image sensors 523 and 524.

In the present embodiment, the third optical member 521 is configured with a filter that cuts light in a specific wavelength band. The third optical member 521 is not limited to the filter, and may also be configured with another optical member as long as it has a function of separating light in a specific wavelength band from light in another wavelength band.

Specifically, as illustrated in FIG. 3, the third optical member 521 has a light cutting function of cutting the excitation light components LE1 and LE2 and the fluorescence LF1 in the light that is focused in the insertion unit 2 and is incident on the third optical member 521. The light that is focused in the insertion unit 2 and is incident on the third optical member 521 includes the normal observation light for left eye LWL, the excitation light components LE1 and LE2, and the fluorescence components LF1 and LF2.

In the present embodiment, the fourth optical member 522 is configured with a prism that separates light in a specific wavelength band from light in another wavelength band. The fourth optical member 522 is not limited to the prism, and may also be configured with another optical member as long as it has a function of separating light in a specific wavelength band from light in another wavelength band.

Specifically, as illustrated in FIG. 3, the fourth optical member 522 has a light separating function of separating the normal observation light for left eye LWL and the fluorescence LF2 that are not cut by the third optical member 521. The fourth optical member 522 allows the normal observation light for left eye LWL to travel toward the third image sensor 523, and allows the fluorescence LF2 to travel toward the fourth image sensor 524.

The third image sensor 523 is an image sensor such as a CCD or a CMOS. The third image sensor 523 captures the normal observation light for left eye LWL through the fourth optical member 522 under the control of the control device 9. The normal observation image for left eye is obtained by the capturing.

The fourth image sensor 524 is an image sensor such as a CCD or a CMOS. The fourth image sensor 524 captures the fluorescence LF2 (the second fluorescence or the third fluorescence) through the fourth optical member 522 under the control of the control device 9. The second fluorescence observation image or the third fluorescence observation image is obtained by the capturing.

Here, the number of pixels in the normal observation image for right eye, the number of pixels in the normal observation image for left eye, the number of pixels in the first fluorescence observation image, and the number of pixels in the second fluorescence observation image may be all different, or at least two of the images may have the same number of pixels.

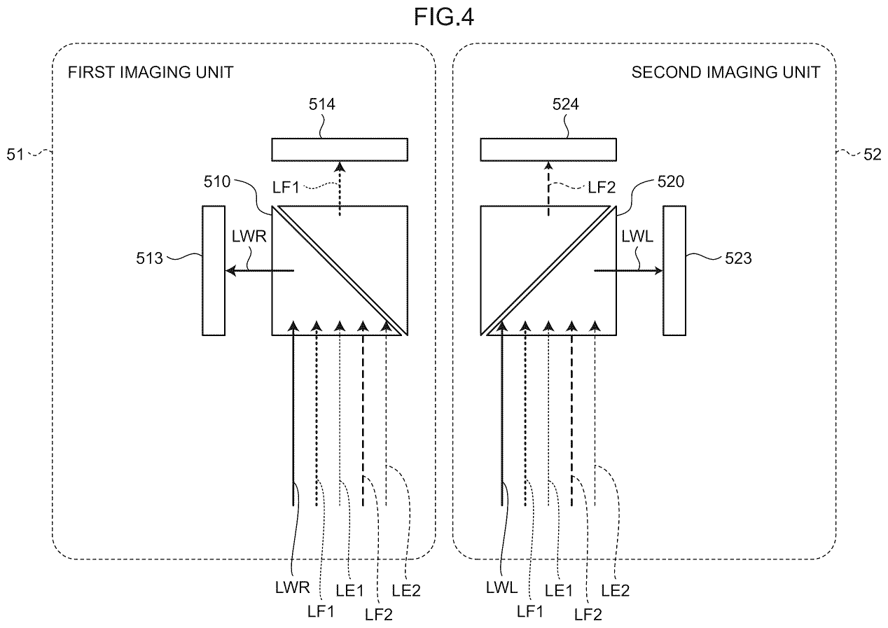

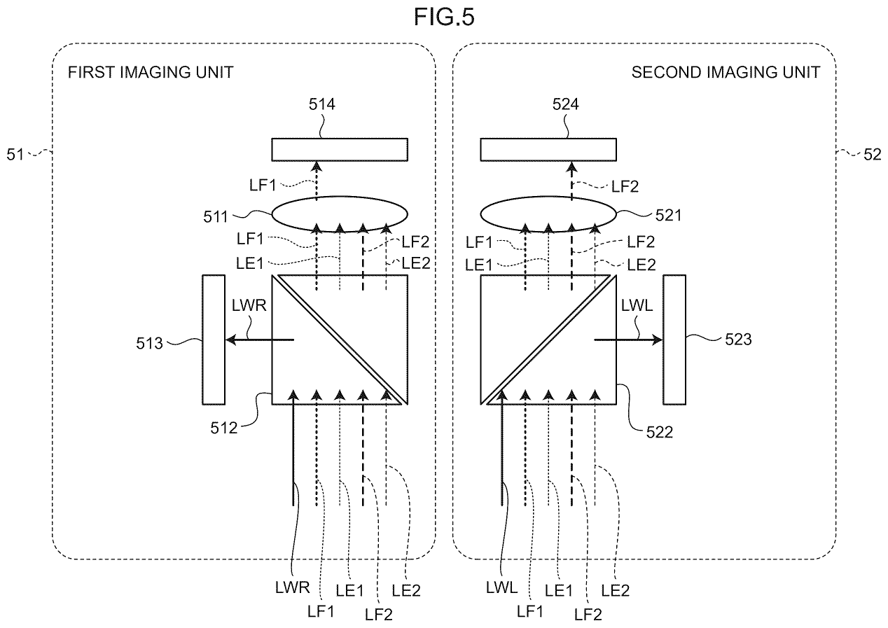

FIGS. 4 and 5 are diagrams illustrating other examples of a configuration of the first and second imaging units 51 and 52.

The configuration of the first and second imaging units 51 and 52 described above is not limited to the configuration illustrated in FIG. 3 and may be the configuration illustrated in FIG. 4 or 5.

In the first imaging unit 51 illustrated in FIG. 4, one optical member 510 has the above-described light cutting function in the first optical member 511 and the above-described light separating function in the second optical member 512.

Likewise, in the second imaging unit 52 illustrated in FIG. 4, one optical member 520 has the above-described light cutting function in the third optical member 521 and the above-described light separating function in the fourth optical member 522.

In the first imaging unit 51 illustrated in FIG. 5, the first optical member 511 is disposed between the second optical member 512 and the second image sensor 514.

The light separating function in the second optical member 512 is the function of separating the light that is focused in the insertion unit 2 and is incident on the second optical member 512 into the normal observation light for right eye LWR, the excitation light components LE1 and LE2, and the fluorescence components LF1 and LF2. In addition, the second optical member 512 allows the normal observation light for right eye LWR to travel toward the first image sensor 513. Further, the second optical member 512 allows the excitation light components LE1 and LE2, the fluorescence LF1, and the fluorescence LF2 to travel toward the first optical member 511.

In addition, the light cutting function in the first optical member 511 is the function of cutting the excitation light components LE1 and LE2 and the fluorescence LF2 in the light that is incident on the first optical member 511. The fluorescence LF1 that is not cut by the first optical member 511 travels toward the second image sensor 514.

Likewise, in the second imaging unit 52 illustrated in FIG. 5, the third optical member 521 is disposed between the fourth optical member 522 and the fourth image sensor 524.

The light separating function in the fourth optical member 522 is the function of separating the light that is focused in the insertion unit 2 and is incident on the fourth optical member 522 into the normal observation light for left eye LWL, the excitation light components LE1 and LE2, and the fluorescence components LF1 and LF2. In addition, the fourth optical member 522 allows the normal observation light for left eye LWL to travel toward the third image sensor 523. Further, the fourth optical member 522 allows the excitation light components LE1 and LE2, the fluorescence components LF1 and LF2 to travel toward the third optical member 521.

In addition, the light cutting function in the third optical member 521 is the function of cutting the excitation light components LE1 and LE2 and the fluorescence LF1 in the light that is incident on the third optical member 521. The fluorescence LF2 that is not cut by the third optical member 521 travels toward the fourth image sensor 524.

Table 1 shown below is a table showing a correspondence between the first to fourth image sensors 513, 514, 523, and 524, the white light, and the first to third fluorescence components.

TABLE 1

| | First | Second | Third | Fourth |
|---|---|---|---|---|
| | image | image | image | image |
| | sensor | sensor | sensor | sensor |
| White light | ⊚ | X | ⊚ | X |
| First | X | ◯ | X | X |
| fluorescence | | | | |
| Second | X | X | X | ◯ |
| fluorescence | | | | |
| Third | X | X | X | ◯ |
| fluorescence | | | | |

In Table 1, “⊚” represents being capturable three-dimensionally (in 3D). That is, a 3D image can be generated by the normal observation image for right eye generated in the first image sensor 513 and the normal observation image for left eye generated in the third image sensor 523. In addition, “◯” represents being capturable two-dimensionally (in 2D). That is, the 2D first fluorescence observation image can be generated by the second image sensor 514. In addition, each of the 2D second and third fluorescence observation images can be generated by the fourth image sensor 524. Further, “X” represents not being supported.

The communication unit 53 executes signal processing on the captured image (analog signal) generated by the first to fourth image sensors 513, 514, 523, and 524, and outputs captured image (digital signal) under the control of the control device 9.

Examples of the signal processing that is executed by the communication unit 53 include the following signal processing.

For example, the communication unit 53 executes signal processing such as processing of removing reset noise, processing of multiplying an analog gain for amplifying an analog signal, and A/D conversion on the captured image (analog signal) generated by the first to fourth image sensors 513, 514, 523, and 524.

The communication unit 53 functions as a transmitter that transmits the captured image on which the above-described signal processing is executed to the control device 9 through the first transmission cable 6. The communication unit 53 is configured with, for example, a high-speed serial interface that executes communication of the captured image with the control device 9 through the first transmission cable 6 at a transmission rate of 1 Gbps or higher.

Next, the configuration of the control device 9 will be described with reference to FIG. 2.

As illustrated in FIG. 2, the control device 9 includes a communication unit 91, an image memory 92, a processing module 93, a control unit 94, an input unit 95, an output unit 96, and a storage unit 97.

The communication unit 91 functions as a receiver that receives the captured image sequentially transmitted from the camera head 5 (communication unit 53) through the first transmission cable 6. The communication unit 91 is configured with, for example, a high-speed serial interface that executes communication of the captured image with the communication unit 53 at a transmission rate of 1 Gbps or higher.

The image memory 92 is configured with, for example, a dynamic random access memory (DRAM) or the like. The image memory 92 can temporarily store the captured image corresponding to a plurality of frames sequentially output from the camera head 5 (communication unit 53).

The processing module 93 corresponds to the medical image processing device according to the present disclosure. The processing module 93 processes the captured image that is sequentially transmitted from the camera head 5 (communication unit 53) and received by the communication unit 91 under the control of the control unit 94. As illustrated in FIG. 2, the processing module 93 includes a memory controller 931, an image processing unit 932, and a display control unit 933.

The memory controller 931 controls writing of the captured image into the image memory 92 and reading of the captured image from the image memory 92. The captured image read by the memory controller 931 is input to the image processing unit 932.

The image processing unit 932 executes image processing on the input captured image.

Examples of the image processing include optical black subtraction processing (clamp processing), white balance adjustment processing, demosaic processing, color correction matrix processing, gamma correction processing, YC processing of converting an RGB signal into a luminance-color difference signal (Y, Cb/Cr signal), digital gain adjustment of multiplying a digital gain, noise removal, and filter process of executing structure emphasis.

The image processing that is executed on the normal observation image for right eye, the image processing that is executed on the normal observation image for left eye, the image processing that is executed on the first fluorescence observation image, the image processing that is executed on the second fluorescence observation image, and the image processing that is executed on the third fluorescence observation image may be all different, or the same image processing may be executed on at least two of the images.

The display control unit 933 generates a video signal for displaying the captured image on which the image processing is executed by the image processing unit 932 under the control of the control unit 94. The display control unit 933 outputs the video signal to the display device 7 through the second transmission cable 8.

The control unit 94 is implemented by a controller such as a CPU or an MPU executing various programs stored in the storage unit 97, controls the operations of the light source device 3, the camera head 5, and the display device 7, and controls the entire operation of the control device 9. The control unit 94 is not limited to the CPU or the MPU, and may include an ASIC, a FPGA, or a GPU. The function of the control unit 94 will be described in “Operation of Medical stereoscopic observation system” described below.

The input unit 95 is configured with an operation device such as a mouse, a keyboard, or a touch panel, and receives a user operation from a user such as an operator. The input unit 95 outputs an operation signal corresponding to the user operation to the control unit 94.

The output unit 96 is configured using a speaker, a printer, or the like, and outputs various information.

The storage unit 97 stores the programs that are executed by the control unit 94, information required for the processing of the control unit 94, and the like.

### Operation of Medical Stereoscopic Observation System

Next, the operation of the above-described medical stereoscopic observation system 1 will be described.

The medical stereoscopic observation system 1 is set to, for example, each of first to fourth modes according to the user operation from the user to the input unit 95. The medical stereoscopic observation system 1 executes different operations depending on the first to fourth modes.

Hereinafter, the operations corresponding to the first to fourth modes will be sequentially described.

The first mode is a mode of generating the normal observation image for right eye, the normal observation image for left eye, and the first fluorescence observation image.

Hereinafter, operations of the first imaging unit 51 and the light source device 3 in the first mode, operations of the second imaging unit 52 and the light source device 3 in the first mode, and an updated image of the captured image generated in the first mode will be sequentially described.

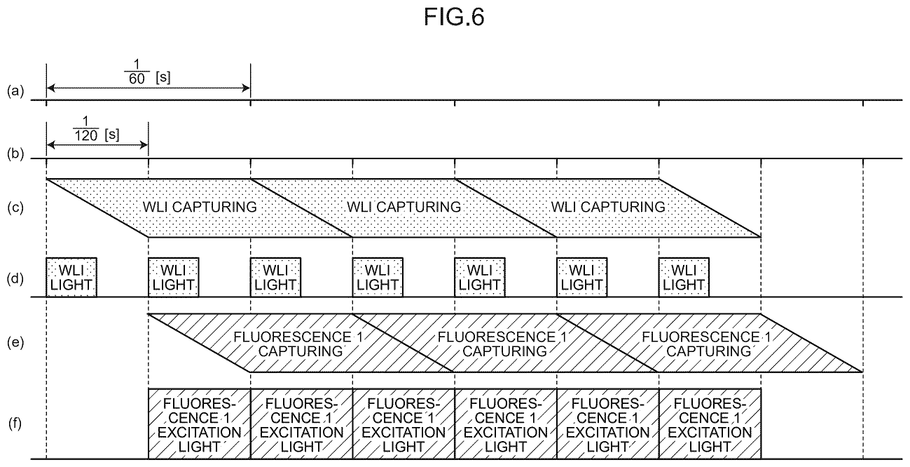

FIG. 6 is a diagram illustrating the operations of the first imaging unit 51 and the light source device 3 in the first mode. Specifically, (a) of FIG. 6 represents a first synchronization signal. Hereinafter, for convenience of description, a configuration adopting an NTSC system is assumed. The configuration is not limited to the NTSC system, and a configuration adopting another system such as a PAL system may be assumed. Since the NTSC system is adopted, the first synchronization signal is a signal with a period of 1/60 [s](a period of a frame (field)). (b) of FIG. 6 represents a second synchronization signal with a period that is ½ of the period of the first synchronization signal. (c) of FIG. 6 is a diagram illustrating a capturing control of the first image sensor 513, in which the vertical axis represents a horizontal line of the first image sensor 513 (the uppermost stage represents the uppermost horizontal line (first horizontal line), and the lowermost stage represents the lowermost horizontal line (final line)), and the horizontal axis represents the time. A parallelogram region is a region contributing to the generation of the normal observation image for right eye in one frame (field). For convenience of description, in (c) of FIG. 6, a text “WLI Capturing” representing the capturing of the normal observation light for right eye LWR (white light) is illustrated in the parallelogram regions. (d) of FIG. 6 is a diagram illustrating a light source control of the first light source 31, in which the vertical axis represents a power value [W] that is supplied to the first light source 31, and the horizontal axis represents the time (the supply time of the power supplied to the first light source 31). In the present embodiment, a voltage value that is supplied to the first light source 31 is fixed. Therefore, in (d) of FIG. 6, the vertical axis corresponds to a current value that is supplied to the first light source 31. (e) of FIG. 6 is a diagram illustrating a capturing control of the second image sensor 514, in which the vertical axis represents a horizontal line of the second image sensor 514 (the uppermost stage represents the uppermost horizontal line (first horizontal line), and the lowermost stage represents the lowermost horizontal line (final line)), and the horizontal axis represents the time. A parallelogram region is a region contributing to the generation of the first fluorescence observation image in one frame (field). For convenience of description, in (e) of FIG. 6, a text “Fluorescence 1 Capturing” representing the capturing of the fluorescence LF1 (first fluorescence) is illustrated in the parallelogram regions. (f) of FIG. 6 is a diagram illustrating a light source control of the second light source 32, in which the vertical axis represents a power value [W] that is supplied to the second light source 32, and the horizontal axis represents the time (the supply time of the power supplied to the second light source 32). In the present embodiment, a voltage value that is supplied to the second light source 32 is fixed. Therefore, in (f) of FIG. 6, the vertical axis corresponds to a current value that is supplied to the second light source 32.

When set to the first mode, the control unit 94 controls the operations of the first imaging unit 51 and the light source device 3 as described below.

As illustrated in (c) of FIG. 6, the control unit 94 executes a capturing control using a so-called rolling shutter system of sequentially starting exposure in one field period of the first image sensor 513 for each horizontal line and sequentially executing reading for each horizontal line where a predetermined period (so-called shutter speed) is elapsed from the exposure start. In the example of (c) of FIG. 6, in the first image sensor 513, the all-line exposure period is set to 1/120 [s], and reading is executed at 1/120 [s]. The all-line exposure period or the reading speed may be set to another value.

In addition, as illustrated in (d) of FIG. 6, the control unit 94 supplies power to the first light source 31 in a period of 1/120 [s] at a timing of the second synchronization signal. Here, the supply time of the power supplied to the first light source 31 is shorter than 1/120 [s]. As a result, the first light source 31 emits the white light in a period of 1/120 [s] for a time of shorter than 1/120 [s]. In (d) of FIG. 6, for convenience of description, a text “WLI light” representing emission of the white light is illustrated in rectangular regions representing the supply state of power.

Further, as illustrated in (e) of FIG. 6, as in the capturing control of the first image sensor 513, the control unit 94 executes a capturing control using a rolling shutter system for the second image sensor 514. At this time, read timings in the respective fields of the first and second image sensors 513 and 514 are shifted from each other by 1/120 [s] as illustrated in (c) and (e) of FIG. 6. The read timings of the respective fields may be shifted from each other or may be the same.

In addition, as illustrated in (f) of FIG. 6, the control unit 94 constantly supplies power to the second light source 32. As a result, the second light source 32 is constantly turned on to constantly emit the first excitation light. In (f) of FIG. 6, for convenience of description, a text “Fluorescence 1 Excitation Light” representing emission of the first excitation light is illustrated in rectangular regions representing the supply state of power.

Through the operation of the first imaging unit 51 and the operation of the light source device 3 described above, the normal observation image for right eye and the first fluorescence observation image (fluorescence observation light for right eye) are generated.

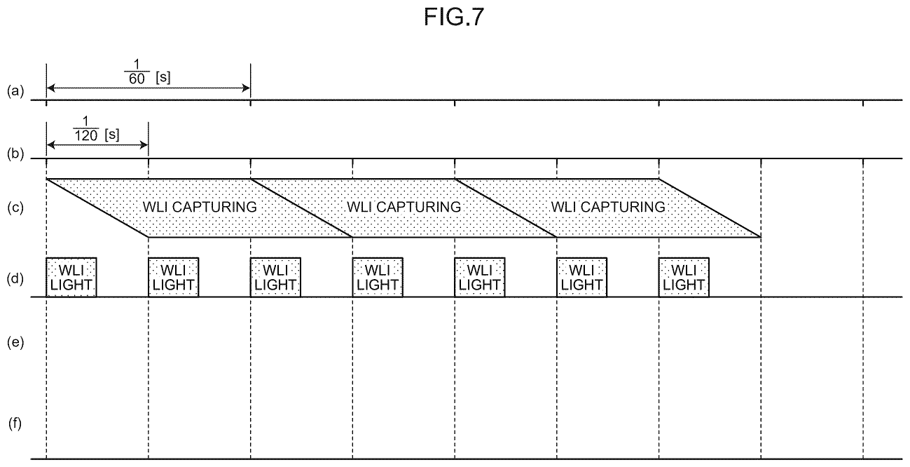

FIG. 7 is a diagram illustrating the operations of the second imaging unit 52 and the light source device 3 in the first mode. Specifically, (a) and (b) of FIG. 7 are diagrams corresponding to (a) and (b) of FIG. 6, respectively. (c) of FIG. 7 is a diagram illustrating a capturing control of the third image sensor 523, in which the vertical axis represents a horizontal line of the third image sensor 523 (the uppermost stage represents the uppermost horizontal line (first horizontal line), and the lowermost stage represents the lowermost horizontal line (final line)), and the horizontal axis represents the time. A parallelogram region is a region contributing to the generation of the normal observation image for left eye in one frame (field). For convenience of description, in (c) of FIG. 7, a text “WLI Capturing” representing the capturing of the normal observation light for left eye LWL (white light) is illustrated in the parallelogram regions. (d) of FIG. 7 is a diagram corresponding to (d) of FIG. 6. (e) in FIG. 7 is a diagram illustrating a capturing control of the fourth image sensor 524. (f) in FIG. 7 is a diagram illustrating a light source control of the third and fourth light sources 33 and 34.

When set to the first mode, the control unit 94 controls the operations of the second imaging unit 52 and the light source device 3 as described below.

As illustrated in (c) of FIG. 7, as in the capturing control of the first image sensor 513, the control unit 94 executes a capturing control using a rolling shutter system for the third image sensor 523. At this time, read timings in the respective fields of the first and third image sensors 513 and 523 are the same as illustrated in (c) of FIG. 6 and (c) of FIG. 7.

Through the operation of the second imaging unit 52 and the operation of the light source device 3 described above, the normal observation image for left eye is generated.

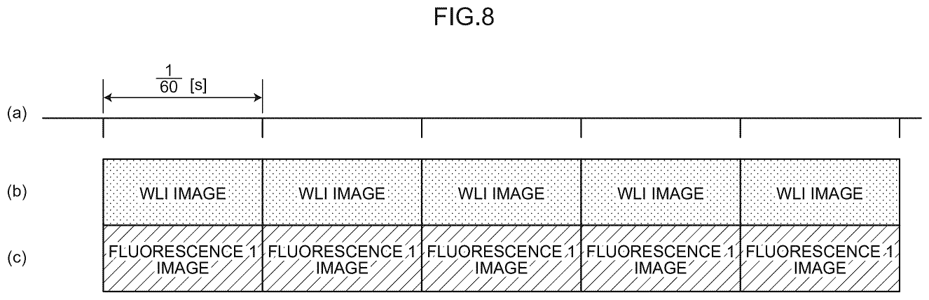

FIG. 8 is a diagram illustrating the updated image of the captured image generated in the first mode. Specifically, (a) of FIG. 8 is a diagram corresponding to (a) of FIG. 6. (b) of FIG. 8 is a diagram illustrating updated images of the normal observation image for right eye and the normal observation image for left eye. In (b) of FIG. 8, for convenience of description, a text “WLI image” representing the normal observation image for right eye and the normal observation image for left eye is illustrated in rectangular regions representing the normal observation image for right eye and the normal observation image for left eye. (c) of FIG. 8 is a diagram illustrating the updated image of the first fluorescence observation image. For convenience of description, in (c) of FIG. 8, a text “Fluorescence 1 Image” representing the first fluorescence observation image is illustrated in rectangular regions representing the first fluorescence observation image.

Through the operations of the first and second imaging units 51 and 52 and the operation of the light source device 3 described above, each of the normal observation image for right eye, the normal observation image for left eye, and the first fluorescence observation image (fluorescence observation light for right eye) that are generated is updated for each frame (period of 1/60 [s]) as illustrated in FIG. 8.

The second mode is a mode of generating the normal observation image for right eye, the normal observation image for left eye, and the second fluorescence observation image.

The third mode is a mode of generating the normal observation image for right eye, the normal observation image for left eye, and the third fluorescence observation image.

In the present embodiment, the first and second imaging units 51 and 52 and the light source device 3 operate in the same manner as in the second and third modes. Therefore, hereinafter, the operations corresponding to the second and third modes will be collectively described.

Hereinafter, operations of the first imaging unit 51 and the light source device 3 in the second and third modes, operations of the second imaging unit 52 and the light source device 3 in the second and third modes, and an updated images of the captured image generated in the second and third modes will be sequentially described.

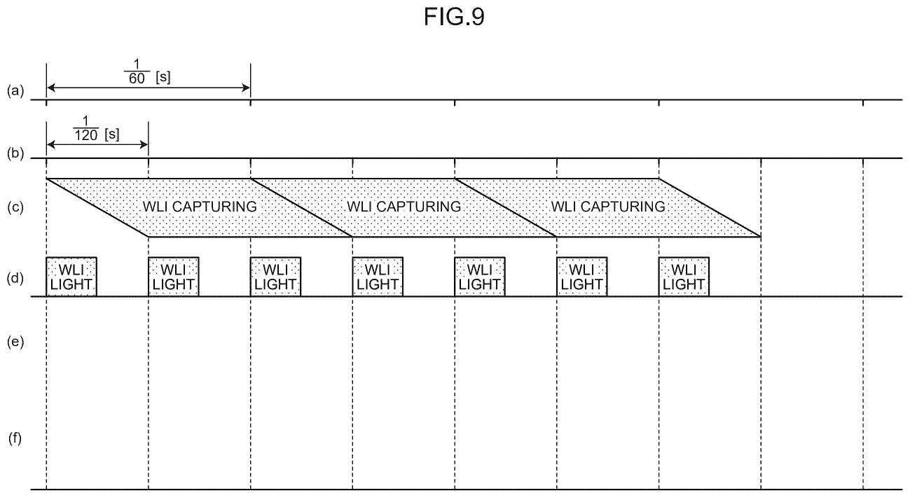

FIG. 9 is a diagram illustrating the operations of the first imaging unit 51 and the light source device 3 in the second and third modes. Specifically, (a) to (f) of FIG. 9 are diagrams corresponding to (a) to (f) of FIG. 6, respectively.

When set to the second and third modes, the control unit 94 controls the operations of the first imaging unit 51 and the light source device 3 as described below.

As illustrated in (c) of FIG. 9, the control unit 94 executes a capturing control of the first image sensor 513 as in the case where the control unit 94 is set to the first mode. In addition, as illustrated in (d) of FIG. 9, the control unit 94 executes a light source control of the first light source 31 as in the case where the control unit 94 is set to the first mode.

Through the operation of the first imaging unit 51 and the operation of the light source device 3 described above, the normal observation image for right eye is generated.

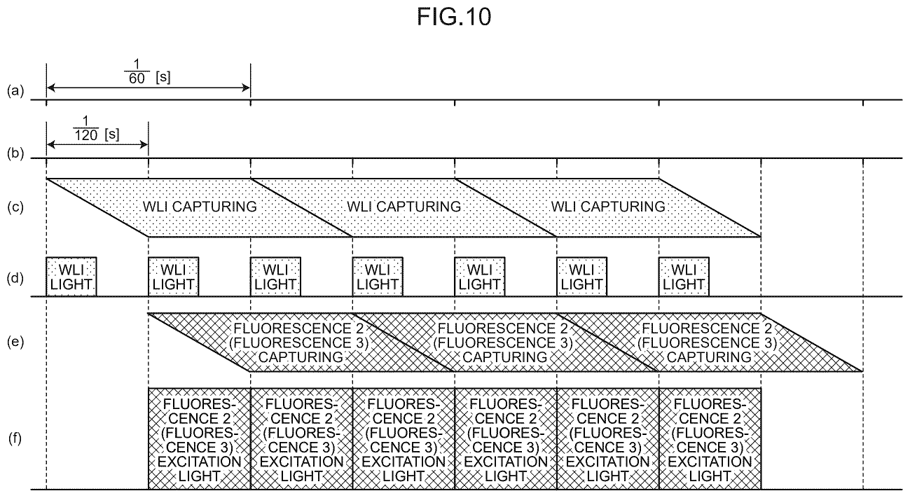

FIG. 10 is a diagram illustrating the operations of the second imaging unit 52 and the light source device 3 in the second and third modes. Specifically, (a) to (d) of FIG. 10 are diagrams corresponding to (a) to (d) of FIG. 7, respectively. (e) of FIG. 10 is a diagram corresponding to (e) of FIG. 7, in which the vertical axis represents a horizontal line of the fourth image sensor 524 (the uppermost stage represents the uppermost horizontal line (first horizontal line), and the lowermost stage represents the lowermost horizontal line (final line)), and the horizontal axis represents the time. A parallelogram region is a region contributing to the generation of the second fluorescence observation image or the third fluorescence observation image in one frame (field). For convenience of description, in (e) of FIG. 10, a text “Fluorescence 2 (Fluorescence 3) Capturing” representing the capturing of the fluorescence LF2 (second fluorescence or third fluorescence) is illustrated in the parallelogram regions. (f) of FIG. 10 is a diagram corresponding to (f) of FIG. 7, in which the vertical axis represents a power value [W] that is supplied to the third light source 33 or the fourth light source 34, and the horizontal axis represents the time (the supply time of the power supplied to the third light source 33 or the fourth light source 34). In the present embodiment, a voltage value that is supplied to the third light source 33 or the fourth light source 34 is fixed. Therefore, in (f) of FIG. 10, the vertical axis corresponds to a current value that is supplied to the third light source 33 or the fourth light source 34.

When set to the second and third modes, the control unit 94 controls the operations of the second imaging unit 52 and the light source device 3 as described below.

As illustrated in (c) of FIG. 10, the control unit 94 executes a capturing control of the third image sensor 523 as in the case where the control unit 94 is set to the first mode.

Further, as illustrated in (e) of FIG. 10, as in the capturing control of the third image sensor 523, the control unit 94 executes a capturing control using a rolling shutter system for the fourth image sensor 524. At this time, read timings in the respective fields of the third and fourth image sensors 523 and 524 are shifted from each other by 1/120 [s] as illustrated in (c) and (e) of FIG. 10. The read timings of the respective fields may be shifted from each other or may be the same.

Further, as illustrated in (f) of FIG. 10, the control unit 94 constantly supplies power to the third light source 33 or the fourth light source 34. As a result, the third light source 33 or the fourth light source 34 is constantly turned on to constantly emit the second excitation light or the third excitation light. In (f) of FIG. 10, for convenience of description, a text “Fluorescence 2 (Fluorescence 3) Excitation Light” representing emission of the excitation light LE2 (the second excitation light or the third excitation light) is illustrated in rectangular regions representing the supply state of power.

Through the operation of the second imaging unit 52 and the operation of the light source device 3 described above, the normal observation image for left eye and the second fluorescence observation image (fluorescence observation light for left eye) or the third fluorescence observation image (fluorescence observation light for left eye) are generated.

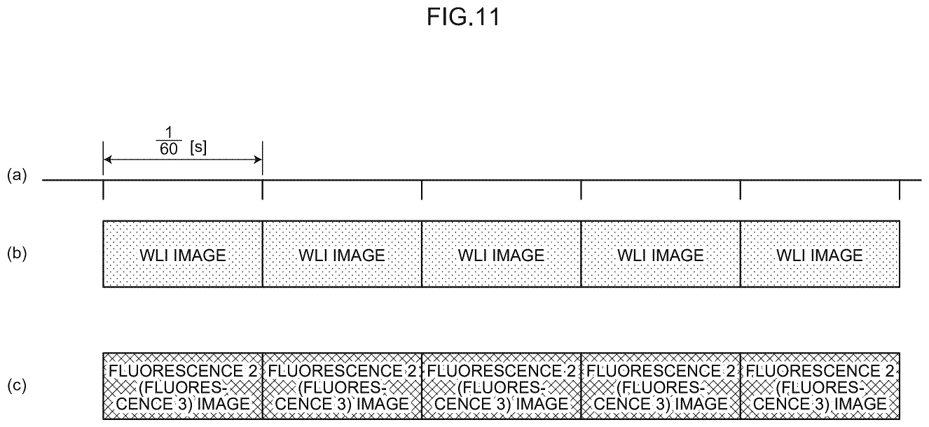

FIG. 11 is a diagram illustrating an updated image of the captured image generated in the second and third modes. Specifically, (a) of FIG. 11 is a diagram corresponding to (a) of FIG. 8. (b) of FIG. 11 is a diagram corresponding to (b) of FIG. 8. (c) of FIG. 11 is a diagram illustrating the updated image of the second fluorescence observation image or the third fluorescence observation image. For convenience of description, in (c) of FIG. 11, a text “Fluorescence 2 (Fluorescence 3) Image” representing the second fluorescence observation image or the third fluorescence observation image is illustrated in rectangular regions representing the second fluorescence observation image or the third fluorescence observation image.

Through the operations of the first and second imaging units 51 and 52 and the operation of the light source device 3 described above, each of the normal observation image for right eye, the normal observation image for left eye, and the second fluorescence observation image (fluorescence observation light for left eye) or the third fluorescence observation image (fluorescence observation light for left eye) that are generated is updated for each frame (period of 1/60 [s]) as illustrated in FIG. 11.

The fourth mode is a mode of generating the normal observation image for right eye, the normal observation image for left eye, the first fluorescence observation image, and the second fluorescence observation image or the third fluorescence observation image.

Hereinafter, operations of the first imaging unit 51 and the light source device 3 in the fourth mode, operations of the second imaging unit 52 and the light source device 3 in the fourth mode, and an updated image of the captured image generated in the fourth mode will be sequentially described.

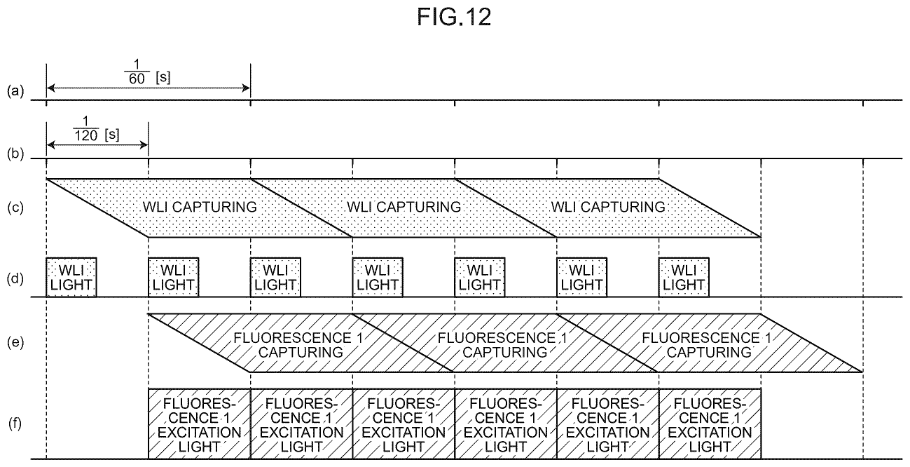

FIG. 12 is a diagram illustrating the operations of the first imaging unit 51 and the light source device 3 in the fourth mode. Specifically, (a) to (f) of FIG. 12 are diagrams corresponding to (a) to (f) of FIG. 6, respectively.

When set to the fourth mode, the control unit 94 controls the operations of the first imaging unit 51 and the light source device 3 as described below.

As illustrated in (c) of FIG. 12, the control unit 94 executes a capturing control of the first image sensor 513 as in the case where the control unit 94 is set to the first mode. In addition, as illustrated in (d) of FIG. 12, the control unit 94 executes a light source control of the first light source 31 as in the case where the control unit 94 is set to the first mode. Further, as illustrated in (e) of FIG. 12, the control unit 94 executes a capturing control of the second image sensor 514 as in the case where the control unit 94 is set to the first mode. In addition, as illustrated in (f) of FIG. 12, the control unit 94 executes a light source control to the second light source 32.

Through the operation of the first imaging unit 51 and the operation of the light source device 3 described above, the normal observation image for right eye and the first fluorescence observation image (fluorescence observation light for right eye) are generated.

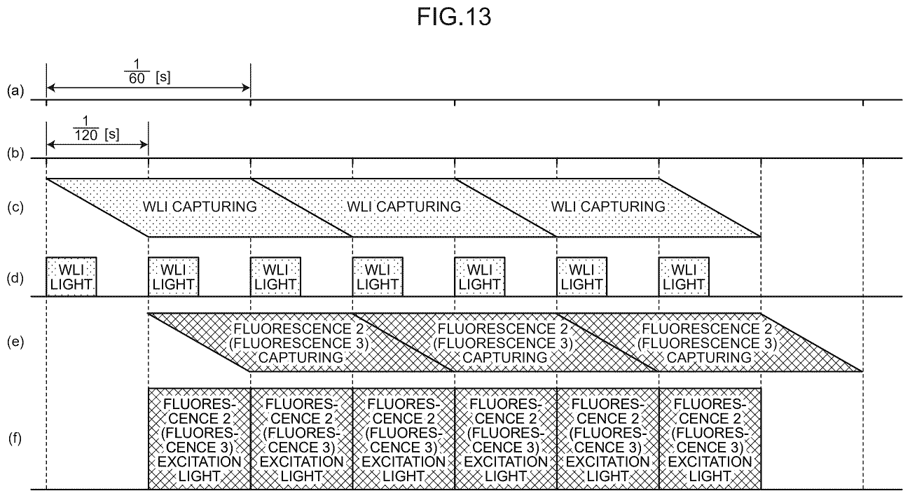

FIG. 13 is a diagram illustrating the operations of the second imaging unit 52 and the light source device 3 in the fourth mode. Specifically, (a) to (f) of FIG. 13 are diagrams corresponding to (a) to (f) of FIG. 10, respectively.

When set to the fourth mode, the control unit 94 controls the operations of the second imaging unit 52 and the light source device 3 as described below.

As illustrated in (c) of FIG. 13, the control unit 94 executes a capturing control of the third image sensor 523 as in the case where the control unit 94 is set to the second and third modes.

In addition, as illustrated in (e) of FIG. 13, the control unit 94 executes a capturing control of the fourth image sensor 524 as in the case where the control unit 94 is set to the second and third modes.

In addition, as illustrated in (f) of FIG. 13, the control unit 94 executes a light source control of the third light source 33 or the fourth light source 34 as in the case where the control unit 94 is set to the second and third modes.

Through the operation of the second imaging unit 52 and the operation of the light source device 3 described above, the normal observation image for left eye and the second fluorescence observation image (fluorescence observation light for left eye) or the third fluorescence observation image (fluorescence observation light for left eye) are generated.

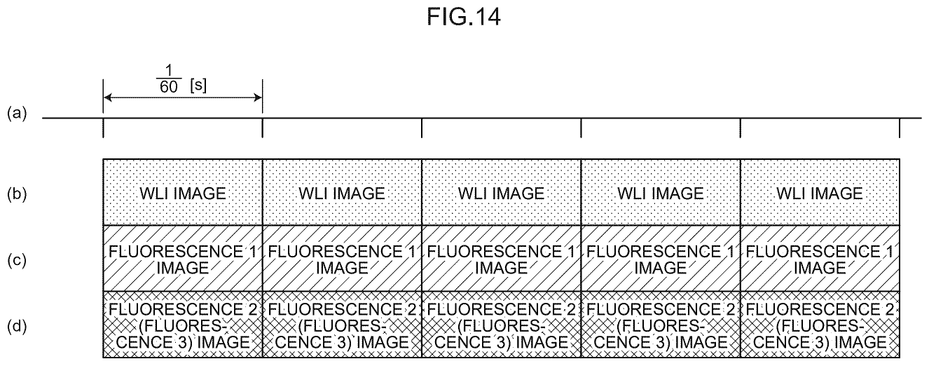

FIG. 14 is a diagram illustrating the updated image of the captured image generated in the fourth mode. Specifically, (a) to (c) of FIG. 14 are diagrams corresponding to (a) to (c) of FIG. 8, respectively. (d) of FIG. 14 is a diagram corresponding to (c) of FIG. 11.

Through the operations of the first and second imaging units 51 and 52 and the operation of the light source device 3 described above, each of the normal observation image for right eye, the normal observation image for left eye, the first fluorescence observation image (fluorescence observation light for right eye), and the second fluorescence observation image (fluorescence observation light for left eye) or the third fluorescence observation image (fluorescence observation light for left eye) that are generated is updated for each frame (period of 1/60 [s]) as illustrated in FIG. 14.

With the present embodiment described above, the following effects are exhibited.

The camera head 5 according to the present embodiment includes: the first imaging unit 51 configured to capture the normal observation light for right eye and the first fluorescence; and the second imaging unit 52 configured to capture the normal observation light for left eye and the second and third fluorescence components. That is, the first and second imaging units 51 and 52 do not have the same configuration, the first imaging unit 51 captures at least one of two or more types of fluorescence, and the second imaging unit 52 captures at least the other one of the two or more types of fluorescence. Therefore, with the camera head 5 according to the present embodiment, miniaturization can be achieved while enabling the return light of the white light and two or more types of fluorescence to be captured.

In addition, the image sensors 514 and 524 that capture the fluorescence and the image sensors 513 and 523 that capture the white light are separately provided. Therefore, the sensitivity to the fluorescence in the image sensors 514 and 524 can be enhanced.

Here, the embodiment of the present disclosure has been described. However, the present disclosure is not limited to only the above-described embodiment.

In the above-described embodiment, Modification Examples 1 to 9 described below may also be adopted.

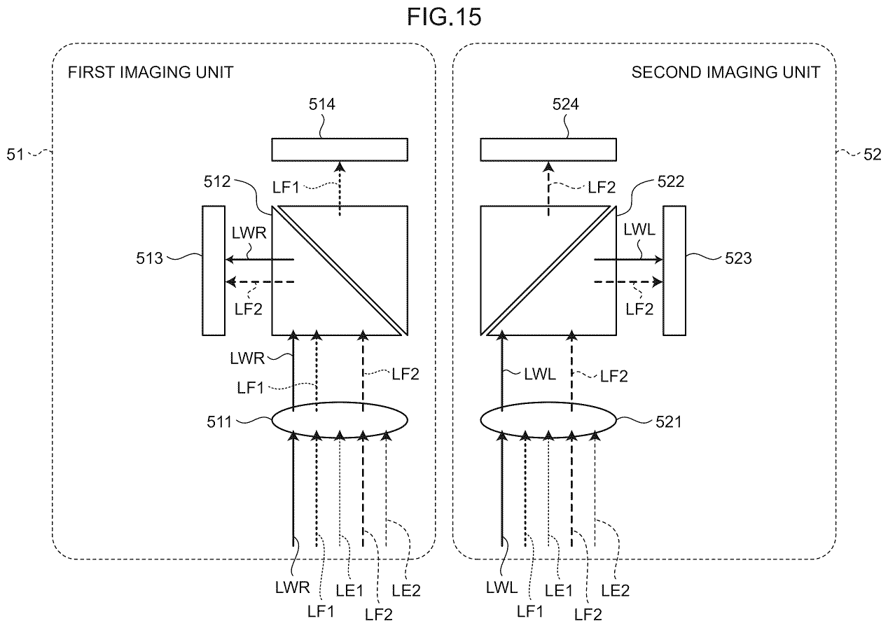

FIG. 15 is a diagram corresponding to FIG. 3 and illustrating a configuration of the first and second imaging units 51 and 52 according to Modification Example 1 of the above-described embodiment.

In Modification Example 1, the first to fourth optical members 511, 512, 521, and 522 are configured as described below.

As illustrated in FIG. 15, the first optical member 511 according to Modification Example 1 has a light cutting function of cutting the excitation light components LE1 and LE2 in the light that is focused in the insertion unit 2 and is incident on the first optical member 511. The light that is focused in the insertion unit 2 and is incident on the first optical member 511 includes the normal observation light for right eye LWR, the excitation light components LE1 and LE2, and the fluorescence components LF1 and LF2.

As illustrated in FIG. 15, the second optical member 512 according to Modification Example 1 has a light separating mechanism of separating the normal observation light for right eye LWR, the fluorescence LF2, and the fluorescence LF1 that are not cut by the first optical member 511. The second optical member 512 allows the normal observation light for right eye LWR and the fluorescence LF2 to travel toward the first image sensor 513, and allows the fluorescence LF1 to travel toward the second image sensor 514.

As illustrated in FIG. 15, the third optical member 521 according to Modification Example 1 has the same function as the third optical member 521 described in the above-described embodiment.

As illustrated in FIG. 15, the fourth optical member 522 according to Modification Example 1 has a light separating function of separating a part of the fluorescence LF2 and the normal observation light for left eye LWL and the other fluorescence LF2 that is not cut by the third optical member 521. The fourth optical member 522 allows a part of the fluorescence LF2 and the normal observation light for left eye LWL to travel toward the third image sensor 523, and allows the other fluorescence LF2 to travel toward the fourth image sensor 524.

In Modification Example 1, a correspondence between the first to fourth image sensors 513, 514, 523, and 524, the white light, and the first to third fluorescence components is as shown in Table 2 below.

TABLE 2

| | First | Second | Third | Fourth |
|---|---|---|---|---|
| | image | image | image | image |
| | sensor | sensor | sensor | sensor |
| White light | ⊚ | X | ⊚ | X |
| First | X | ◯ | X | X |
| fluorescence | | | | |
| Second | X | X | X | ◯ |
| fluorescence | | | | |
| Third | ⊚ | X | ⊚ | (⊚) |
| fluorescence | | | | |

In Table 2, “⊚” and “(⊚)” represent being capturable three-dimensionally (in 3D). That is, a 3D image can be generated by the normal observation image for right eye generated in the first image sensor 513 and the normal observation image for left eye generated in the third image sensor 523. In addition, a 3D image can be generated by the third fluorescence observation image (first stereoscopic fluorescence observation image (fluorescence observation light for right eye (first fluorescence observation light))) generated by the first image sensor 513 and the third fluorescence observation image (second stereoscopic fluorescence observation image (fluorescence observation light for left eye (second fluorescence observation light))) generated by the third image sensor 523. Further, a 3D image can be generated by the third fluorescence observation image (first stereoscopic fluorescence observation image (fluorescence observation light for right eye (first fluorescence observation light))) generated by the first image sensor 513 and the third fluorescence observation image (second stereoscopic fluorescence observation image (fluorescence observation light for left eye (second fluorescence observation light))) generated by the fourth image sensor 524. In addition, “◯” represents being capturable two-dimensionally (in 2D). That is, the 2D first fluorescence observation image can be generated by the second image sensor 514. In addition, the 2D second fluorescence observation image can be generated by the fourth image sensor 524. Further, “X” represents not being supported.

An operation corresponding to the first mode according to Modification Example 1 is the same as the operation corresponding to the first mode described in the above-described embodiment. In addition, an operation corresponding to the second mode according to Modification Example 1 is the same as the operations corresponding to the second and third modes described in the above-described embodiment. Therefore, hereinafter, an operation corresponding to the third mode and an operation corresponding to the fourth mode according to Modification Example 1 will be sequentially described.

Hereinafter, operations of the first imaging unit 51 and the light source device 3 in the third mode, operations of the second imaging unit 52 and the light source device 3 in the third mode, and an updated image of the captured image generated in the third mode will be sequentially described.

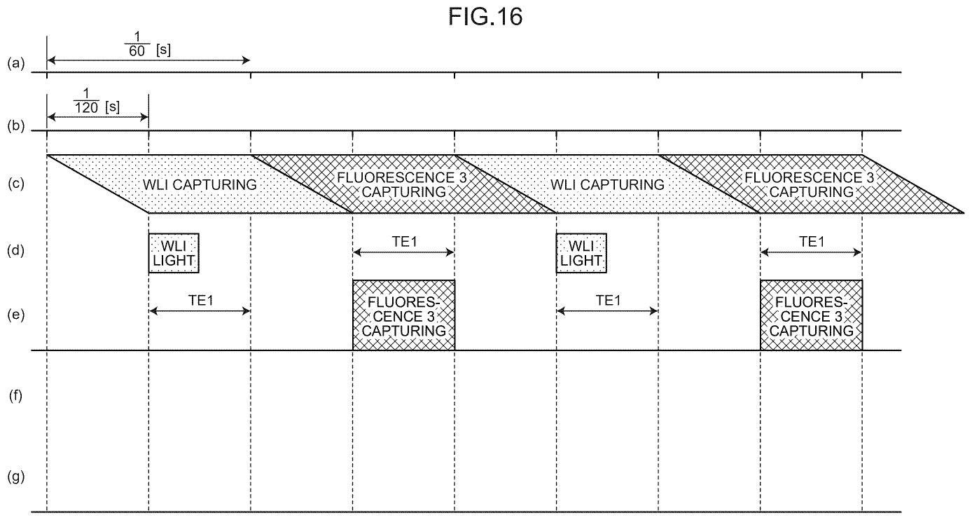

FIG. 16 is a diagram illustrating the operations of the first imaging unit 51 and the light source device 3 in the third mode. Specifically, (a) to (d), (f), and (g) of FIG. 16 are diagrams corresponding to (a) to (f) of FIG. 6, respectively. For convenience of description, in (c) of FIG. 16, a text “Fluorescence 3 Capturing” representing the capturing of the third fluorescence observation image is illustrated in parallelogram regions contributing to the generation of the third fluorescence observation image. (e) of FIG. 16 is a diagram corresponding to (f) of FIG. 10, in which the vertical axis represents a power value [W] that is supplied to the fourth light source 34, and the horizontal axis represents the time (the supply time of the power supplied to the fourth light source 34). In the present embodiment, a voltage value that is supplied to the fourth light source 34 is fixed. Therefore, in (e) of FIG. 16, the vertical axis corresponds to a current value that is supplied to the fourth light source 34. In addition, In (e) of FIG. 16, for convenience of description, a text “Fluorescence 3 Excitation Light” representing emission of the third excitation light is illustrated in rectangular regions representing the supply state of power to the fourth light source 34.

When set to the third mode, the control unit 94 controls the operations of the first imaging unit 51 and the light source device 3 as described below.

As illustrated in (c) of FIG. 16, the control unit 94 executes a capturing control of the first image sensor 513 as in the case where the control unit 94 is set to the first mode.

In addition, as illustrated in (d) and (e) of FIG. 16, in an all-line exposure period TE1 for each frame (period of 1/60 [s]) in the first image sensor 513, the control unit 94 alternately repeats the emission of the white light from the first light source 31 and the emission of the third excitation light from the fourth light source 34. Here, the supply time of the power supplied to the first light source 31 is shorter than the all-line exposure period TE1 ( 1/120 [s]). As a result, the first light source 31 emits the white light for a time of shorter than the all-line exposure period TE1 ( 1/120 [s]). On the other hand, the supply time of the power supplied to the third light source 33 is the all-line exposure period TE1. As a result, the third light source 33 emits the third excitation light for a time of the all-line exposure period TE1 ( 1/120 [s]). Therefore, as illustrated in (c) of FIG. 16 the first image sensor 513 captures the white light and the third fluorescence in a time division manner for each frame.

Through the operation of the first imaging unit 51 and the operation of the light source device 3 described above, the normal observation image for right eye and the third fluorescence observation image (first stereoscopic fluorescence observation image (fluorescence observation light for right eye (first fluorescence observation light))) are generated.

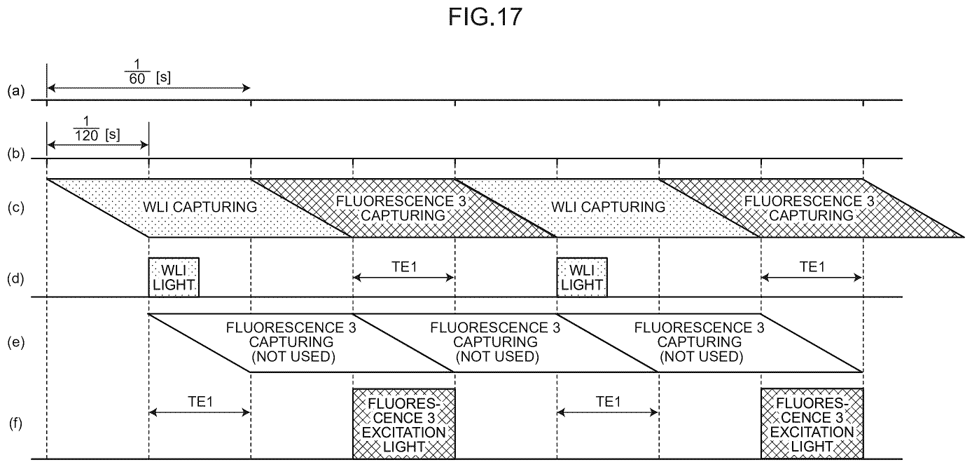

FIG. 17 is a diagram illustrating the operations of the second imaging unit 52 and the light source device 3 in the third mode. Specifically, (a) to (e) of FIG. 17 are diagrams corresponding to (a) to (e) of FIG. 10, respectively. (f) of FIG. 17 is a diagram corresponding to (e) of FIG. 16.

When set to the third mode, the control unit 94 controls the operations of the second imaging unit 52 and the light source device 3 as described below.

As illustrated in (c) of FIG. 17, the control unit 94 executes a capturing control of the third image sensor 523 as in the case where the control unit 94 is set to the first mode. Therefore, the third image sensor 523 captures the white light and the third fluorescence in a time division manner for each frame. As illustrated in (e) of FIG. 17, the control unit 94 does not operate the fourth image sensor 524 (even if operating the fourth image sensor 524, the captured third fluorescence observation image is not used).

Through the operation of the second imaging unit 52 and the operation of the light source device 3 described above, the normal observation image for left eye and the third fluorescence observation image (second stereoscopic fluorescence observation image (fluorescence observation light for left eye (second fluorescence observation light))) are generated.

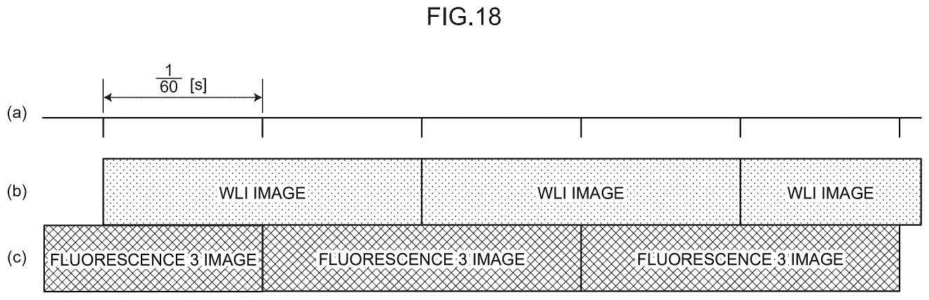

FIG. 18 is a diagram illustrating the updated image of the captured image generated in the third mode. Specifically, (a) and (b) of FIG. 18 are diagrams corresponding to (a) and (b) of FIG. 8, respectively. (c) of FIG. 18 is a diagram corresponding to (c) of FIG. 11 and illustrating the updated image of the third fluorescence observation image. For convenience of description, in (c) of FIG. 18, a text “Fluorescence 3 Image” representing the third fluorescence observation image is illustrated in rectangular regions representing the third fluorescence observation image.

Through the operations of the first and second imaging units 51 and 52 and the operation of the light source device 3 described above, each of the normal observation image for right eye, the normal observation image for left eye, and the third fluorescence observation image (fluorescence observation light for right eye, fluorescence observation light for left eye) that are generated is updated for every two frames (period of 1/30 [s]) as illustrated in FIG. 18.

Hereinafter, operations of the first imaging unit 51 and the light source device 3 in the fourth mode, operations of the second imaging unit 52 and the light source device 3 in the fourth mode, and an updated image of the captured image generated in the fourth mode will be sequentially described.

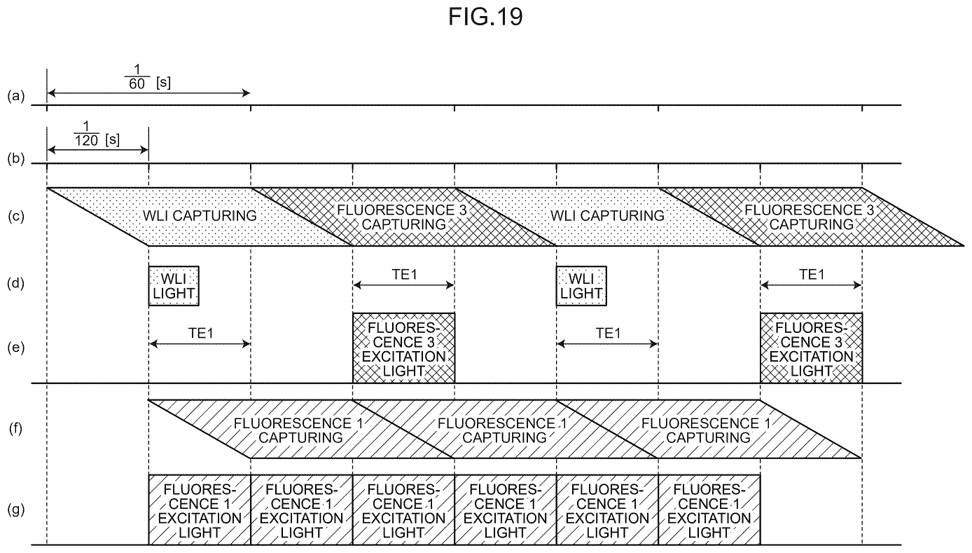

FIG. 19 is a diagram illustrating the operations of the first imaging unit 51 and the light source device 3 in the fourth mode. Specifically, (a) to (g) of FIG. 19 are diagrams corresponding to (a) to (g) of FIG. 16, respectively.

When set to the fourth mode, the control unit 94 controls the operations of the first imaging unit 51 and the light source device 3 as described below.

As illustrated in (c) of FIG. 19, the control unit 94 executes a capturing control of the first image sensor 513 as in the case where the control unit 94 is set to the third mode. In addition, as illustrated in (d) and (e) of FIG. 19, the control unit 94 executes a light source control of the first and third light sources 31 and 33 as in the case where the control unit 94 is set to the third mode. As a result, as illustrated in (c) of FIG. 19 the first image sensor 513 captures the white light and the third fluorescence in a time division manner for each frame.

In addition, as illustrated in (f) of FIG. 19, the control unit 94 executes a capturing control of the second image sensor 514 as in the case where the control unit 94 is set to the first mode.

Further, as illustrated in (g) of FIG. 19, the control unit 94 executes a light source control of the second light source 32 as in the case where the control unit 94 is set to the first mode.

Through the operations of the first imaging unit 51 and the light source device 3 described above, the normal observation image for right eye, the first fluorescence observation image (fluorescence observation light for right eye), and the third fluorescence observation image (first stereoscopic fluorescence observation image (fluorescence observation light for right eye (first fluorescence observation light))) are generated.

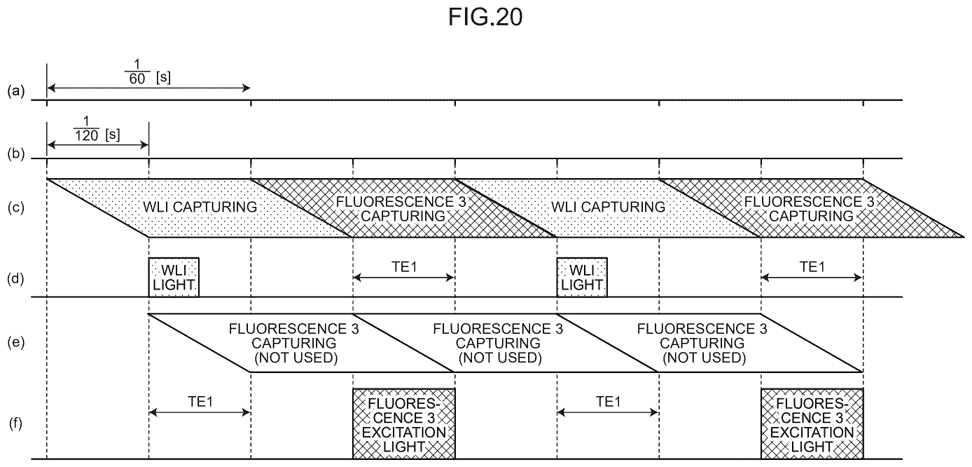

FIG. 20 is a diagram illustrating the operations of the second imaging unit 52 and the light source device 3 in the fourth mode. Specifically, (a) to (f) of FIG. 20 are diagrams corresponding to (a) to (f) of FIG. 17, respectively.

When set to the fourth mode, the control unit 94 controls the operations of the second imaging unit 52 and the light source device 3 as illustrated in FIG. 20 as in the case where the control unit 94 is set to the third mode.

Through the operation of the second imaging unit 52 and the operation of the light source device 3 described above, the normal observation image for left eye and the third fluorescence observation image (second stereoscopic fluorescence observation image (fluorescence observation light for left eye (second fluorescence observation light))) are generated.

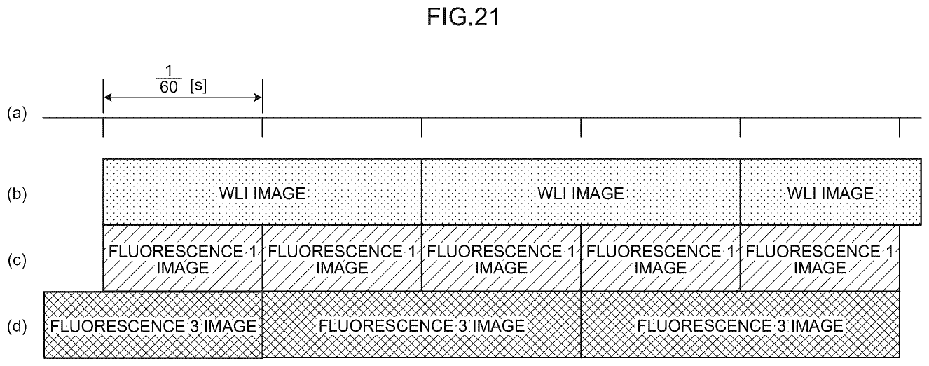

FIG. 21 is a diagram illustrating the updated image of the captured image generated in the fourth mode. Specifically, (a), (b), and (d) of FIG. 21 are diagrams corresponding to (a) to (c) of FIG. 18, respectively. (c) of FIG. 21 is a diagram corresponding to (c) of FIG. 8.

Through the operations of the first and second imaging units 51 and 52 and the operation of the light source device 3 described above, each of the normal observation image for right eye, the normal observation image for left eye, and the third fluorescence observation image (fluorescence observation light for right eye, fluorescence observation light for left eye) that are generated is updated for every two frames (period of 1/30 [s]) as illustrated in FIG. 21. In addition, the generated first fluorescence observation image (fluorescence observation light for right eye) is updated for each frame (period of 1/60 [s]).

Even with the configuration of Modification Example 1 described above, the same effects as in the above-described embodiment are exhibited.

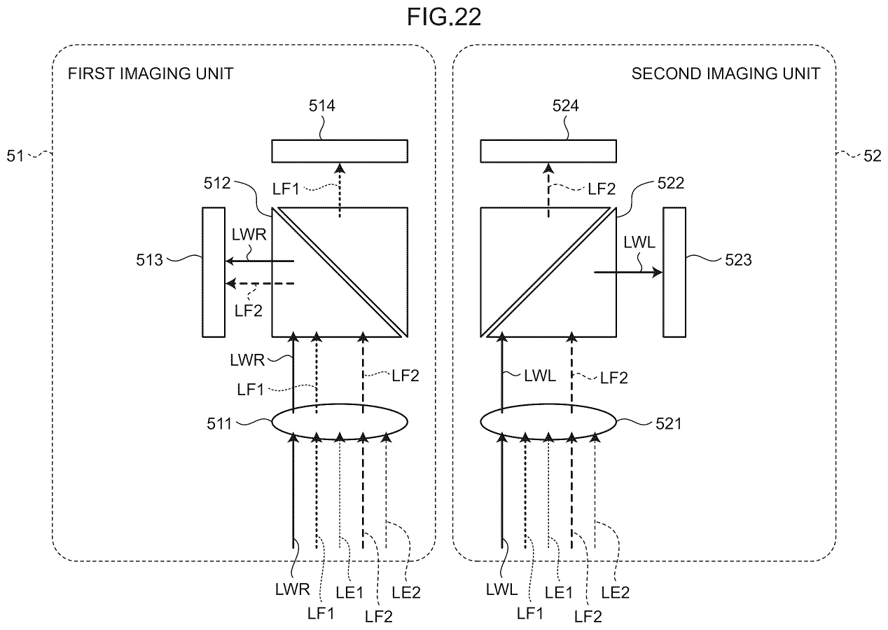

FIG. 22 is a diagram corresponding to FIG. 3 and illustrating a configuration of the first and second imaging units 51 and 52 according to Modification Example 2 of the above-described embodiment.

In Modification Example 2, the first to fourth optical members 511, 512, 521, and 522 are configured as described below.

As illustrated in FIG. 22, the first to third optical members 511, 512, and 521 according to Modification Example 2 have the same functions as the first to third optical members 511, 512, and 521 described above in Modification Example 1, respectively. In addition, the fourth optical member 524 according to Modification Example 2 has the same function as the fourth optical member 524 described above in the embodiment.

In Modification Example 2, a correspondence between the first to fourth image sensors 513, 514, 523, and 524, the white light, and the first to third fluorescence components is as shown in Table 3 below.

TABLE 3

| | First | Second | Third | Fourth |
|---|---|---|---|---|
| | image | image | image | image |
| | sensor | sensor | sensor | sensor |
| White light | ⊚ | X | ⊚ | X |
| First | X | ◯ | X | X |
| fluorescence | | | | |
| Second | X | X | X | ◯ |
| fluorescence | | | | |
| Third | ⊚ | X | X | ⊚ |
| fluorescence | | | | |

In Table 3, “⊚” represents being capturable in 3D. That is, a 3D image can be generated by the normal observation image for right eye generated in the first image sensor 513 and the normal observation image for left eye generated in the third image sensor 523. In addition, a 3D image can be generated by the third fluorescence observation image (first stereoscopic fluorescence observation image (fluorescence observation light for right eye (first fluorescence observation light))) generated by the first image sensor 513 and the third fluorescence observation image (second stereoscopic fluorescence observation image (fluorescence observation light for left eye (second fluorescence observation light))) generated by the fourth image sensor 524. In addition, “◯” represents being capturable two-dimensionally (in 2D). That is, the 2D first fluorescence observation image can be generated by the second image sensor 514. In addition, the 2D second fluorescence observation image can be generated by the fourth image sensor 524. Further, “X” represents not being supported.

An operation corresponding to the first mode according to Modification Example 2 is the same as the operation corresponding to the first mode described in the above-described embodiment. In addition, an operation corresponding to the second mode according to Modification Example 2 is the same as the operations corresponding to the second and third modes described in the above-described embodiment. Therefore, hereinafter, an operation corresponding to the third mode and an operation corresponding to the fourth mode according to Modification Example 2 will be sequentially described.

Hereinafter, operations of the first imaging unit 51 and the light source device 3 in the third mode, operations of the second imaging unit 52 and the light source device 3 in the third mode, and an updated image of the captured image generated in the third mode will be sequentially described.

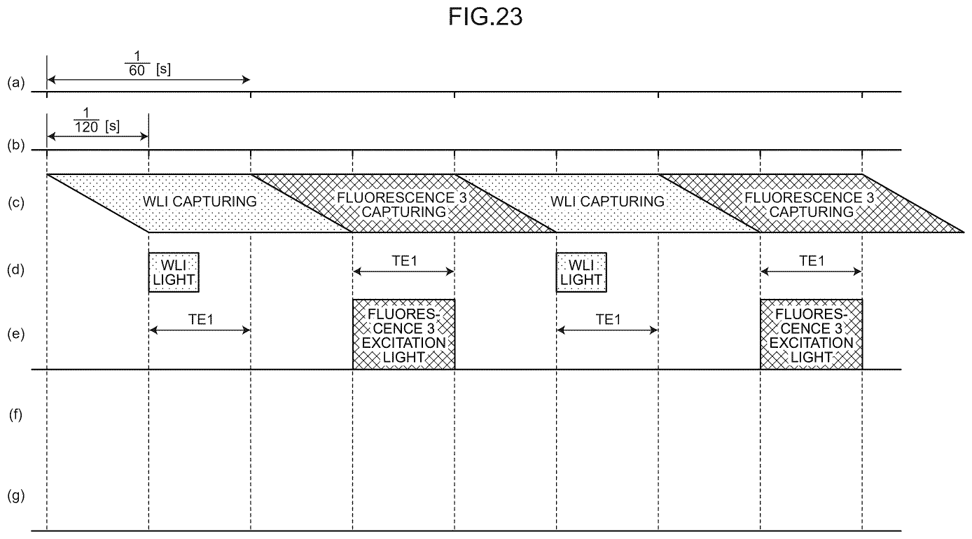

FIG. 23 is a diagram illustrating the operations of the first imaging unit 51 and the light source device 3 in the third mode. Specifically, (a) to (g) of FIG. 23 are diagrams corresponding to (a) to (g) of FIG. 16, respectively.

When set to the third mode, the control unit 94 controls the operations of the first imaging unit 51 and the light source device 3 as illustrated in FIG. 23 as in the case where the control unit 94 is set to the third mode in Modification Example 1 above.

Through the operations of the first imaging unit 51 and the light source device 3 described above, the normal observation image for right eye and the third fluorescence observation image (first stereoscopic fluorescence observation image (fluorescence observation light for right eye (first fluorescence observation light))) are generated.

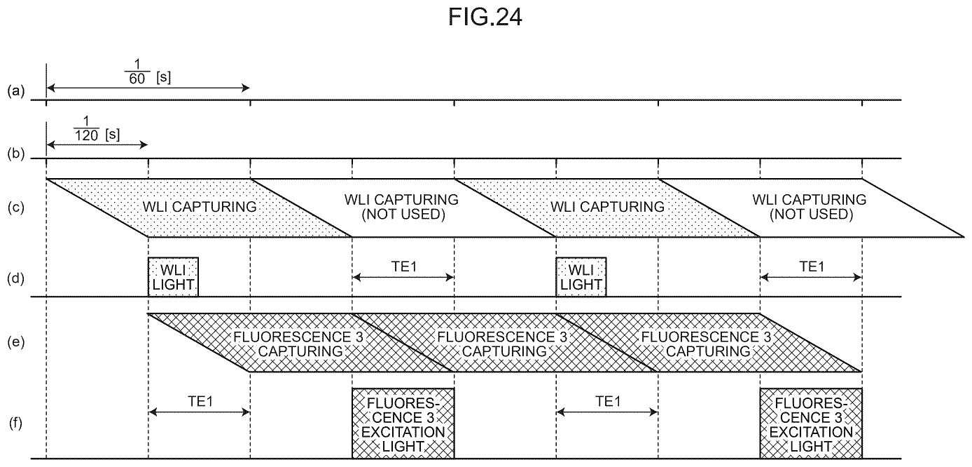

FIG. 24 is a diagram illustrating the operations of the second imaging unit 52 and the light source device 3 in the third mode. Specifically, (a) to (f) of FIG. 24 are diagrams corresponding to (a) to (f) of FIG. 17, respectively.

When set to the third mode, the control unit 94 controls the operations of the second imaging unit 52 and the light source device 3 as described below.

As illustrated in (c) of FIG. 24, the control unit 94 executes a capturing control of the third image sensor 523 as in the case where the control unit 94 is set to the first mode. Therefore, the third image sensor 523 captures the white light in a time division manner for each frame. Here, the normal observation image for left eye that is captured in a period where the third fluorescence is emitted is not used.

In addition, as illustrated in (e) of FIG. 24, the control unit 94 executes a capturing control of the fourth image sensor 524 as in the case where the control unit 94 is set to the second mode.

Through the operation of the second imaging unit 52 and the operation of the light source device 3 described above, the normal observation image for left eye and the third fluorescence observation image (second stereoscopic fluorescence observation image (fluorescence observation light for left eye (second fluorescence observation light))) are generated.

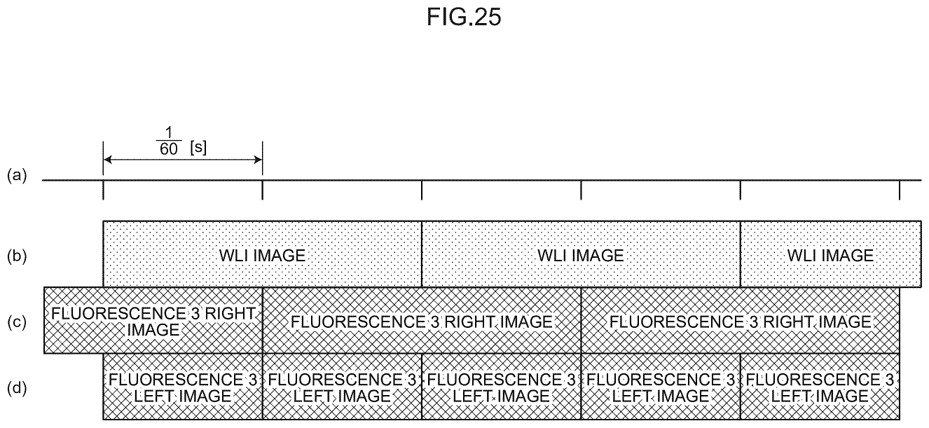

FIG. 25 is a diagram illustrating the updated image of the captured image generated in the third mode. Specifically, (a) and (b) of FIG. 25 are diagrams corresponding to (a) and (b) of FIG. 8, respectively. (c) of FIG. 25 is a diagram illustrating the updated image of the third fluorescence observation image (fluorescence observation light for right eye). In (c) of FIG. 25, for convenience of description, a text “Fluorescence 3 Right Image” representing the third fluorescence observation image (first stereoscopic fluorescence observation image (fluorescence observation light for right eye (first fluorescence observation light))) is illustrated in rectangular regions representing the third fluorescence observation image (first stereoscopic fluorescence observation image (fluorescence observation light for right eye (first fluorescence observation light))). (d) of FIG. 25 is a diagram illustrating the updated image of the third fluorescence observation image (fluorescence observation light for left eye). In (d) of FIG. 25, for convenience of description, a text “Fluorescence 3 Left Image” representing the third fluorescence observation image (second stereoscopic fluorescence observation image (fluorescence observation light for left eye)) is illustrated in rectangular regions representing the third fluorescence observation image (second stereoscopic fluorescence observation image (fluorescence observation light for left eye (second fluorescence observation light))).

Through the operations of the first and second imaging units 51 and 52 and the operation of the light source device 3 described above, each of the normal observation image for right eye, the normal observation image for left eye, and the third fluorescence observation image (first stereoscopic fluorescence observation image (fluorescence observation light for right eye (first fluorescence observation light))) that are generated is updated for every two frames (period of 1/30 [s]) as illustrated in FIG. 25. In addition, the third fluorescence observation image (second stereoscopic fluorescence observation image (fluorescence observation light for left eye (second fluorescence observation light))) that is generated is updated for each frame (period of 1/60 [s]).

Hereinafter, operations of the first imaging unit 51 and the light source device 3 in the fourth mode, operations of the second imaging unit 52 and the light source device 3 in the fourth mode, and an updated image of the captured image generated in the fourth mode will be sequentially described.

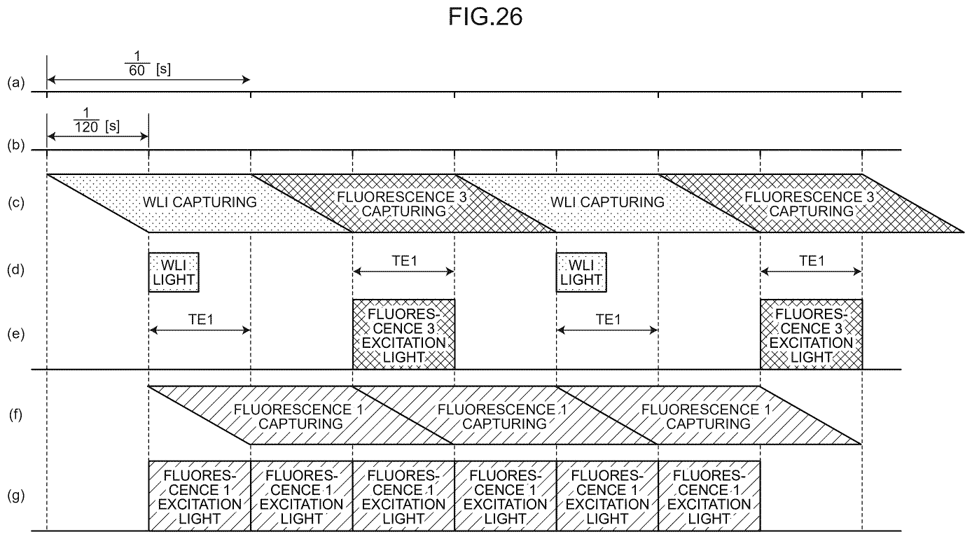

FIG. 26 is a diagram illustrating the operations of the first imaging unit 51 and the light source device 3 in the fourth mode. Specifically, (a) to (g) of FIG. 26 are diagrams corresponding to (a) to (f) of FIG. 23, respectively.

When set to the fourth mode, the control unit 94 controls the operations of the first imaging unit 51 and the light source device 3 as in the case where the control unit 94 is set to the fourth mode in Modification Example 1 above.

Through the operations of the first imaging unit 51 and the light source device 3 described above, the normal observation image for right eye, the first fluorescence observation image (fluorescence observation light for right eye), and the third fluorescence observation image (first stereoscopic fluorescence observation image (fluorescence observation light for right eye (first fluorescence observation light))) are generated.

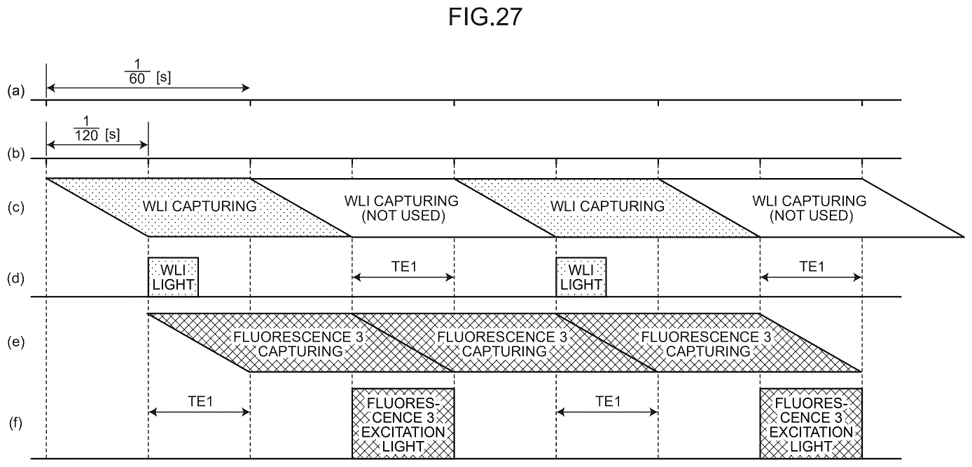

FIG. 27 is a diagram illustrating the operations of the second imaging unit 52 and the light source device 3 in the fourth mode. Specifically, (a) to (f) of FIG. 27 are diagrams corresponding to (a) to (f) of FIG. 24, respectively.

When set to the fourth mode, the control unit 94 controls the operations of the second imaging unit 52 and the light source device 3 as in the case where the control unit 94 is set to the third mode.

Through the operation of the second imaging unit 52 and the operation of the light source device 3 described above, the normal observation image for left eye and the third fluorescence observation image (second stereoscopic fluorescence observation image (fluorescence observation light for left eye (second fluorescence observation light))) are generated.

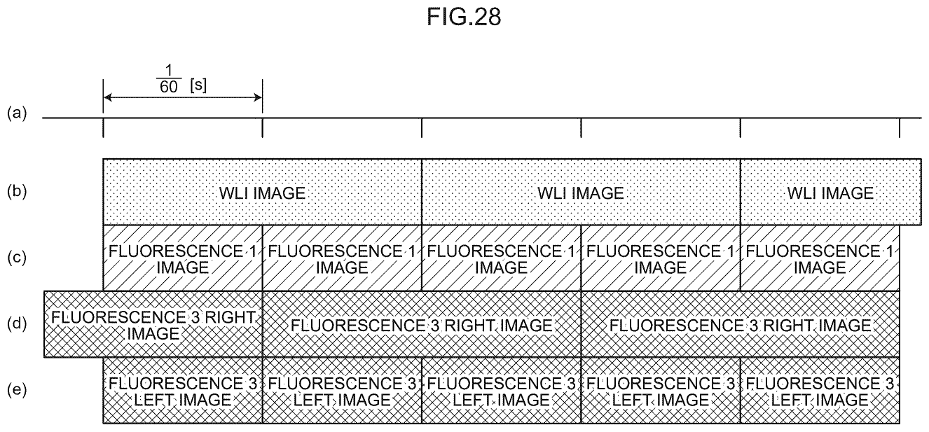

FIG. 28 is a diagram illustrating the updated image of the captured image generated in the fourth mode. Specifically, (a), (b), (d), and (e) of FIG. 28 are diagrams corresponding to (a) to (d) of FIG. 25, respectively. (c) of FIG. 28 is a diagram corresponding to (c) of FIG. 8.

Through the operations of the first and second imaging units 51 and 52 and the operation of the light source device 3 described above, each of the normal observation image for right eye, the normal observation image for left eye, and the third fluorescence observation image (first stereoscopic fluorescence observation image (fluorescence observation light for right eye (first fluorescence observation light))) that are generated is updated for every two frames (period of 1/30 [s]) as illustrated in FIG. 28. In addition, each of the first fluorescence observation image (fluorescence observation light for right eye) and the third fluorescence observation image (second stereoscopic fluorescence observation image (fluorescence observation light for left eye (second fluorescence observation light))) that are generated is updated for each frame (period of 1/60 [s]).

Even with the configuration of Modification Example 2 described above, the same effects as in the above-described embodiment are exhibited.

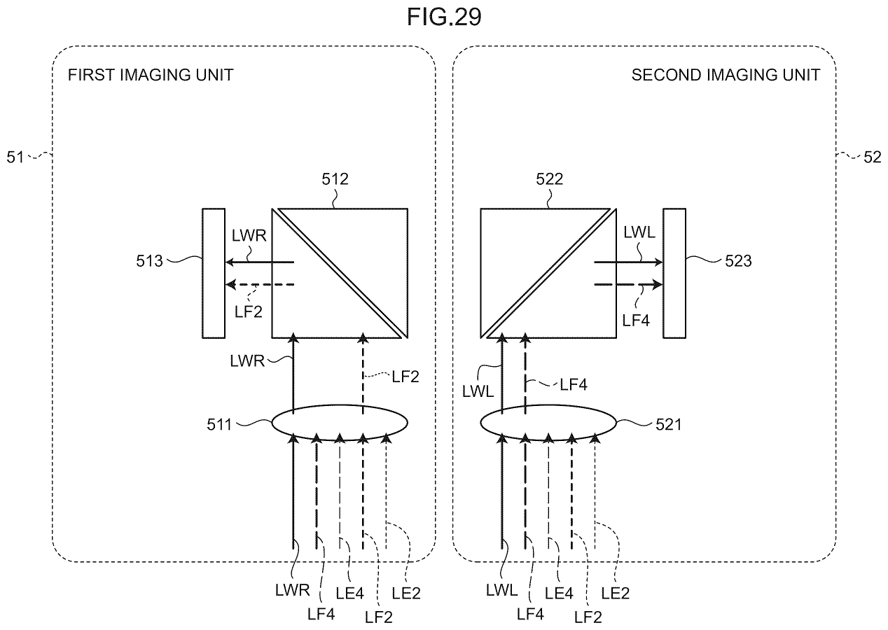

FIG. 29 is a diagram corresponding to FIG. 3 and illustrating a configuration of the first and second imaging units 51 and 52 according to Modification Example 3 of the above-described embodiment.

In Modification Example 3 the third light source 33 is not provided. In addition, the second light source 32 according to Modification Example 3 emits fourth excitation light having a different wavelength band from that of the first to third excitation light components. In addition, the return light of the fourth excitation light that is reflected from the observation target by irradiation of the observation target includes not only the fourth excitation light reflected from the observation target but also fluorescence (hereinafter, referred to as fourth fluorescence) that is emitted from a substance in the observation target when the substance is excited. Here, the fourth fluorescence and the first to third fluorescence components have different wavelength bands. In FIG. 29, the fourth excitation light is represented by excitation light LE4, and the fourth fluorescence is represented by fluorescence LF4. The fourth fluorescence corresponds to the first fluorescence according to the present disclosure.

In the first imaging unit 51 according to Modification Example 3, the second image sensor 514 described in the above-described embodiment is not provided. In addition, in the second imaging unit 52 according to Modification Example 3, the fourth image sensor 524 described in the above-described embodiment is not provided. Further, in Modification Example 3, the first to fourth optical members 511, 512, 521, and 522 are configured as described below.

As illustrated in FIG. 29, the first optical member 511 according to Modification Example 3 has a light cutting function of cutting the excitation light components LE2 and LE4 and the fluorescence LF4 in the light that is focused in the insertion unit 2 and is incident on the first optical member 511. The light that is focused in the insertion unit 2 and is incident on the first optical member 511 includes the normal observation light for right eye LWR, the excitation light components LE2 and LE4, and the fluorescence components LF2 and LF4.

As illustrated in FIG. 29, the second optical member 512 according to Modification Example 3 has a function of allowing the normal observation light for right eye LWR and the fluorescence LF2 that are not cut by the first optical member 511 to travel toward the first image sensor 513.

As illustrated in FIG. 29, the third optical member 521 according to Modification Example 3 has a light cutting function of cutting the excitation light components LE2 and LE4 and the fluorescence LF2 in the light that is focused in the insertion unit 2 and is incident on the third optical member 521. The light that is focused in the insertion unit 2 and is incident on the third optical member 521 includes the normal observation light for left eye LWL, the excitation light components LE2 and LE4, and the fluorescence components LF2 and LF4.

As illustrated in FIG. 29, the fourth optical member 522 according to Modification Example 3 has a function of allowing the normal observation light for left eye LWL and the fluorescence LF4 that are not cut by the third optical member 521 to travel toward the third image sensor 523.

In Modification Example 3, a correspondence between the first and third image sensors 513 and 523, the white light, and the first to fourth fluorescence components is as shown in Table 4 below.

TABLE 4

| | First | Third |
|---|---|---|
| | image sensor | image sensor |
| White light | ⊚ | ⊚ |
| First fluorescence | X | X |
| Second fluorescence | X | X |
| Third fluorescence | ◯ | X |
| Fourth fluorescence | X | ◯ |

In Table 4, “⊚” represents being capturable in 3D. That is, a 3D image can be generated by the normal observation image for right eye generated in the first image sensor 513 and the normal observation image for left eye generated in the third image sensor 523. In addition, “◯” represents being capturable two-dimensionally (in 2D). That is, the 2D third fluorescence observation image can be generated by the first image sensor 513. In addition, the 2D fourth fluorescence observation image can be generated by the third image sensor 523. The fourth fluorescence observation image refers to an image signal obtained by capturing the fourth fluorescence in the third image sensor 523. Further, “X” represents not being supported.

Hereinafter, an operation corresponding to the third mode and an operation corresponding to the fifth mode according to Modification Example 3 will be sequentially described.

Hereinafter, operations of the first imaging unit 51 and the light source device 3 in the third mode, operations of the second imaging unit 52 and the light source device 3 in the third mode, and an updated image of the captured image generated in the third mode will be sequentially described.

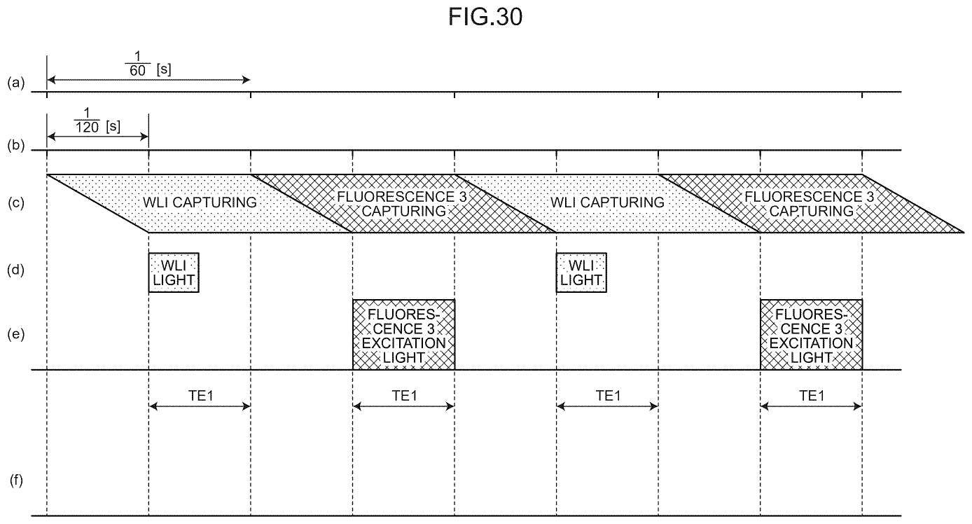

FIG. 30 is a diagram illustrating the operations of the first imaging unit 51 and the light source device 3 in the third mode. Specifically, (a) to (e) of FIG. 30 are diagrams corresponding to (a) to (e) of FIG. 23, respectively. (f) of FIG. 30 is a diagram corresponding to (f) of FIG. 6.

When set to the third mode, the control unit 94 controls the operations of the first imaging unit 51 and the light source device 3 as illustrated in FIG. 30 as in the case where the control unit 94 is set to the third mode in Modification Example 2 above.

Through the operation of the first imaging unit 51 and the operation of the light source device 3 described above, the normal observation image for right eye and the third fluorescence observation image (fluorescence observation light for right eye) are generated.

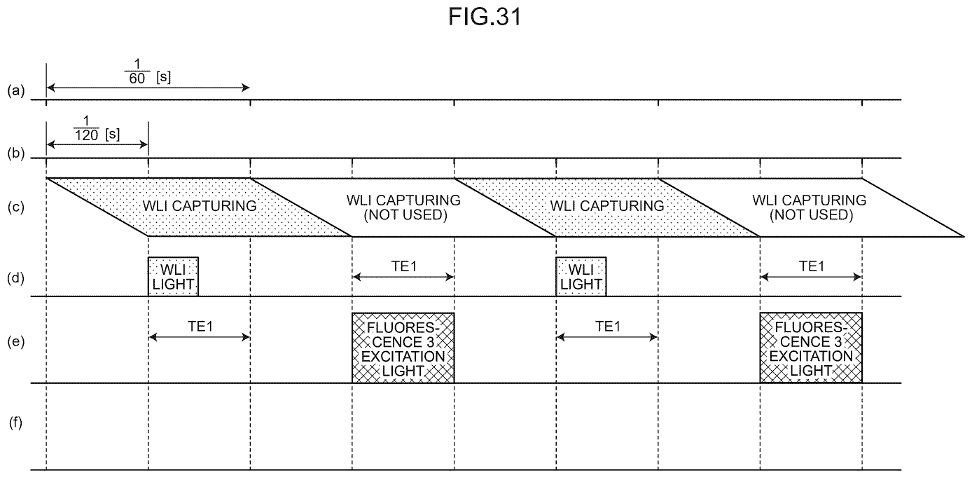

FIG. 31 is a diagram illustrating the operations of the second imaging unit 52 and the light source device 3 in the third mode. Specifically, (a) to (e) of FIG. 31 are diagrams corresponding to (a) to (d) and (f) of FIG. 24, respectively. (f) of FIG. 31 is a diagram corresponding to (d) of FIG. 6.

When set to the third mode, the control unit 94 controls the operations of the second imaging unit 52 and the light source device 3 as illustrated in FIG. 31 as in the case where the control unit 94 is set to the third mode in Modification Example 2 above.

Through the operation of the second imaging unit 52 and the operation of the light source device 3 described above, the normal observation image for left eye is generated.

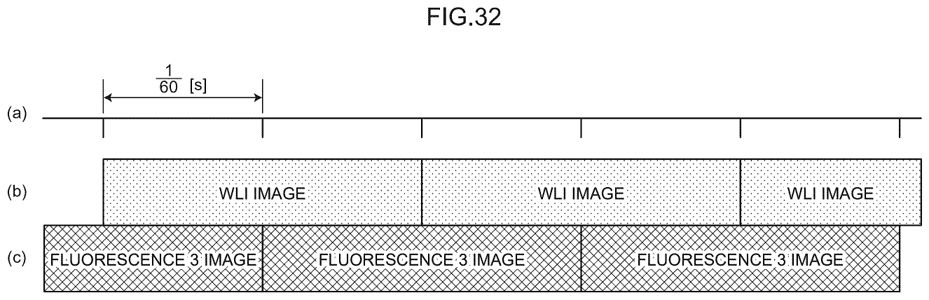

FIG. 32 is a diagram illustrating the updated image of the captured image generated in the third mode. Specifically, (a) to (c) of FIG. 32 are diagrams corresponding to (a) to (c) of FIG. 11, respectively.

Through the operations of the first and second imaging units 51 and 52 and the operation of the light source device 3 described above, each of the normal observation image for right eye, the normal observation image for left eye, and the third fluorescence observation image (fluorescence observation light for right eye) that are generated is updated for every two frames (period of 1/30 [s]) as illustrated in FIG. 32.

The fifth mode is a mode of generating the normal observation image for right eye, the normal observation image for left eye, and the fourth fluorescence observation image.

Hereinafter, operations of the first imaging unit 51 and the light source device 3 in the fifth mode, operations of the second imaging unit 52 and the light source device 3 in the fifth mode, and an updated image of the captured image generated in the fifth mode will be sequentially described.

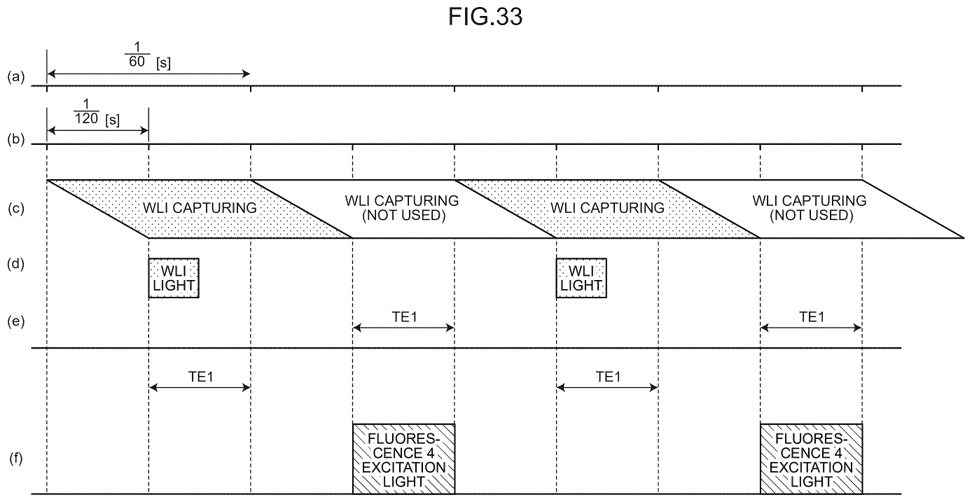

FIG. 33 is a diagram illustrating the operations of the first imaging unit 51 and the light source device 3 in the fifth mode. Specifically, (a) to (f) of FIG. 33 are diagrams corresponding to (a) to (f) of FIG. 30, respectively.

When set to the fifth mode, the control unit 94 executes a capturing control of the first image sensor 513 as illustrated in (c) of FIG. 33 as in the case where the control unit 94 is set to the third mode.

In addition, as illustrated in (d) and (f) of FIG. 33, in an all-line exposure period TE1 for each frame (period of 1/60 [s]) in the first image sensor 513, the control unit 94 alternately repeats the emission of the white light from the first light source 31 and the emission of the fourth fluorescence from the second light source 32. Here, the supply time of the power supplied to the first light source 31 is shorter than the all-line exposure period TE1 ( 1/120 [s]). As a result, the first light source 31 emits the white light for a time of shorter than the all-line exposure period TE1 ( 1/120 [s]). On the other hand, the supply time of the power supplied to the second light source 32 is the all-line exposure period TE1. As a result, the second light source 32 emits the fourth excitation light for a time of the all-line exposure period TE1 ( 1/120 [s]). Therefore, as illustrated in (c) of FIG. 33 the first image sensor 513 captures the white light and the fourth fluorescence in a time division manner for each frame. Here, the normal observation image for right eye that is captured in a period where the fourth fluorescence is emitted is not used. In (f) of FIG. 33, for convenience of description, a text “Fluorescence 4 Excitation Light” representing emission of the excitation light LE4 (fourth excitation light) is illustrated in rectangular regions representing the supply state of power.

Through the operation of the first imaging unit 51 and the operation of the light source device 3 described above, the normal observation image for right eye is generated.

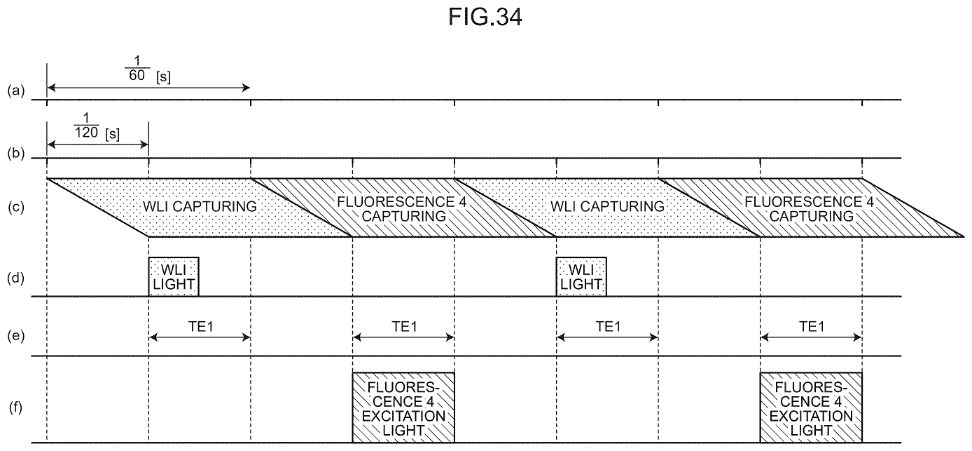

FIG. 34 is a diagram illustrating the operations of the second imaging unit 52 and the light source device 3 in the fifth mode. Specifically, (a) to (f) of FIG. 34 are diagrams corresponding to (a) to (f) of FIG. 31, respectively.

When set to the fifth mode, the control unit 94 controls the operations of the second imaging unit 52 and the light source device 3 as described below.

As illustrated in (c) of FIG. 34, the control unit 94 executes a capturing control of the third image sensor 523 as in the case where the control unit 94 is set to the third mode. Therefore, the third image sensor 523 captures the white light and the fourth fluorescence in a time division manner for each frame. For convenience of description, in (c) of FIG. 34, a text “Fluorescence 4 Capturing” representing the capturing of the fluorescence LF4 (fourth fluorescence) is illustrated in the parallelogram regions.

Through the operation of the second imaging unit 52 and the operation of the light source device 3 described above, the normal observation image for left eye and the fourth fluorescence observation image (fluorescence observation light for left eye) are generated.

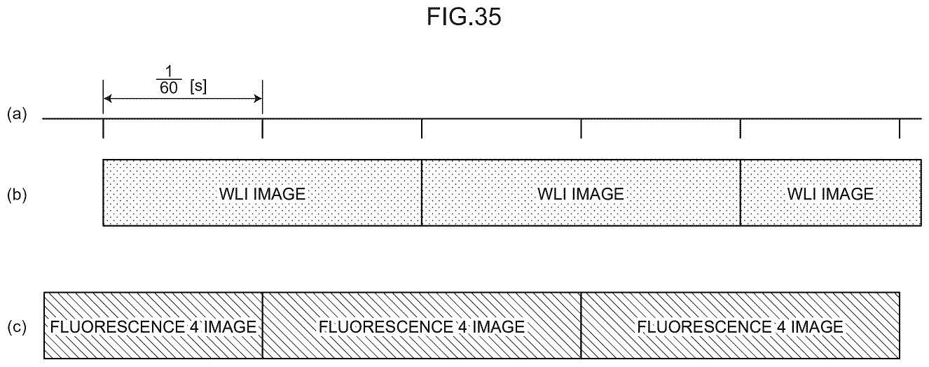

FIG. 35 is a diagram illustrating the updated image of the captured image generated in the fifth mode. Specifically, (a) and (b) of FIG. 35 are diagrams corresponding to (a) and (b) of FIG. 8, respectively. (c) of FIG. 35 is a diagram illustrating the updated image of the fourth fluorescence observation image. For convenience of description, in (c) of FIG. 35, a text “Fluorescence 4 Image” representing the fourth fluorescence observation image is illustrated in rectangular regions representing the fourth fluorescence observation image.

Through the operations of the first and second imaging units 51 and 52 and the operation of the light source device 3 described above, each of the normal observation image for right eye, the normal observation image for left eye, and the fourth fluorescence observation image (fluorescence observation light for left eye) that are generated is updated for every two frames (period of 1/30 [s]) as illustrated in FIG. 35.

Even with the configuration of Modification Example 3 described above, the same effects as in the above-described embodiment are exhibited. In addition, the number of the image sensors is configured to be only two in total. Therefore, miniaturization can be further achieved.

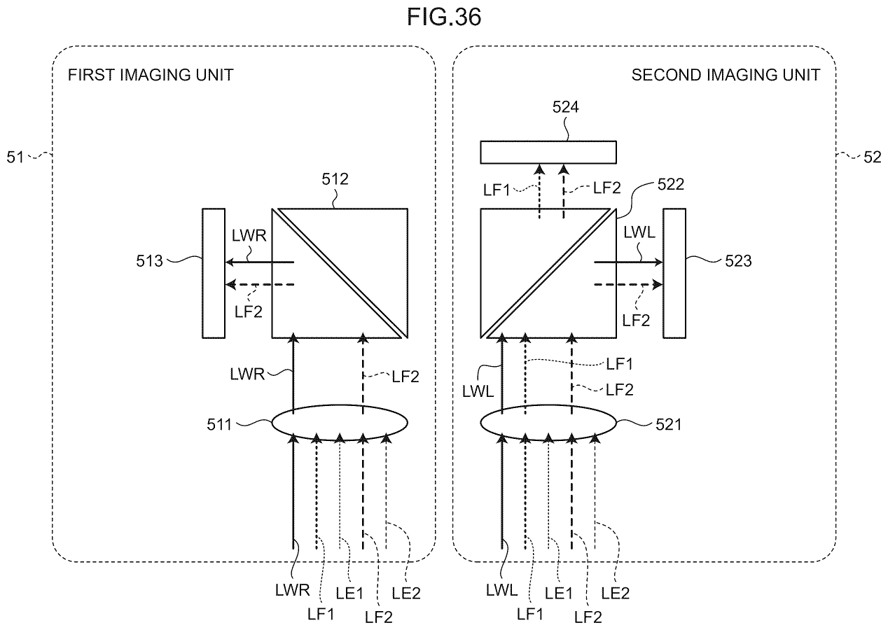

FIG. 36 is a diagram corresponding to FIG. 3 and illustrating a configuration of the first and second imaging units 51 and 52 according to Modification Example 4 of the above-described embodiment.

In the first imaging unit 51 according to Modification Example 4, the second image sensor 514 described in the above-described embodiment is not provided. In addition, in Modification Example 4, the first to fourth optical members 511, 512, 521, and 522 are configured as described below.

The first and second optical members 511 and 512 according to Modification Example 4 have the same functions as the first and second optical members 511 and 512 described above in Modification Example 3.

As illustrated in FIG. 36, the third optical member 521 according to Modification Example 4 has a light cutting function of cutting the excitation light components LE1 and LE2 in the light that is focused in the insertion unit 2 and is incident on the third optical member 521. The light that is focused in the insertion unit 2 and is incident on the third optical member 521 includes the normal observation light for left eye LWL, the excitation light components LE1 and LE2, and the fluorescence components LF1 and LF2.

As illustrated in FIG. 36, the fourth optical member 522 according to Modification Example 4 has a light separating function of separating a part of the fluorescence LF2, the normal observation light for left eye LWL, the fluorescence LF1, and the other fluorescence LF2 that are not cut by the third optical member 521. The fourth optical member 522 allows a part of the fluorescence LF2 and the normal observation light for left eye LWL to travel toward the third image sensor 523, and allows the fluorescence LF1 and the other fluorescence LF2 to travel toward the fourth image sensor 524.

In Modification Example 4, a correspondence between the first, third, and fourth image sensors 513, 523 and 524, the white light, and the first to third fluorescence components is as shown in Table 5 below.

TABLE 5

| | First | Third | Fourth |
|---|---|---|---|
| | image | image | image |
| | sensor | sensor | sensor |
| White light | ⊚ | ⊚ | X |
| First | X | X | ◯ |
| fluorescence | | | |
| Second | X | X | ◯ |
| fluorescence | | | |
| Third | ⊚ | ⊚ | (⊚) |
| fluorescence | | | |

In Table 5, “©” and “(©)” represent being capturable three-dimensionally (3D). That is, a 3D image can be generated by the normal observation image for right eye generated in the first image sensor 513 and the normal observation image for left eye generated in the third image sensor 523. In addition, a 3D image can be generated by the third fluorescence observation image (first stereoscopic fluorescence observation image (fluorescence observation light for right eye (first fluorescence observation light))) generated by the first image sensor 513 and the third fluorescence observation image (second stereoscopic fluorescence observation image (fluorescence observation light for left eye (second fluorescence observation light))) generated by the third image sensor 523. Further, a 3D image can be generated by the third fluorescence observation image (first stereoscopic fluorescence observation image (fluorescence observation light for right eye (first fluorescence observation light))) generated by the first image sensor 513 and the third fluorescence observation image (second stereoscopic fluorescence observation image (fluorescence observation light for left eye (second fluorescence observation light))) generated by the fourth image sensor 524. In addition, “◯” represents being capturable two-dimensionally (in 2D). That is, the 2D first fluorescence observation image can be generated by the fourth image sensor 524. In addition, the 2D second fluorescence observation image can be generated by the fourth image sensor 524. Further, “X” represents not being supported.

Hereinafter, the operations corresponding to the first to fourth modes according to Modification Example 4 will be sequentially described.

Hereinafter, operations of the first imaging unit 51 and the light source device 3 in the third mode, operations of the second imaging unit 52 and the light source device 3 in the third mode, and an updated image of the captured image generated in the third mode will be sequentially described.

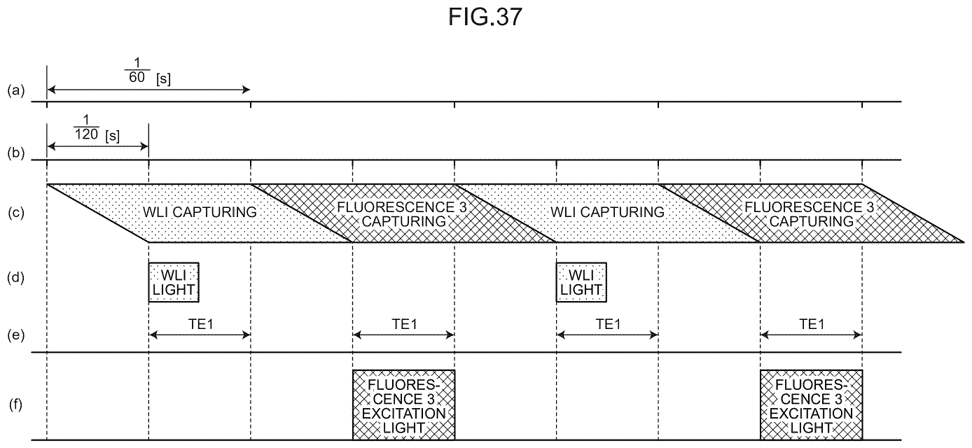

FIG. 37 is a diagram illustrating the operations of the first imaging unit 51 and the light source device 3 in the third mode. Specifically, (a) to (d) and (f) of FIG. 37 are diagrams corresponding to (a) to (e) of FIG. 16, respectively. (e) of FIG. 37 is a diagram corresponding to (f) of FIG. 6.

When set to the third mode, the control unit 94 controls the operations of the first imaging unit 51 and the light source device 3 as illustrated in FIG. 37 as in the case where the control unit 94 is set to the third mode in Modification Example 1 above.

Through the operation of the first imaging unit 51 and the operation of the light source device 3 described above, the normal observation image for right eye and the third fluorescence observation image (first stereoscopic fluorescence observation image (fluorescence observation light for right eye (first fluorescence observation light))) are generated.

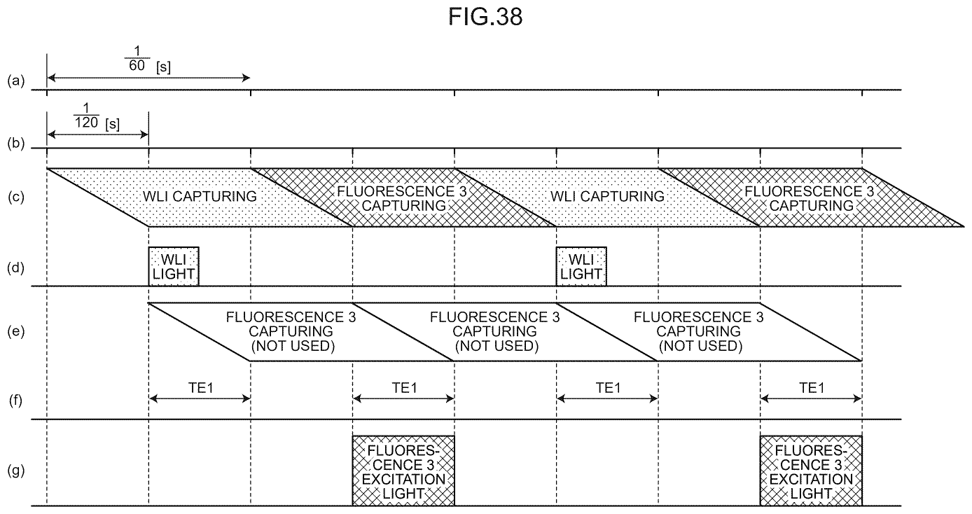

FIG. 38 is a diagram illustrating the operations of the second imaging unit 52 and the light source device 3 in the third mode. Specifically, (a) to (e) and (g) of FIG. 38 are diagrams corresponding to (a) to (f) of FIG. 17, respectively. (f) of FIG. 38 is a diagram corresponding to (f) of FIG. 6.

When set to the third mode, the control unit 94 controls the operations of the second imaging unit 52 and the light source device 3 as illustrated in FIG. 38 as in the case where the control unit 94 is set to the third mode in Modification Example 1 above.

Through the operation of the second imaging unit 52 and the operation of the light source device 3 described above, the normal observation image for left eye and the third fluorescence observation image (second stereoscopic fluorescence observation image (fluorescence observation light for left eye (second fluorescence observation light))) are generated.

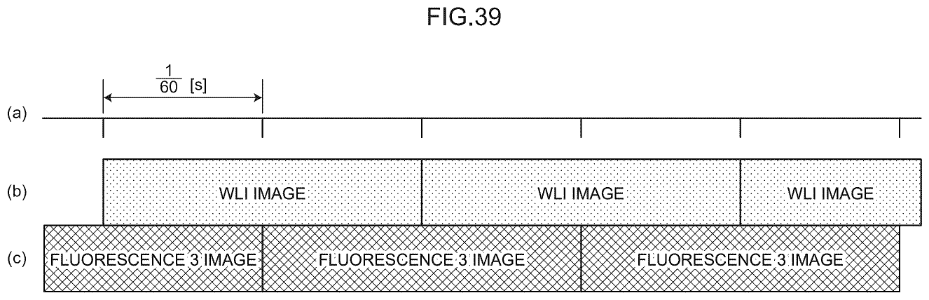

FIG. 39 is a diagram illustrating the updated image of the captured image generated in the third mode. Specifically, (a) to (c) of FIG. 39 are diagrams corresponding to (a) to (c) of FIG. 18, respectively.

Through the operations of the first and second imaging units 51 and 52 and the operation of the light source device 3 described above, each of the normal observation image for right eye, the normal observation image for left eye, and the third fluorescence observation image (fluorescence observation light for right eye, fluorescence observation light for left eye) that are generated is updated for every two frames (period of 1/30 [s]) as illustrated in FIG. 39.

Hereinafter, operations of the first imaging unit 51 and the light source device 3 in the second mode, operations of the second imaging unit 52 and the light source device 3 in the second mode, and an updated image of the captured image generated in the second mode will be sequentially described.

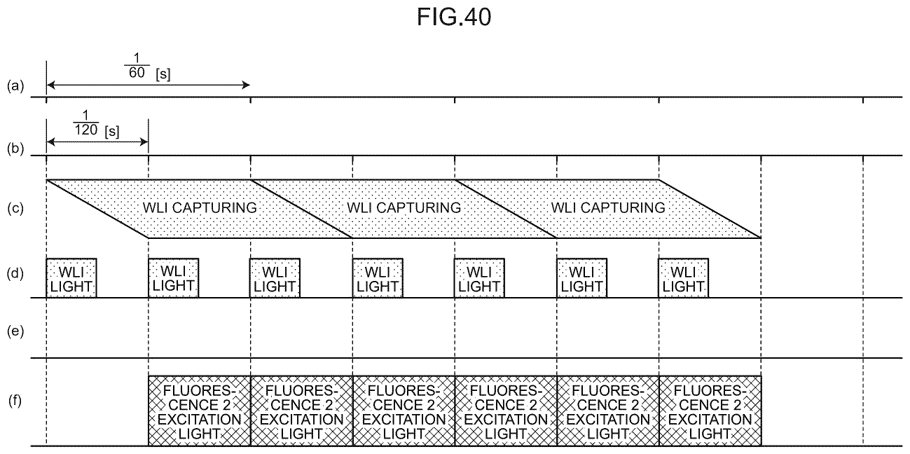

FIG. 40 is a diagram illustrating the operations of the first imaging unit 51 and the light source device 3 in the second mode. Specifically, (a) to (f) of FIG. 40 are diagrams corresponding to (a) to (f) of FIG. 37, respectively.

When set to the second mode, the control unit 94 controls the operations of the first imaging unit 51 and the light source device 3 as described below.

The control unit 94 controls the operations of the first imaging unit 51 and the first light source 31 as illustrated in (c) and (d) of FIG. 40 as in the case where the control unit 94 is set to the second and third modes in the above-described embodiment. In addition, as illustrated in (f) of FIG. 40, the control unit 94 constantly supplies power to the third light source 33. As a result, the third light source 33 is constantly turned on to constantly emit the second excitation light.

Through the operation of the first imaging unit 51 and the operation of the light source device 3 described above, the normal observation image for right eye is generated. The normal observation image for right eye includes the second fluorescence, but the fluorescence intensity of the second fluorescence is weaker than that of the normal observation light for right eye. Therefore, the second fluorescence is embedded in the background (normal observation light for right eye).

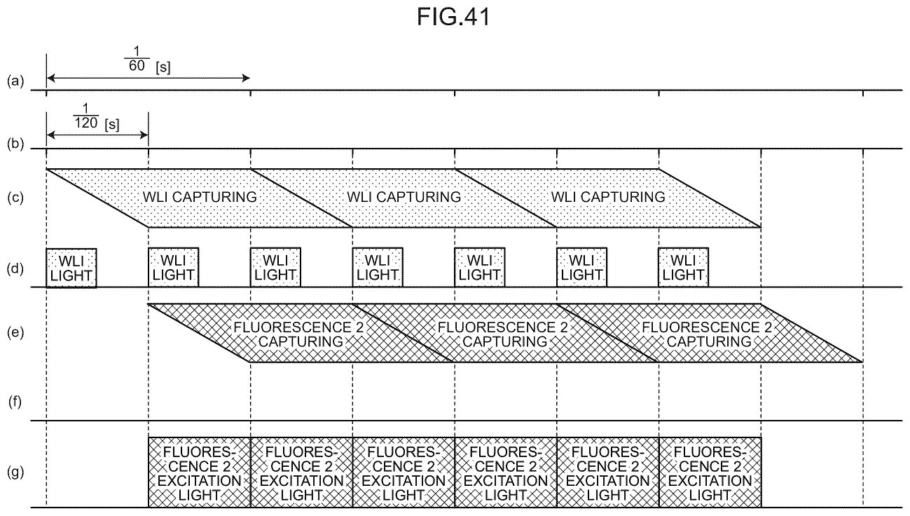

FIG. 41 is a diagram illustrating the operations of the second imaging unit 52 and the light source device 3 in the second mode. Specifically, (a) to (g) of FIG. 41 are diagrams corresponding to (a) to (g) of FIG. 38, respectively. (f) of FIG. 41 is a diagram corresponding to (f) of FIG. 9.

When set to the second mode, the control unit 94 controls the operations of the second imaging unit 52 and the light source device 3 as illustrated in FIG. 41 as in the case where the control unit 94 is set to the second and third modes in the above-described embodiment.

Through the operation of the second imaging unit 52 and the operation of the light source device 3 described above, the normal observation image for left eye and the second fluorescence observation image (fluorescence observation light for left eye) are generated.

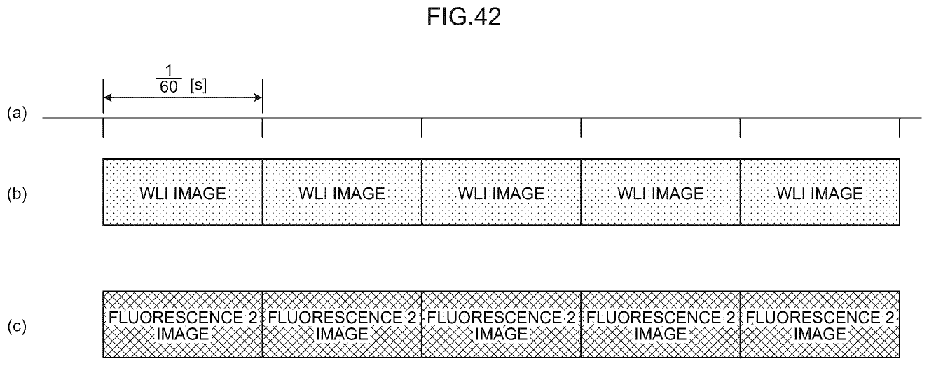

FIG. 42 is a diagram illustrating the updated image of the captured image generated in the second mode. Specifically, (a) to (c) of FIG. 42 are diagrams corresponding to (a) to (c) of FIG. 11, respectively.

Through the operations of the first and second imaging units 51 and 52 and the operation of the light source device 3 described above, each of the normal observation image for right eye, the normal observation image for left eye, and the second fluorescence observation image (fluorescence observation light for left eye) that are generated is updated for each frame (period of 1/60 [s]) as illustrated in FIG. 42.

Hereinafter, operations of the first imaging unit 51 and the light source device 3 in the first mode, operations of the second imaging unit 52 and the light source device 3 in the first mode, and an updated image of the captured image generated in the first mode will be sequentially described.

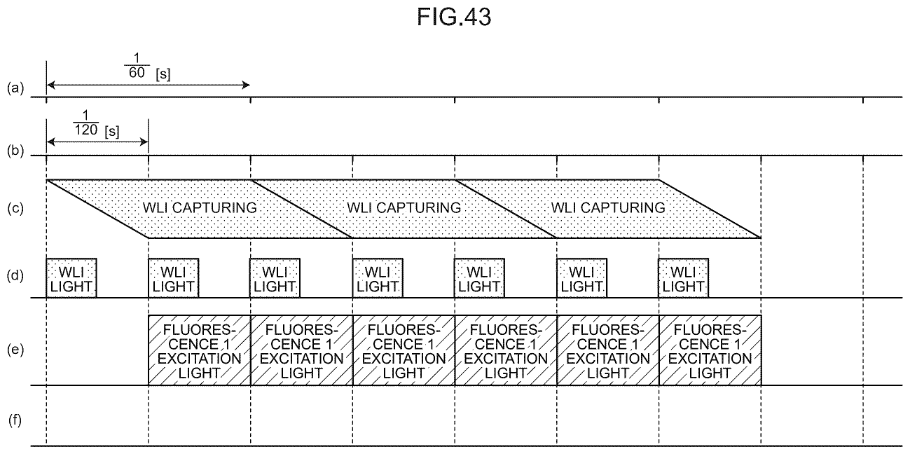

FIG. 43 is a diagram illustrating the operations of the first imaging unit 51 and the light source device 3 in the first mode. Specifically, (a) to (f) of FIG. 43 are diagrams corresponding to (a) to (f) of FIG. 37, respectively.

When set to the first mode, the control unit 94 controls the operations of the first image sensor 513 and the light source device 3 as illustrated in FIG. 43 as in the case where the control unit 94 is set to the first mode in the above-described embodiment.

Through the operation of the first imaging unit 51 and the operation of the light source device 3 described above, the normal observation image for right eye is generated.

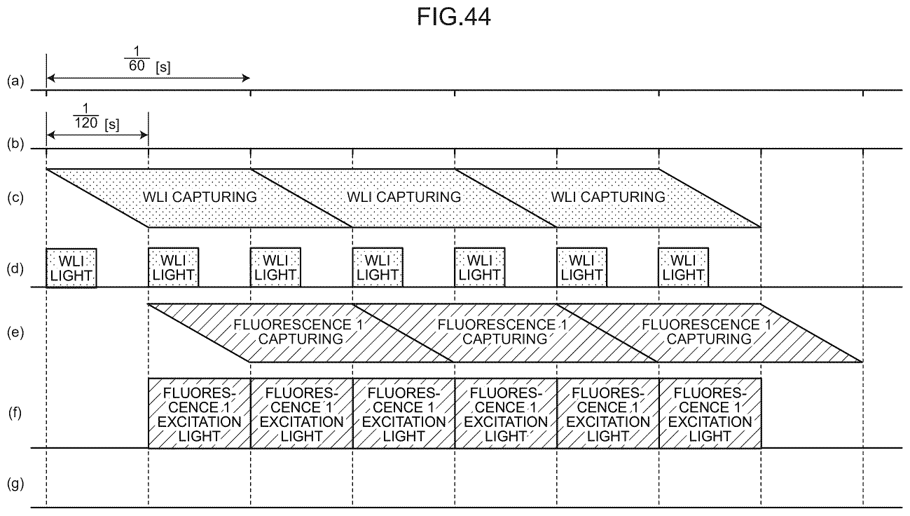

FIG. 44 is a diagram illustrating the operations of the second imaging unit 52 and the light source device 3 in the first mode. Specifically, (a) to (g) of FIG. 44 are diagrams corresponding to (a) to (g) of FIG. 38, respectively.

When set to the first mode, the control unit 94 controls the operations of the second imaging unit 52 and the light source device 3 as described below.

As illustrated in (c) of FIG. 44, the control unit 94 executes a capturing control of the third image sensor 523 as in the case where the control unit 94 is set to the second mode. In addition, as illustrated in (e) of FIG. 44, the control unit 94 executes a capturing control of the fourth image sensor 524 as in the capturing control of the third image sensor 523. At this time, read timings in the respective fields of the third and fourth image sensors 523 and 524 are shifted from each other by 1/120 [s] as illustrated in (c) and (e) of FIG. 44. The read timings of the respective fields may be shifted from each other or may be the same.

Through the operation of the second imaging unit 52 and the operation of the light source device 3 described above, the normal observation image for left eye and the first fluorescence observation image (fluorescence observation light for left eye) are generated.

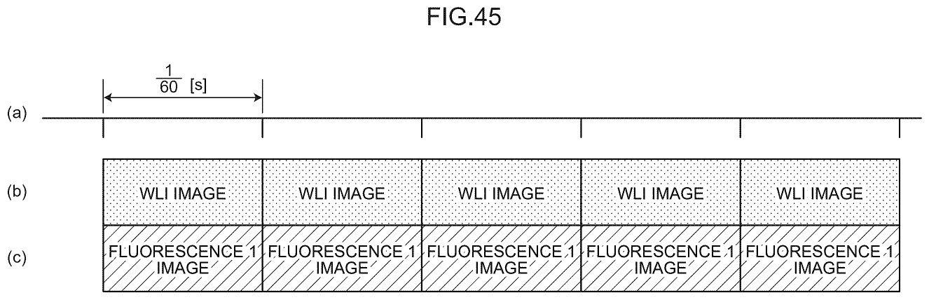

FIG. 45 is a diagram illustrating the updated image of the captured image generated in the first mode. Specifically, (a) to (c) of FIG. 45 are diagrams corresponding to (a) to (c) of FIG. 8, respectively.

Through the operations of the first and second imaging units 51 and 52 and the operation of the light source device 3 described above, each of the normal observation image for right eye, the normal observation image for left eye, and the first fluorescence observation image (fluorescence observation light for left eye) that are generated is updated for each frame (period of 1/60 [s]) as illustrated in FIG. 45.

Hereinafter, operations of the first imaging unit 51 and the light source device 3 in the fourth mode, operations of the second imaging unit 52 and the light source device 3 in the fourth mode, and an updated image of the captured image generated in the fourth mode will be sequentially described.

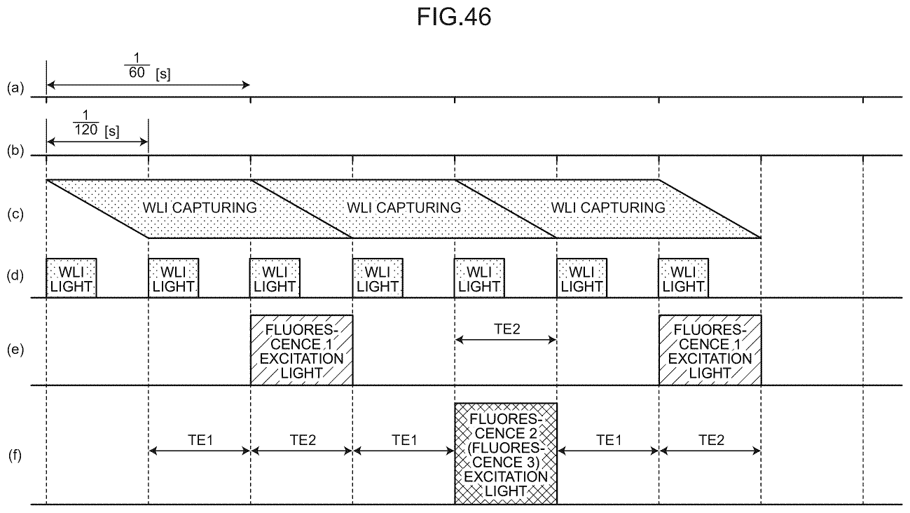

FIG. 46 is a diagram illustrating the operations of the first imaging unit 51 and the light source device 3 in the fourth mode. Specifically, (a) to (f) of FIG. 46 are diagrams corresponding to (a) to (f) of FIG. 37, respectively.

When set to the fourth mode, the control unit 94 controls the operations of the first imaging unit 51 and the light source device 3 as described below.

The control unit 94 controls the operations of the first image sensor 513 and the first light source 31 as illustrated in (c) and (d) of FIG. 46 as in the case where the control unit 94 is set to the second mode.

In addition, as illustrated in (e) and (f) of FIG. 46, in an all-line exposure period TE2 for each frame (period of 1/60 [s]) in the fourth image sensor 524, the control unit 94 alternately repeats the emission of the first excitation light from the second light source 32 and the emission of the second excitation light or the third excitation light from the third light source 33 or the fourth light source 34. Here, the supply time of the power supplied to the second to fourth light sources 32 to 34 is the all-line exposure period TE2 ( 1/120 [s]). As a result, the second light source 32, the third light source 33, or the fourth light source 34 emits the second excitation light or the third excitation light for a time of the all-line exposure period TE2 ( 1/120 [s]).

Through the operation of the first imaging unit 51 and the operation of the light source device 3 described above, the normal observation image for right eye is generated. The normal observation image for right eye includes the second fluorescence or the third fluorescence, but the fluorescence intensity of the second fluorescence or the third fluorescence is weaker than that of the normal observation light for right eye. Therefore, the second fluorescence or the third fluorescence is embedded in the background (normal observation light for right eye).

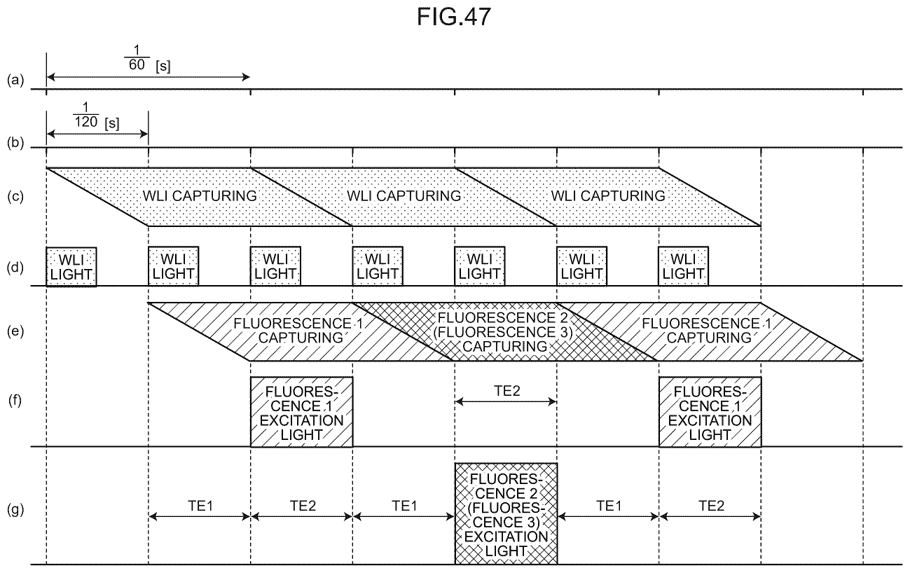

FIG. 47 is a diagram illustrating the operations of the second imaging unit 52 and the light source device 3 in the fourth mode. Specifically, (a) to (g) of FIG. 47 are diagrams corresponding to (a) to (g) of FIG. 38, respectively.

When set to the fourth mode, the control unit 94 controls the operations of the second imaging unit 52 and the light source device 3 as described below.

As illustrated in (c) of FIG. 47, the control unit 94 executes a capturing control of the third image sensor 523 as in the case where the control unit 94 is set to the second mode.

In addition, as illustrated in (e) of FIG. 47, the control unit 94 executes a capturing control of the fourth image sensor 524 as in the case where the control unit 94 is set to the second mode. Therefore, the fourth image sensor 524 captures the first fluorescence and the second fluorescence, or the third fluorescence in a time division manner for each frame.

Through the operations of the second imaging unit 52 and the operation of the light source device 3 described above, the normal observation image for left eye, the first fluorescence observation image (fluorescence observation light for left eye), and the second fluorescence observation image (fluorescence observation light for left eye) or the third fluorescence observation image (fluorescence observation light for left eye) are generated.

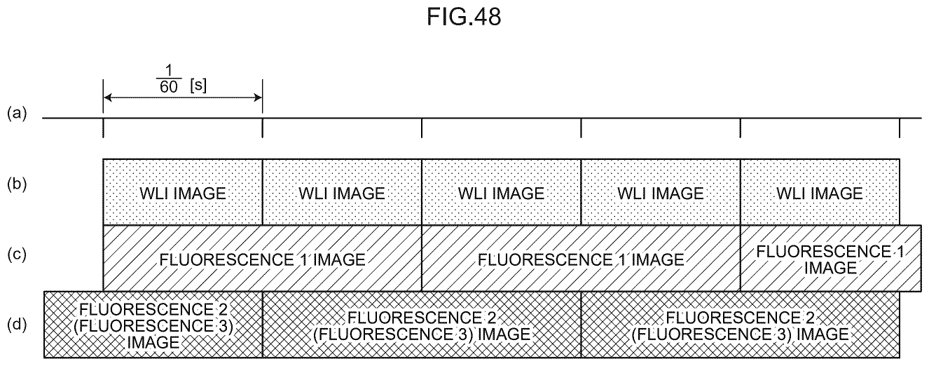

FIG. 48 is a diagram illustrating the updated image of the captured image generated in the fourth mode. Specifically, (a) to (c) of FIG. 48 are diagrams corresponding to (a) to (c) of FIG. 14, respectively.

Through the operations of the first and second imaging units 51 and 52 and the operation of the light source device 3 described above, each of the normal observation image for right eye and the normal observation image for left eye that are generated is updated for each frame (period of 1/60 [s]) as illustrated in FIG. 48. In addition, each of the first fluorescence observation image (fluorescence observation light for left eye) and the second fluorescence observation image (fluorescence observation light for left eye) or the third fluorescence observation image (fluorescence observation light for left eye) is updated for every two frames (period of 1/30 [s]).

Even with the configuration of Modification Example 4 described above, the same effects as in the above-described embodiment are exhibited. In addition, the number of the image sensors is configured to be only three in total. Therefore, miniaturization can be further achieved.

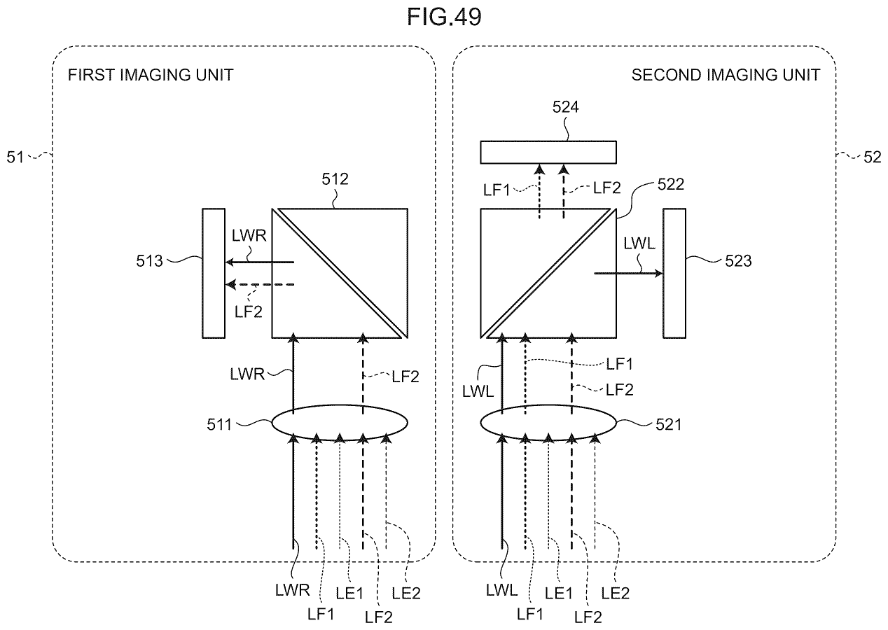

FIG. 49 is a diagram corresponding to FIG. 3 and illustrating a configuration of the first and second imaging units 51 and 52 according to Modification Example 5 of the above-described embodiment.

In the first imaging unit 51 according to Modification Example 5, the second image sensor 514 described in the above-described embodiment is not provided. In addition, in Modification Example 5, the first to fourth optical members 511, 512, 521, and 522 are configured as described below.

The first to third optical members 511, 512, and 521 according to Modification Example 5 have the same functions as the first to third optical members 511, 512, and 521 described above in Modification Example 4.

As illustrated in FIG. 49, the fourth optical member 522 according to Modification Example 5 has a light separating function of separating the normal observation light for left eye LWL and fluorescence components LF1 and LF2 that are not cut by the third optical member 521. The fourth optical member 522 allows the normal observation light for left eye LWL to travel toward the third image sensor 523, and allows the fluorescence components LF1 and LF2 to travel toward the fourth image sensor 524.

In Modification Example 5, a correspondence between the first, third, and fourth image sensors 513, 523 and 524, the white light, and the first to third fluorescence components is as shown in Table 6 below.

TABLE 6

| | First | Third | Fourth |
|---|---|---|---|
| | image | image | image |
| | sensor | sensor | sensor |
| White light | ⊚ | ⊚ | X |
| First | X | X | ◯ |
| fluorescence | | | |
| Second | X | X | ◯ |
| fluorescence | | | |
| Third | ⊚ | X | ⊚ |
| fluorescence | | | |

In Table 6, “©” represents being capturable in 3D. That is, a 3D image can be generated by the normal observation image for right eye generated in the first image sensor 513 and the normal observation image for left eye generated in the third image sensor 523. In addition, a 3D image can be generated by the third fluorescence observation image (first stereoscopic fluorescence observation image (fluorescence observation light for right eye (first fluorescence observation light))) generated by the first image sensor 513 and the third fluorescence observation image (second stereoscopic fluorescence observation image (fluorescence observation light for left eye (second fluorescence observation light))) generated by the fourth image sensor 524. Further, “◯” represents being capturable two-dimensionally (in 2D). That is, the 2D first fluorescence observation image can be generated by the fourth image sensor 524. In addition, the 2D second fluorescence observation image can be generated by the fourth image sensor 524. Further, “X” represents not being supported.

Hereinafter, the operation corresponding to the third mode according to Modification Example 5 will be described. In addition, operations corresponding to the first, second, and fourth modes according to Modification Example 4 are the same as the operations corresponding to the first, second, and fourth modes described above in Modification Example 4.

Hereinafter, operations of the first imaging unit 51 and the light source device 3 in the third mode, operations of the second imaging unit 52 and the light source device 3 in the third mode, and an updated image of the captured image generated in the third mode will be sequentially described.

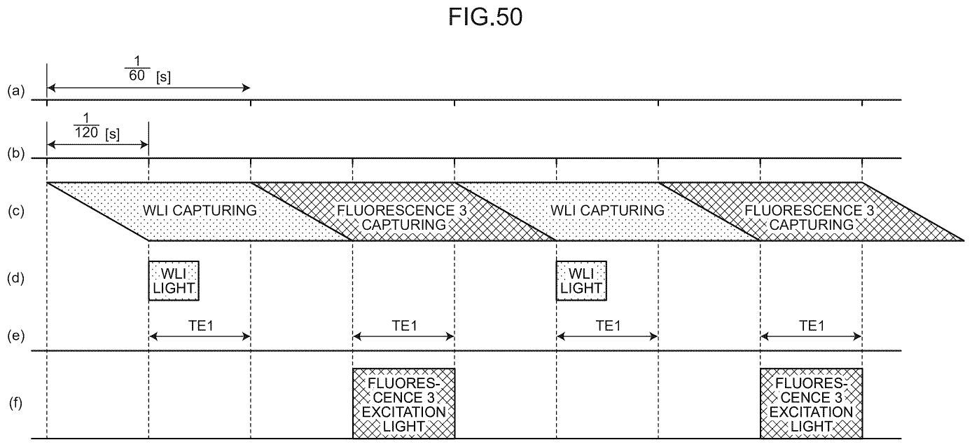

FIG. 50 is a diagram illustrating the operations of the first imaging unit 51 and the light source device 3 in the third mode. Specifically, (a) to (f) of FIG. 50 are diagrams corresponding to (a) to (f) of FIG. 37, respectively.

When set to the third mode, the control unit 94 controls the operations of the first imaging unit 51 and the light source device 3 as illustrated in FIG. 50 as in the case where the control unit 94 is set to the third mode in Modification Example 4 above.

Through the operation of the first imaging unit 51 and the operation of the light source device 3 described above, the normal observation image for right eye and the third fluorescence observation image (first stereoscopic fluorescence observation image (fluorescence observation light for right eye (first fluorescence observation light))) are generated.

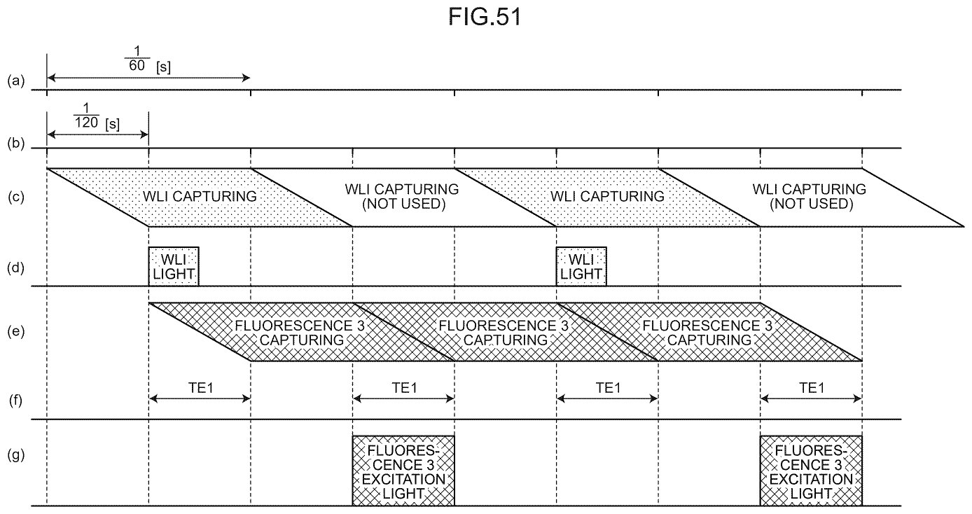

FIG. 51 is a diagram illustrating the operations of the second imaging unit 52 and the light source device 3 in the third mode. Specifically, (a) to (g) of FIG. 51 are diagrams corresponding to (g) of FIG. 38, respectively.

When set to the third mode, the control unit 94 controls the operations of the second imaging unit 52 and the light source device 3 as illustrated in FIG. 51 as in the case where the control unit 94 is set to the third mode in Modification Example 4 above. Here, as illustrated in (c) and (g) of FIG. 51, the normal observation image for left eye that is generated by the third image sensor 523 in a period where the third excitation light is emitted is not used. In addition, as illustrated in (e) of FIG. 51, the third fluorescence observation image that is generated by the fourth image sensor 524 is used.

Through the operation of the second imaging unit 52 and the operation of the light source device 3 described above, the normal observation image for left eye and the third fluorescence observation image (second stereoscopic fluorescence observation image (fluorescence observation light for left eye (second fluorescence observation light))) are generated.

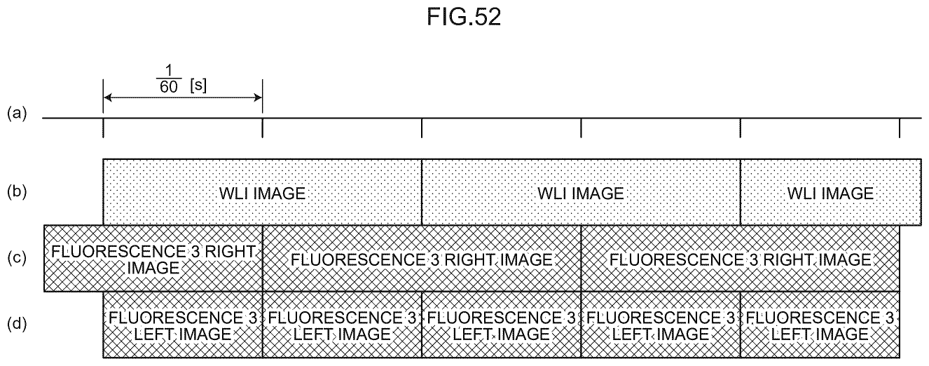

FIG. 52 is a diagram illustrating the updated image of the captured image generated in the third mode. Specifically, (a) to (d) of FIG. 52 are diagrams corresponding to (a) to (d) of FIG. 25, respectively.

Through the operations of the first and second imaging units 51 and 52 and the operation of the light source device 3 described above, each of the normal observation image for right eye, the normal observation image for left eye, and the third fluorescence observation image (first stereoscopic fluorescence observation image (fluorescence observation light for right eye (first fluorescence observation light))) that are generated is updated for every two frames (period of 1/30 [s]) as illustrated in FIG. 52. In addition, the third fluorescence observation image (second stereoscopic fluorescence observation image (fluorescence observation light for left eye (second fluorescence observation light))) is updated for each frame (period of 1/60 [s]).

Even with the configuration of Modification Example 5 described above, the same effects as in the above-described embodiment are exhibited. In addition, the number of the image sensors is configured to be only three in total. Therefore, miniaturization can be further achieved.

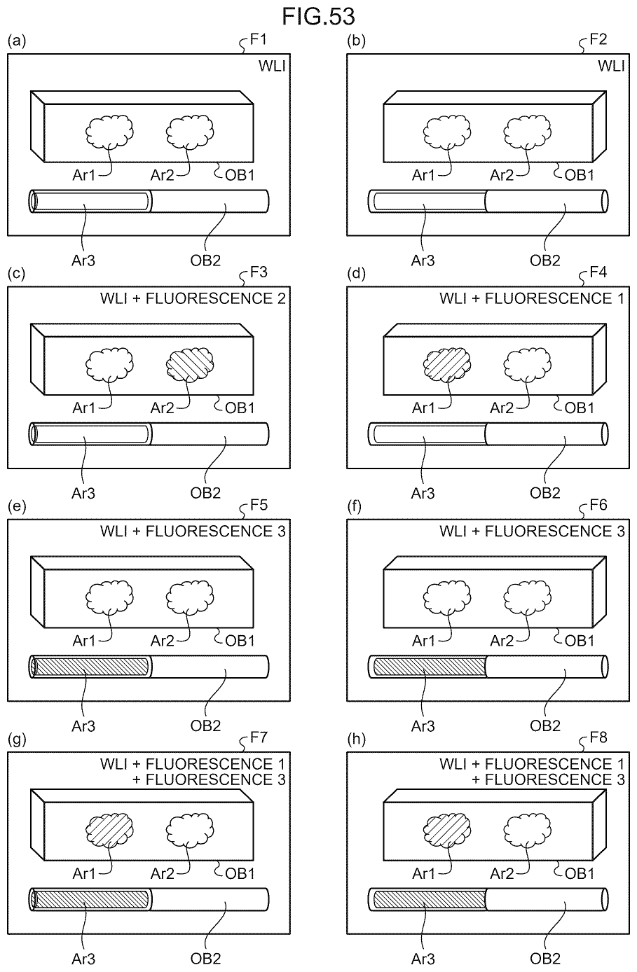

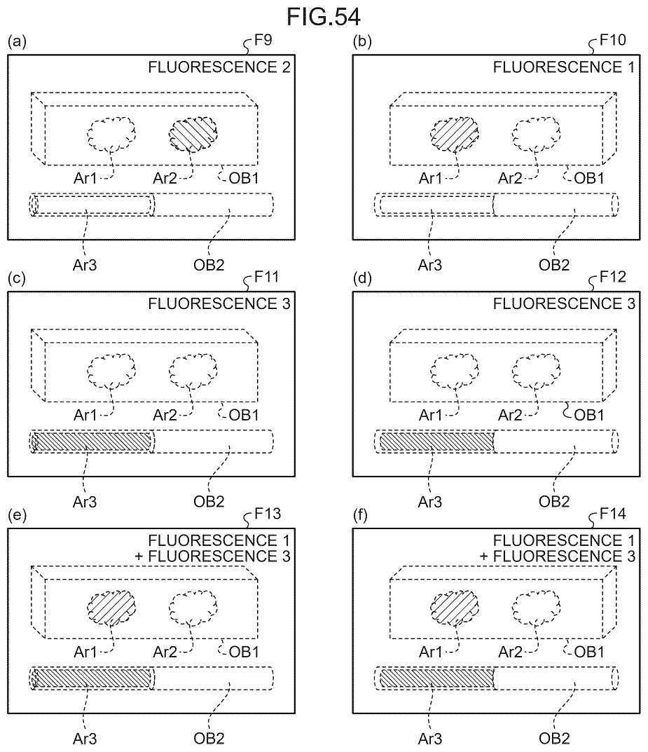

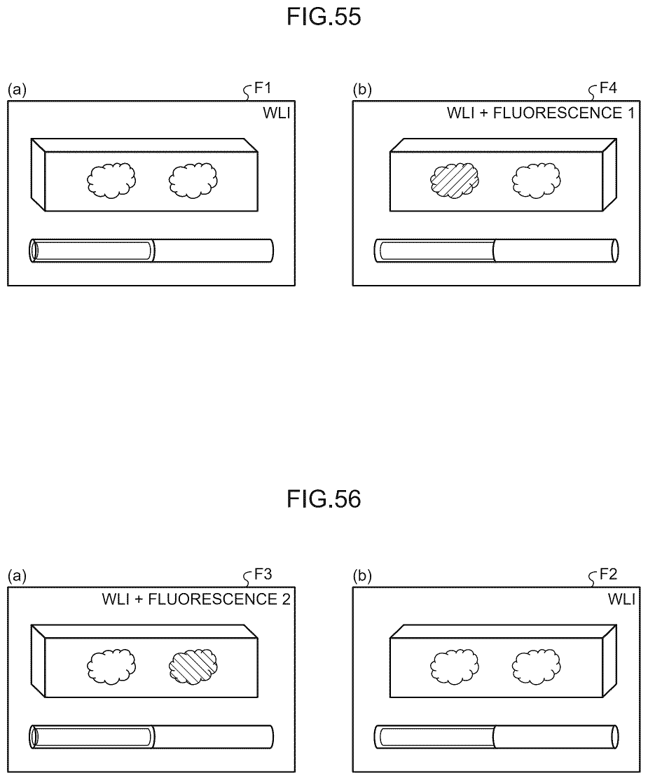

FIGS. 53 to 81 are diagrams illustrating Modification Example 6 of the embodiment. Specifically, FIGS. 53 and 54 are diagrams illustrating examples of the captured images generated in the configurations of the above-described embodiment and Modification Examples 1 to 5 above. FIGS. 55 to 81 are diagrams illustrating examples of a video signal for left eye ((a) of FIG. 55 to (a) of FIG. 81) and a video signal for right eye ((b) of FIG. 55 to (b) of FIG. 81) that are displayed by the display device 7.

In FIGS. 53 and 54, “OB1” is a first observation target representing an organ such as liver. “OB2” is a second observation target representing a blood vessel. Each of “Ar1” to “Ar3” represents a region where fluorescence is emitted by irradiation of excitation light.

In the configurations of the above-described embodiment and Modification Examples 1 to 5, captured images F1 to F14 illustrated in FIGS. 53 and 54 are generated.

The captured image F1 illustrated in (a) of FIG. 53 is the normal observation image for left eye that is generated by the third image sensor 523. In (a) of FIG. 53, for convenience of description, a text “WLI” representing the normal observation image is illustrated in the upper right of the captured image F1.

The captured image F2 illustrated in (b) of FIG. 53 is a normal observation image for right eye that is generated by the first image sensor 513. In (b) of FIG. 53, for convenience of description, a text “WLI” representing the normal observation image is illustrated in the upper right of the captured image F2.

The captured image F3 illustrated in (c) of FIG. 53 is a superimposed image where the second fluorescence observation image generated by the fourth image sensor 524 is superimposed on the normal observation image for left eye generated by the third image sensor 523. This superimposed image (the same also applies to superimposed images described below) is generated by well-known alpha blending or additive blending. For example, the region Ar2 representing the second fluorescence in the second fluorescence observation image is displayed in green or the like. In (c) of FIG. 53, for convenience of description, a text “WLI+Fluorescence 2” representing a superimposed image where the second fluorescence observation image is superimposed on the normal observation image is illustrated in the upper right of the captured image F3.

The captured image F4 illustrated in (d) of FIG. 53 is a superimposed image where the first fluorescence observation image generated by the second image sensor 514 is superimposed on the normal observation image for right eye generated by the first image sensor. For example, the region Ar1 representing the first fluorescence in the first fluorescence observation image is displayed in blue or the like. In (d) of FIG. 53, for convenience of description, a text “WLI+Fluorescence 1” representing a superimposed image where the first fluorescence observation image is superimposed on the normal observation image is illustrated in the upper right of the captured image F4.

The captured image F5 illustrated in (e) of FIG. 53 is a superimposed image where the third fluorescence observation image generated by the fourth image sensor 524 is superimposed on the normal observation image for left eye generated by the third image sensor 523. For example, the region Ar3 representing the third fluorescence in the third fluorescence observation image is displayed in green or the like. In (e) of FIG. 53, for convenience of description, a text “WLI+Fluorescence 3” representing a superimposed image where the third fluorescence observation image is superimposed on the normal observation image is illustrated in the upper right of the captured image F5.

The captured image F6 illustrated in (f) of FIG. 53 is a superimposed image where the third fluorescence observation image generated by the second image sensor 514 is superimposed on the normal observation image for right eye generated by the first image sensor 513. For example, the region Ar3 representing the third fluorescence in the third fluorescence observation image is displayed in green or the like. In (f) of FIG. 53, for convenience of description, a text “WLI+Fluorescence 3” representing a superimposed image where the third fluorescence observation image is superimposed on the normal observation image is illustrated in the upper right of the captured image F6.

The captured image F7 illustrated in (g) of FIG. 53 is a superimposed image where the third fluorescence observation image generated by the third image sensor 523 and the first fluorescence observation image generated by the fourth image sensor 524 are superimposed on the normal observation image for left eye generated by the third image sensor 523. For example, the region Ar3 representing the third fluorescence in the third fluorescence observation image is displayed in green or the like, and the region Ar1 representing the first fluorescence in the first fluorescence observation image is displayed in blue or the like. In (g) of FIG. 53, for convenience of description, a text “WLI+Fluorescence 1+Fluorescence 3” representing a superimposed image where the first and third fluorescence observation images are superimposed on the normal observation image is illustrated in the upper right of the captured image F7.

The captured image F8 illustrated in (h) of FIG. 53 is a superimposed image where the third fluorescence observation image generated by the first image sensor 513 and the first fluorescence observation image generated by the second image sensor 514 are superimposed on the normal observation image for right eye generated by the first image sensor 513. For example, the region Ar3 representing the third fluorescence in the third fluorescence observation image is displayed in green or the like, and the region Ar1 representing the first fluorescence in the first fluorescence observation image is displayed in blue or the like. In (h) of FIG. 53, for convenience of description, a text “WLI+Fluorescence 1+Fluorescence 3” representing a superimposed image where the first and third fluorescence observation images are superimposed on the normal observation image is illustrated in the upper right of the captured image F8.

The captured image F9 illustrated in (a) of FIG. 54 is the second fluorescence observation image that is generated by the fourth image sensor 524. The second and fourth image sensors 524 are configured to execute capturing in monochrome. Therefore, in (a) of FIG. 54 (the same applies to (b) to (f) of FIG. 54), the first and second observation targets OB1 and OB2 that are not displayed are represented by broken lines. In addition, in (a) of FIG. 54, the region Ar2 where the fluorescence intensity of the second fluorescence is strong is represented by diagonal lines. In (a) of FIG. 54, for convenience of description, a text “Fluorescence 2” representing the second fluorescence observation image is illustrated in the upper right of the captured image F9.

The captured image F10 illustrated in (b) of FIG. 54 is the first fluorescence observation image that is generated by the second image sensor 514. In (b) of FIG. 54, the region Ar1 where the fluorescence intensity of the first fluorescence is strong is represented by diagonal lines. In addition, in (b) of FIG. 54, for convenience of description, a text “Fluorescence 1” representing the first fluorescence observation image is illustrated in the upper right of the captured image F10.

The captured image F11 illustrated in (c) of FIG. 54 is the third fluorescence observation image that is generated by the fourth image sensor 524. In (c) of FIG. 54, the region Ar3 where the fluorescence intensity of the third fluorescence is strong is represented by diagonal lines. In addition, in (c) of FIG. 54, for convenience of description, a text “Fluorescence 3” representing the third fluorescence observation image is illustrated in the upper right of the captured image F11.

The captured image F12 illustrated in (d) of FIG. 54 is the third fluorescence observation image that is generated by the first image sensor 513. In (d) of FIG. 54, the region Ar3 where the fluorescence intensity of the third fluorescence is strong is represented by diagonal lines. In addition, in (d) of FIG. 54, for convenience of description, a text “Fluorescence 3” representing the third fluorescence observation image is illustrated in the upper right of the captured image F12.

The captured image F13 illustrated in (e) of FIG. 54 is a superimposed image where the third fluorescence observation image generated by the third image sensor 523 and the first fluorescence observation image generated by the fourth image sensor 524 are superimposed. For example, the region Ar1 representing the first fluorescence in the first fluorescence observation image is displayed in blue or the like, and the region Ar3 representing the third fluorescence in the third fluorescence observation image is displayed in green or the like. In (e) of FIG. 54, for convenience of description, a text “Fluorescence 1+Fluorescence 3” representing a superimposed image where the first and third fluorescence observation images are superimposed is illustrated in the upper right of the captured image F13.

The captured image F14 illustrated in (f) of FIG. 54 is a superimposed image where the third fluorescence observation image generated by the first image sensor 513 and the first fluorescence observation image generated by the second image sensor 514 are superimposed. For example, the region Ar1 representing the first fluorescence in the first fluorescence observation image is displayed in blue or the like, and the region Ar3 representing the third fluorescence in the third fluorescence observation image is displayed in green or the like. In (f) of FIG. 54, for convenience of description, a text “Fluorescence 1+Fluorescence 3” representing a superimposed image where the first and third fluorescence observation images are superimposed is illustrated in the upper right of the captured image F14.

The display control unit 933 generates a display image that enables stereoscopic observation using the video signal for left eye and the video signal for right eye described below. Examples of a system for the stereoscopic observation include a top-and-bottom system, a side-by-side system, and a line-by-line system.

In the example of FIG. 55, the display control unit 933 displays the captured image F1 as the video signal for left eye ((a) of FIG. 55) and displays the captured image F4 as the video signal for right eye ((b) of FIG. 55). That is, the normal observation image corresponding to the white light is displayed in 3D, and the region representing the first fluorescence is displayed in 2D.

In the example of FIG. 56, the display control unit 933 displays the captured image F3 as the video signal for left eye ((a) of FIG. 56) and displays the captured image F2 as the video signal for right eye ((b) of FIG. 56). That is, the normal observation image corresponding to the white light is displayed in 3D, and the region representing the second fluorescence is displayed in 2D.

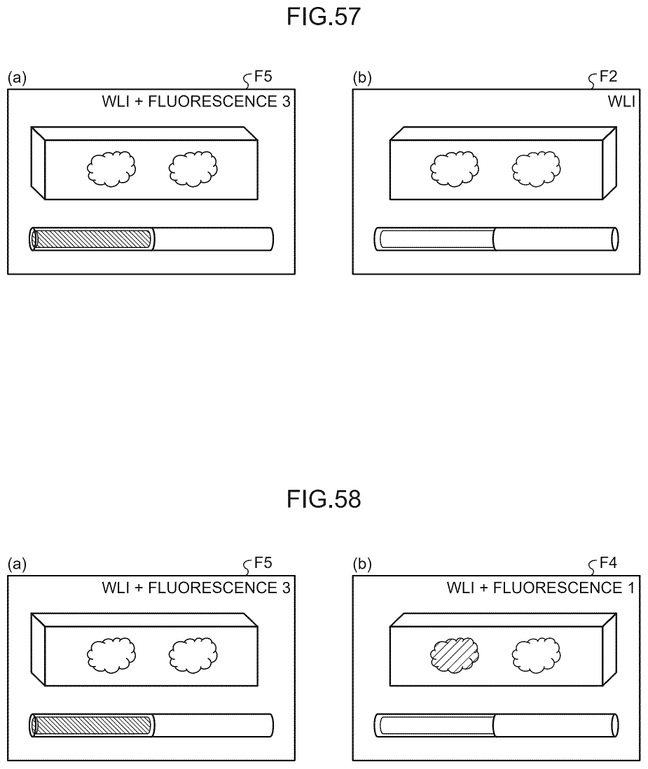

In the example of FIG. 57, the display control unit 933 displays the captured image F5 as the video signal for left eye ((a) of FIG. 57) and displays the captured image F2 as the video signal for right eye ((b) of FIG. 57). That is, the normal observation image corresponding to the white light is displayed in 3D, and the region where the fluorescence intensity of the third fluorescence is strong is displayed in 2D.

In the example of FIG. 58, the display control unit 933 displays the captured image F5 as the video signal for left eye ((a) of FIG. 58) and displays the captured image F4 as the video signal for right eye ((b) of FIG. 58). That is, the normal observation image corresponding to the white light is displayed in 3D, and the regions where the fluorescence intensities of the first and third fluorescence components are strong are displayed in 2D.

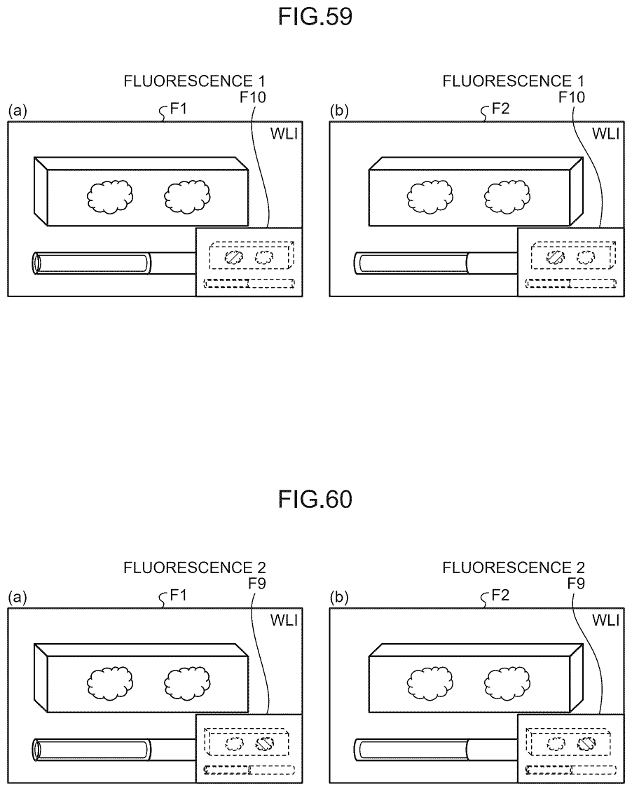

A display method of FIGS. 59 to 81 described below is a display method for obtaining a display image that can be seen more naturally, as compared to the display method in which the normal observation image corresponding to the white light is displayed in 3D and the region representing the fluorescence is displayed in 2D.

In the example of FIG. 59, the display control unit 933 displays an image where a first screen (parent screen) is the captured image F1 and a second screen (child screen) is the captured image F10 as the video signal for left eye ((a) of FIG. 59), and displays an image where the first screen (parent screen) is the captured image F2 and the second screen (child screen) is the captured image F10 as the video signal for right eye ((b) of FIG. 59). That is, the first screen is displayed in 3D, and the second screen is displayed in 2D (PinP display).

In the example of FIG. 60, the display control unit 933 displays an image where the first screen (parent screen) is the captured image F1 and the second screen (child screen) is the captured image F9 as the video signal for left eye ((a) of FIG. 60), and displays an image where the first screen (parent screen) is the captured image F2 and the second screen (child screen) is the captured image F9 as the video signal for right eye ((b) of FIG. 60). That is, the first screen is displayed in 3D, and the second screen is displayed in 2D (PinP display).

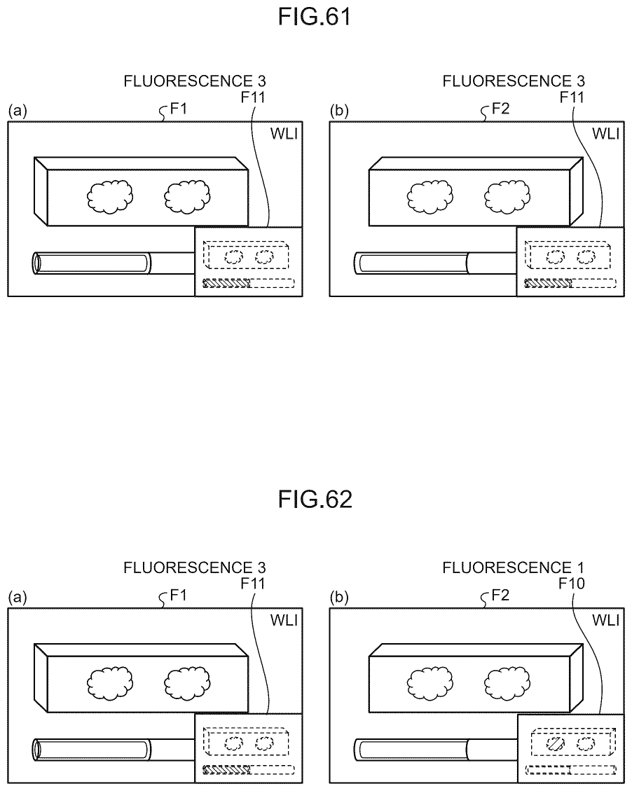

In the example of FIG. 61, the display control unit 933 displays an image where the first screen (parent screen) is the captured image F1 and the second screen (child screen) is the captured image F11 as the video signal for left eye ((a) of FIG. 61), and displays an image where the first screen (parent screen) is the captured image F2 and the second screen (child screen) is the captured image F11 as the video signal for right eye ((b) of FIG. 61). That is, the first screen is displayed in 3D, and the second screen is displayed in 2D (PinP display).

In the example of FIG. 62, the display control unit 933 displays an image where the first screen (parent screen) is the captured image F1 and the second screen (child screen) is the captured image F11 as the video signal for left eye ((a) of FIG. 62), and displays an image where the first screen (parent screen) is the captured image F2 and the second screen (child screen) is the captured image F10 as the video signal for right eye ((b) of FIG. 62). That is, the first screen is displayed in 3D, and the second screen is displayed in 2D (PinP display).

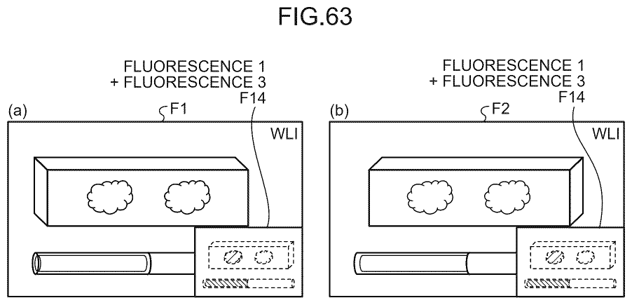

In the example of FIG. 63, the display control unit 933 displays an image where the first screen (parent screen) is the captured image F1 and the second screen (child screen) is the captured image F14 as the video signal for left eye ((a) of FIG. 63), and displays an image where the first screen (parent screen) is the captured image F2 and the second screen (child screen) is the captured image F14 as the video signal for right eye ((b) of FIG. 63). That is, the first screen is displayed in 3D, and the second screen is displayed in 2D (PinP display).

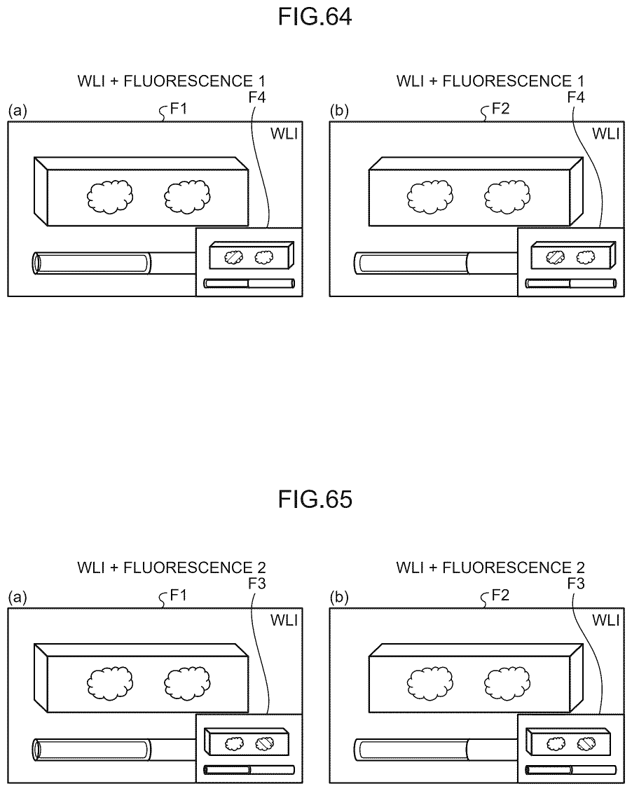

In the example of FIG. 64, the display control unit 933 displays an image where the first screen (parent screen) is the captured image F1 and the second screen (child screen) is the captured image F4 as the video signal for left eye ((a) of FIG. 64), and displays an image where the first screen (parent screen) is the captured image F2 and the second screen (child screen) is the captured image F4 as the video signal for right eye ((b) of FIG. 62). That is, the first screen is displayed in 3D, and the second screen is displayed in 2D (PinP display).

In the example of FIG. 65, the display control unit 933 displays an image where the first screen (parent screen) is the captured image F1 and the second screen (child screen) is the captured image F3 as the video signal for left eye ((a) of FIG. 65), and displays an image where the first screen (parent screen) is the captured image F2 and the second screen (child screen) is the captured image F3 as the video signal for right eye ((b) of FIG. 65). That is, the first screen is displayed in 3D, and the second screen is displayed in 2D (PinP display).

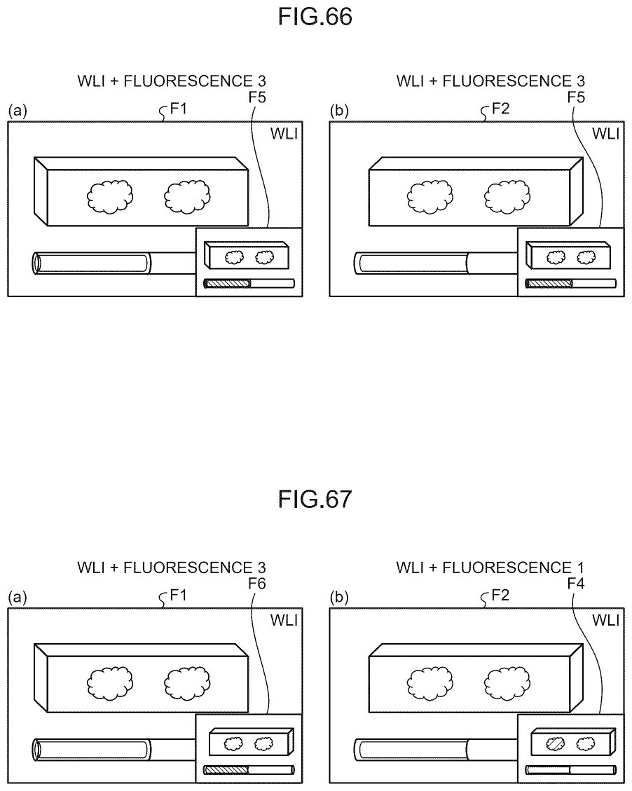

In the example of FIG. 66, the display control unit 933 displays an image where the first screen (parent screen) is the captured image F1 and the second screen (child screen) is the captured image F5 as the video signal for left eye ((a) of FIG. 66), and displays an image where the first screen (parent screen) is the captured image F2 and the second screen (child screen) is the captured image F5 as the video signal for right eye ((b) of FIG. 66). That is, the first screen is displayed in 3D, and the second screen is displayed in 2D (PinP display).

In the example of FIG. 67, the display control unit 933 displays an image where the first screen (parent screen) is the captured image F1 and the second screen (child screen) is the captured image F6 as the video signal for left eye ((a) of FIG. 67), and displays an image where the first screen (parent screen) is the captured image F2 and the second screen (child screen) is the captured image F4 as the video signal for right eye ((b) of FIG. 67). That is, the first screen is displayed in 3D, and the second screen is displayed in 2D (PinP display).

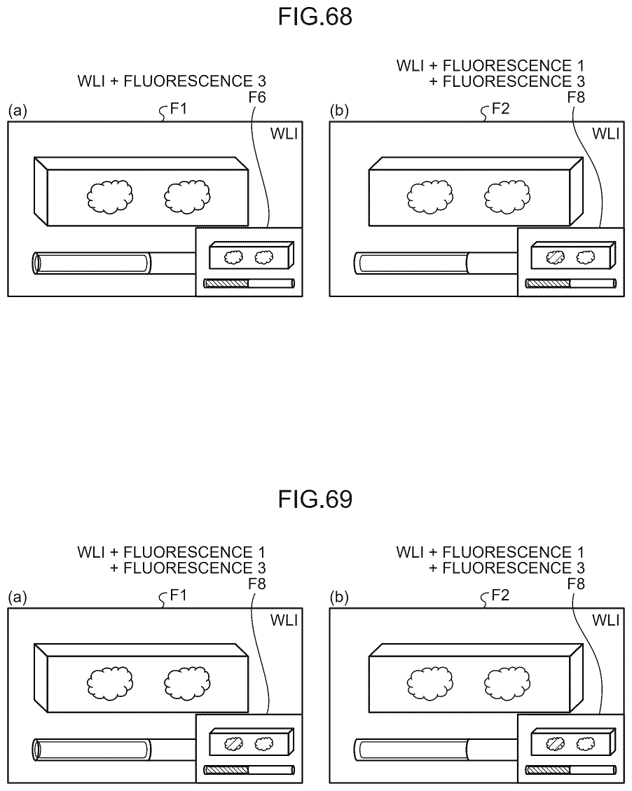

In the example of FIG. 68, the display control unit 933 displays an image where the first screen (parent screen) is the captured image F1 and the second screen (child screen) is the captured image F6 as the video signal for left eye ((a) of FIG. 68), and displays an image where the first screen (parent screen) is the captured image F2 and the second screen (child screen) is the captured image F8 as the video signal for right eye ((b) of FIG. 68). That is, the first screen is displayed in 3D, and the second screen is displayed in 2D (PinP display).

In the example of FIG. 69, the display control unit 933 displays an image where the first screen (parent screen) is the captured image F1 and the second screen (child screen) is the captured image F8 as the video signal for left eye ((a) of FIG. 69), and displays an image where the first screen (parent screen) is the captured image F2 and the second screen (child screen) is the captured image F8 as the video signal for right eye ((b) of FIG. 69). That is, the first screen is displayed in 3D, and the second screen is displayed in 2D (PinP display).

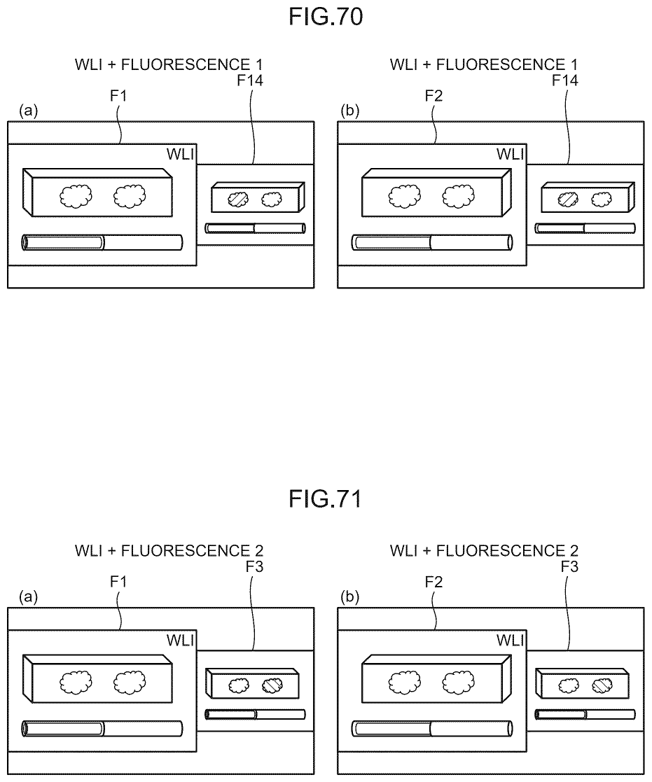

In the example of FIG. 70, the display control unit 933 displays an image where the first screen is the captured image F1 and the second screen is the captured image F14 displayed parallel to the first screen as the video signal for left eye ((a) of FIG. 70), and displays an image where the first screen is the captured image F2 and the second screen is the captured image F14 as the video signal for right eye ((b) of FIG. 70). That is, the first screen is displayed in 3D, and the second screen is displayed in 2D (PoutP display).

In the example of FIG. 71, the display control unit 933 displays an image where the first screen is the captured image F1 and the second screen is the captured image F3 as the video signal for left eye ((a) of FIG. 71), and displays an image where the first screen is the captured image F2 and the second screen is the captured image F3 as the video signal for right eye ((b) of FIG. 71). That is, the first screen is displayed in 3D, and the second screen is displayed in 2D (PoutP display).

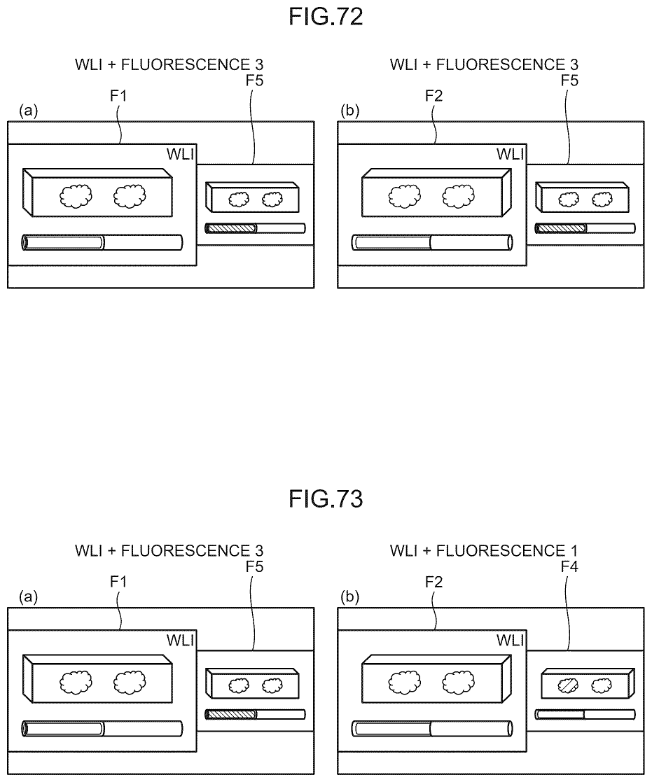

In the example of FIG. 72, the display control unit 933 displays an image where the first screen is the captured image F1 and the second screen is the captured image F5 as the video signal for left eye ((a) of FIG. 72), and displays an image where the first screen is the captured image F2 and the second screen is the captured image F5 as the video signal for right eye ((b) of FIG. 72). That is, the first screen is displayed in 3D, and the second screen is displayed in 2D (PoutP display).

In the example of FIG. 73, the display control unit 933 displays an image where the first screen is the captured image F1 and the second screen is the captured image F5 as the video signal for left eye ((a) of FIG. 73), and displays an image where the first screen is the captured image F2 and the second screen is the captured image F4 as the video signal for right eye ((b) of FIG. 73). That is, the normal light images on the first screen and the second screen are displayed in 3D, and the region representing the first and third fluorescence components on the second screen is displayed in 2D (PoutP display).

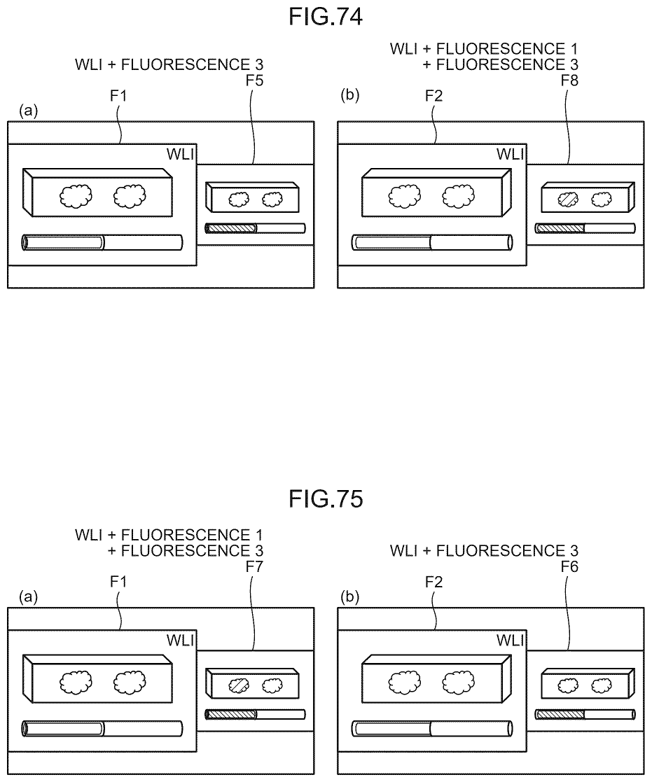

In the example of FIG. 74, the display control unit 933 displays an image where the first screen is the captured image F1 and the second screen is the captured image F5 as the video signal for left eye ((a) of FIG. 74), and displays an image where the first screen is the captured image F2 and the second screen is the captured image F8 as the video signal for right eye ((b) of FIG. 74). That is, the normal light image and the region representing the third fluorescence on the first screen and the second screen are displayed in 3D, and the region representing the first fluorescence on the second screen is displayed in 2D (PoutP display).

In the example of FIG. 75, the display control unit 933 displays an image where the first screen is the captured image F1 and the second screen is the captured image F7 as the video signal for left eye ((a) of FIG. 75), and displays an image where the first screen is the captured image F2 and the second screen is the captured image F6 as the video signal for right eye ((b) of FIG. 75). That is, the normal light image and the region representing the third fluorescence on the first screen and the second screen are displayed in 3D, and the region representing the first fluorescence on the second screen is displayed in 2D (PoutP display).

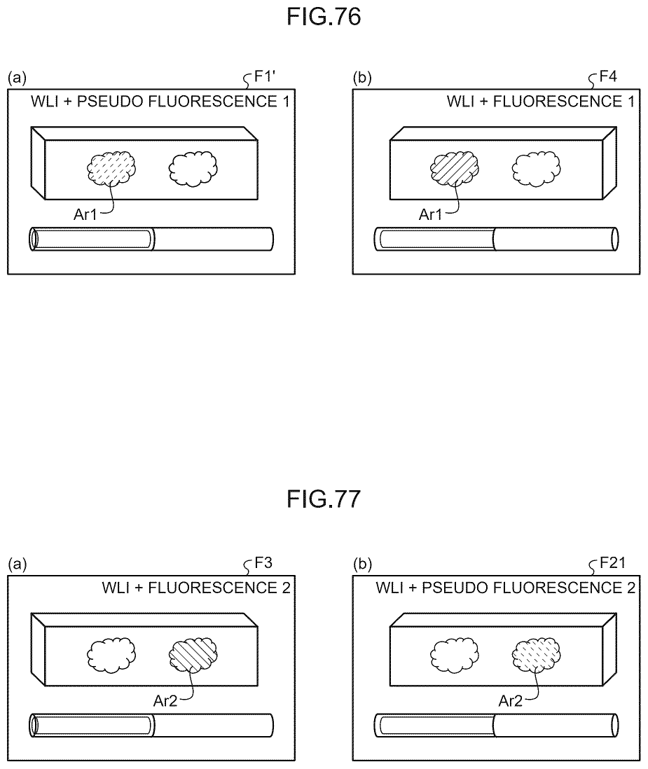

In the example of FIG. 76, the display control unit 933 displays the captured image F4 as the video signal for right eye ((b) of FIG. 76), and displays a captured image F1′ where the region representing the first fluorescence in the captured image F4 is illustrated in a pseudo manner for the captured image F1 as the video signal for left eye ((a) of FIG. 76). In (a) of FIG. 76, for convenience of description, a text “WLI+Pseudo Fluorescence 1” representing that the region representing the first fluorescence is illustrated in a pseudo manner for the captured image F1 is illustrated in the upper right of the captured image F11. In the example of FIG. 76, each of the normal observation image corresponding to the white light and the region Ar1 where the fluorescence intensity of the first fluorescence is strong is displayed in 3D.

In the example of FIG. 77, the display control unit 933 displays the captured image F3 as the video signal for left eye ((a) of FIG. 77), and displays a captured image F21 where the region representing the second fluorescence in the captured image F3 is illustrated in a pseudo manner for the captured image F2 as the video signal for right eye ((b) of FIG. 77). In (b) of FIG. 77, for convenience of description, a text “WLI+Pseudo Fluorescence 2” representing that the region representing the second fluorescence is illustrated in a pseudo manner for the captured image F2 is illustrated in the upper right of the captured image F21. In the example of FIG. 77, each of the normal observation image corresponding to the white light and the region Ar2 where the fluorescence intensity of the second fluorescence is strong is displayed in 3D.

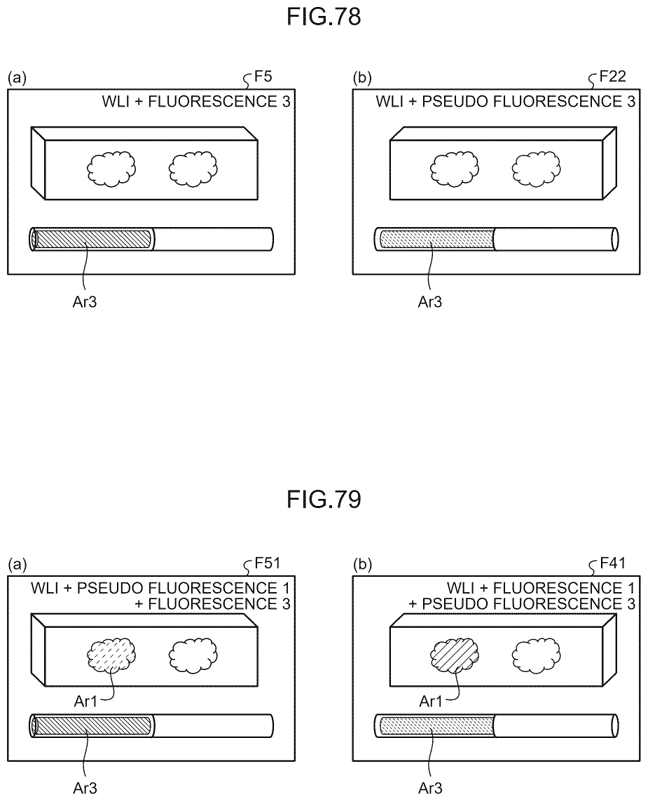

In the example of FIG. 78, the display control unit 933 displays the captured image F5 as the video signal for left eye ((a) of FIG. 78), and displays a captured image F22 where the region representing the third fluorescence in the captured image F5 is illustrated in a pseudo manner for the captured image F2 as the video signal for right eye ((b) of FIG. 78). In (b) of FIG. 78, for convenience of description, a text “WLI+Pseudo Fluorescence 3” representing that the region representing the third fluorescence is illustrated in a pseudo manner for the captured image F2 is illustrated in the upper right of the captured image F22. In the example of FIG. 78, each of the normal observation image corresponding to the white light and the region Ar3 where the fluorescence intensity of the third fluorescence is strong is displayed in 3D.

In the example of FIG. 79, the display control unit 933 displays a captured image F51 where the region representing the first fluorescence in the captured image F4 is illustrated in a pseudo manner for the captured image F5 as the video signal for left eye ((a) of FIG. 79), and displays a captured image F41 where the region representing the third fluorescence in the captured image F5 is illustrated in a pseudo manner for the captured image F4 as the video signal for right eye ((b) of FIG. 79). In (a) of FIG. 79, for convenience of description, a text “WLI+Pseudo Fluorescence 1+Fluorescence 3” representing that the region representing the first fluorescence is illustrated in a pseudo manner for the captured image F5 is illustrated in the upper right of the captured image F51. In (b) of FIG. 79, a text “WLI+Fluorescence 1+Pseudo Fluorescence 3” representing that the region representing the third fluorescence is illustrated in a pseudo manner for the captured image F4 is illustrated in the upper right of the captured image F41. In the example of FIG. 79, each of the normal observation image corresponding to the white light and the regions Ar1 and Ar3 where the fluorescence intensities of the first and third fluorescence components are strong is displayed in 3D.

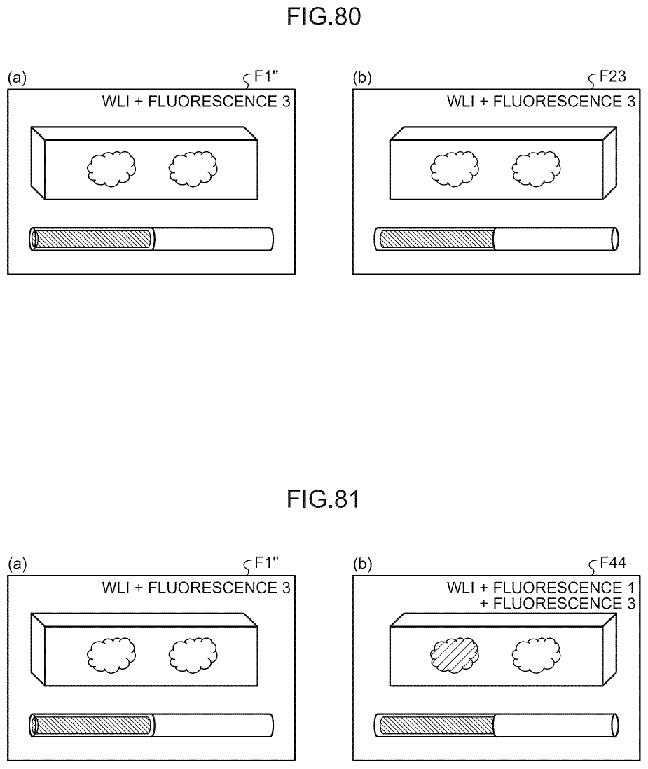

In the example of FIG. 80, the display control unit 933 displays a captured image F1″ where the third fluorescence observation image generated by the second imaging unit 52 is superimposed on the captured image F1 as the video signal for left eye ((a) of FIG. 80), and displays a captured image F23 where the third fluorescence observation image generated by the first imaging unit 51 is superimposed on the captured image F2 as the video signal for right eye ((b) of FIG. 80). That is, each of the normal observation image corresponding to the white light and the region representing the third fluorescence is displayed in 3D.

In the example of FIG. 81, the display control unit 933 displays the captured image F1″ as the video signal for left eye ((a) of FIG. 81), and displays a captured image F44 where the third fluorescence observation image generated by the first imaging unit 51 is superimposed on the captured image F4 as the video signal for right eye ((b) of FIG. 81). That is, each of the normal observation image corresponding to the white light and the region representing the third fluorescence is displayed in 3D, and the region representing the first fluorescence is displayed in 2D.

Even with the display of Modification Example 6 described above, the same effects as in the above-described embodiment are exhibited.

The display method may be a display method that varies depending on each display mode.

For example, when set to the first display mode, a display image that enables stereoscopic vision using the normal observation images for right and left eyes is generated. In addition, when set to a second display mode, one of the normal observation images for right and left eyes and at least one of information representing a fluorescent region in the first fluorescence observation image or information representing a fluorescent region in the second fluorescence observation image are two-dimensionally displayed.

A medical observation system according to Modification Example 7 is a medical observation system using a so-called video scope (flexible endoscope) including an imaging unit on a distal end side of the insertion unit. Hereinafter, for convenience of description, the medical observation system 1 according to Modification Example 1 will be referred to as a medical observation system 1B.

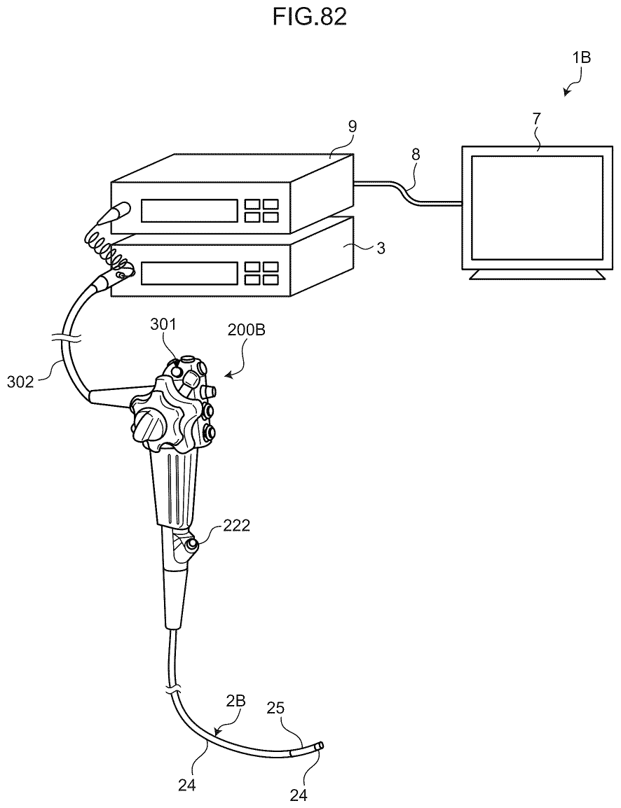

FIG. 82 is a diagram illustrating Modification Example 7 of the embodiment.

As illustrated in FIG. 82, the medical observation system 1B includes: an endoscope 300B where an insertion unit 2B is inserted into a living body such that an in-vivo image of an observed region is captured to output a captured image; the light source device 3 that emits white light and excitation light from a distal end of the endoscope 300B; the control device 9 that processes the captured image output from the endoscope 300B; and the display device 7 that is connected to the control device 9 through the second transmission cable 8 to display an image based on a video signal processed by the control device 9.

As illustrated in FIG. 82, the endoscope 300B includes: the insertion unit 2B having a flexible elongated shape; an operating unit 301 that is connected to a proximal end side of the insertion unit 2B and receives various operations; and a universal cord 302 that extends from the operating unit 301 in a direction different from a direction in which the insertion unit 2B extends and is equipped with various cables connected to the light source device 3 and the control device 9.

As illustrated in FIG. 82, the insertion unit 2B includes: a distal end portion 24; a bending portion 25 that is connected to a proximal end side of the distal end portion 24 and is configured to be bendable by a plurality of bending pieces; and an elongated flexible tube portion 26 that is connected to a proximal end side of the bending portion 25.

Although not illustrated in detail, the distal end portion 24 is equipped with substantially the same configuration as that of the camera head 5 described in the above-described embodiment. The captured image obtained by the distal end portion 24 is output to the control device 9 through the operating unit 301 and the universal cord 302.

Even when the configuration of Modification Example 7 described above is adopted, the same effects as in the above-described embodiment are exhibited.

A medical observation system according to Modification Example 8 is a medical observation system using an operating microscope that enlarges and captures a predetermined viewing region in a subject (in a living body) or on a subject surface (living body surface) that is an observation target. Hereinafter, for convenience of description, the medical observation system 1 according to Modification Example 3 will be referred to as a medical observation system 1C.

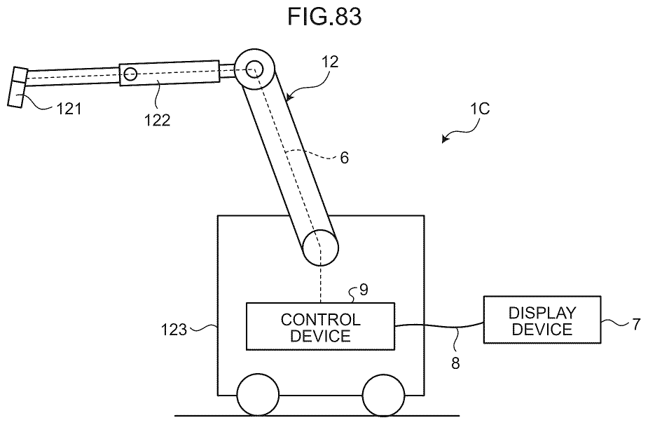

FIG. 83 is a diagram illustrating Modification Example 8 of the embodiment.

As illustrated in FIG. 83, the medical observation system 1C includes: an operating microscope 12 that captures an image for observing a subject to output a captured image; the control device 9 that processes the captured image output from the operating microscope 12; and the display device 7 that is connected to the control device 9 through the second transmission cable 8 to display an image based on a video signal processed by the control device 9.

As illustrated in FIG. 83, the operating microscope 12 includes: a microscope portion 121 that enlarges and captures a micro portion of a subject to output a captured image; a support portion 122 including an arm that is connected to a proximal end portion of the microscope portion 121 and rotatably supports the microscope portion 121; and a base portion 123 that rotatably holds a proximal end portion of the support portion 122 and is movable on a floor.

As illustrated in FIG. 83, the control device 9 is provided in the base portion 123. In addition, although not illustrated in detail, in the base portion 123, the light source device 3 that emits white light and excitation light from the operating microscope 12 to the observation target is also provided in the base portion 123.

The base portion 123 may be fixed to a ceiling or a wall surface to support the support portion 122 instead of being movably provided on the floor.

Although not illustrated in detail, the microscope portion 121 is equipped with substantially the same configuration as that of the camera head 5 described in the above-described embodiment. The captured image obtained by the microscope portion 121 is output to the control device 9 through the first transmission cable 6 that is wired along the support portion 122.

Even when the configuration of Modification Example 8 described above is adopted, the same effects as in the above-described embodiment are exhibited.

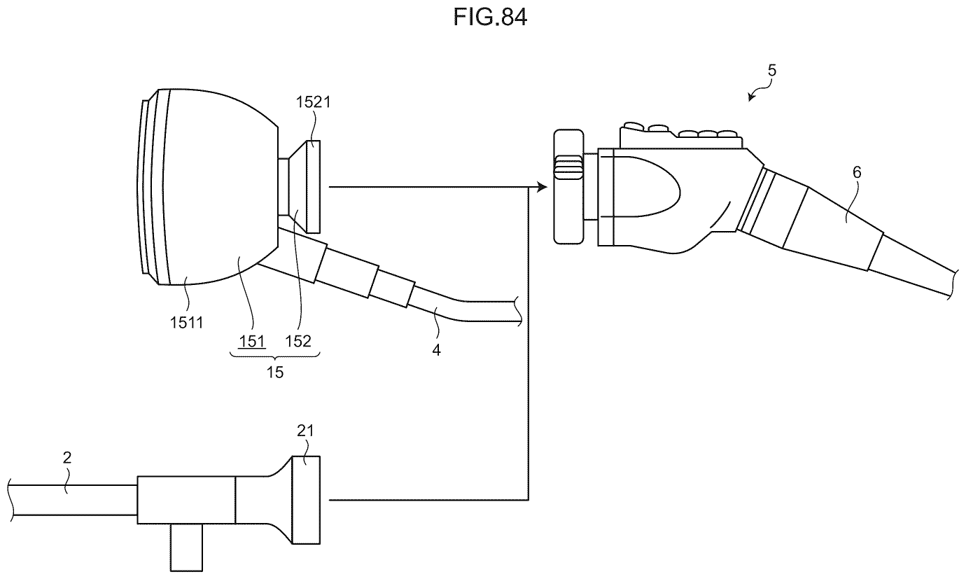

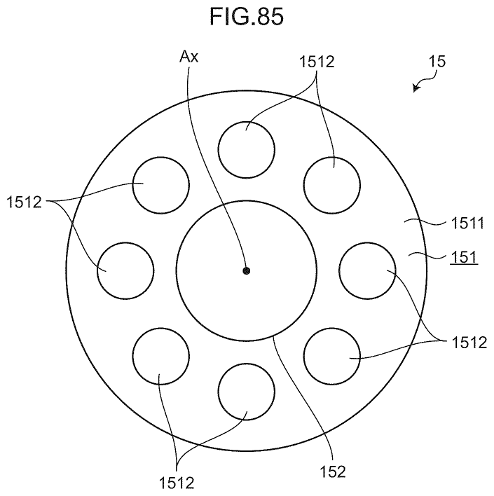

FIGS. 84 and 85 are diagrams illustrating Modification Example 9 of the embodiment. Specifically, FIG. 84 is a diagram illustrating a ringlight 15 when seen from the side. FIG. 85 is a diagram illustrating the ringlight 15 when seen from the front side (in FIG. 84, the left side).

In Modification Example 9, not only the insertion unit 2 described in the above-described embodiment but also the ringlight 15 illustrated in FIGS. 84 and 85 are detachably connected to the camera head 5. That is, depending on a usage state of a user, the insertion unit 2 or the ringlight 15 may be connected to the camera head 5 as illustrated in FIG. 84.

The ringlight 15 is not inserted into the observation target unlike the insertion unit 2, supplies first light and excitation light to an operation site, and takes in return light of the white light and the excitation light from the operation site. As illustrated in FIGS. 84 and 85, the ringlight 15 includes an illumination unit 151 and a subject image take-in unit 152 that takes in the subject image.

As illustrated in FIGS. 84 and 85, the illumination unit 151 includes a casing 1511 and a plurality of illumination lenses 1512.

The casing 1511 has an annular shape around an optical axis Ax. The other end of the light guide 4 is detachably connected to the casing 1511.

As illustrated in FIG. 85, the plurality of illumination lenses 1512 are disposed at a predetermined interval in a circumferential direction around the optical axis Ax on an end surface of the casing 1511 on the front side. Each of the plurality of illumination lenses 1512 irradiates the operation site with the white light and the excitation light that are supplied from the light source device 3 and introduced into the casing 1511 through the light guide 4.

The subject image take-in unit 152 extends along the optical axis Ax. In addition, in the subject image take-in unit 152, an optical system that is configured using one or a plurality of lenses and focuses the return light (subject image) of the white light and the excitation light emitted from the plurality of illumination lenses 1512 through the operation site is provided. In FIG. 85, the subject image take-in unit 152 is described assuming that the subject side of the objective optical system is configured to be a monocular type and an intermediate portion is configured to be a binocular type. However, a configuration where the entire portion is a binocular type may also be adopted. Further, a connection portion 1521 is provided in an end portion of the subject image take-in unit 152 on the proximal end side (in FIG. 84, the right side). The connection portion 1521 has a design (shape) that is compatible with the eyepiece unit 21 in the insertion unit 2, and is detachably connected to the camera head 5.

Even when the configuration of Modification Example 9 described above is adopted, the same effects as in the above-described embodiment are exhibited.

The following configurations also belong to the technical scope of the present disclosure.

With a medical stereoscopic observation imaging device, a medical stereoscopic observation system, and a medical image processing device according to the present disclosure, miniaturization can be achieved while enabling the normal light and two types of fluorescence to be captured.

Although the disclosure has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A medical stereoscopic observation imaging device comprising:
a first imaging unit configured to capture each of first normal observation light and at least one of first fluorescence or second fluorescence; and
a second imaging unit configured to capture each of second normal observation light and at least an other one of the first fluorescence or the second fluorescence,
wherein the first normal observation light and the second normal observation light are components of return light from an observation target with which normal light including at least a part of a wavelength band of visible light is irradiated and are components of observation light having parallax with each other, and
the first fluorescence and the second fluorescence are components of fluorescence emitted from the observation target and have different wavelength bands.

2. The medical stereoscopic observation imaging device according to claim 1, wherein
the first imaging unit includes
a first image sensor configured to capture the first normal observation light, and
a second image sensor configured to capture the first fluorescence, and
the second imaging unit includes
a third image sensor configured to capture the second normal observation light, and
a fourth image sensor configured to capture the second fluorescence.

3. The medical stereoscopic observation imaging device according to claim 2, wherein
the fourth image sensor is configured to capture each of the second fluorescence and third fluorescence,
the third fluorescence is fluorescence that is emitted from the observation target and has a stronger fluorescence intensity than the second fluorescence.

4. The medical stereoscopic observation imaging device according to claim 2, wherein
the first imaging unit is configured to capture each of the first normal observation light, the first fluorescence, and first fluorescence observation light,
the second imaging unit s configured to capture each of the second normal observation light, the second fluorescence, and second fluorescence observation light, and
the first fluorescence observation light and the second fluorescence observation light are components of third fluorescence that are emitted from the observation target and have a stronger fluorescence intensity than the second fluorescence and are components of observation light having parallax with each other.

5. The medical stereoscopic observation imaging device according to claim 4, wherein
the first image sensor is configured to capture each of the first normal observation light and the first fluorescence observation light, and
the third image sensor is configured to capture each of the second normal observation light and the second fluorescence observation light.

6. The medical stereoscopic observation imaging device according to claim 4, wherein
the first image sensor is configured to capture each of the first normal observation light and the first fluorescence observation light, and
the fourth image sensor is configured to capture each of the second fluorescence and the second fluorescence observation light.

7. The medical stereoscopic observation imaging device according to claim 1, wherein
the first imaging unit includes a first image sensor configured to capture each of the first normal observation light and the first fluorescence, and
the second imaging unit includes a second image sensor configured to capture each of the second normal observation light and the second fluorescence.

8. The medical stereoscopic observation imaging device according to claim 1, wherein
the first imaging unit includes
a first image sensor configured to capture the first normal observation light, and
a second image sensor configured to capture each of the first fluorescence and the second fluorescence, and
the second imaging unit includes a third image sensor configured to capture the second normal observation light.

9. The medical stereoscopic observation imaging device according to claim 8, wherein
the first imaging unit is configured to capture each of the first normal observation light, the first fluorescence, the second fluorescence, and first fluorescence observation light,
the second imaging unit is configured to capture each of the second normal observation light and second fluorescence observation light, and
the first fluorescence observation light and the second fluorescence observation light are components of third fluorescence that are emitted from the observation target and have a stronger fluorescence intensity than the second fluorescence and are components of observation light having parallax with each other.

10. The medical stereoscopic observation imaging device according to claim 9, wherein
the first image sensor is configured to capture each of the first normal observation light and the first fluorescence observation light, and
the third image sensor is configured to capture each of the second normal observation light and the second fluorescence observation light.

11. The medical stereoscopic observation imaging device according to claim 9, wherein
the second image sensor is configured to capture each of the first fluorescence, the second fluorescence, and the first fluorescence observation light, and
the third image sensor is configured to capture at least one of the second normal observation light, the second fluorescence, or the second fluorescence observation light.

12. A medical stereoscopic observation system comprising:
a medical stereoscopic observation imaging device including
a first imaging unit configured to capture each of first normal observation light and at least one of first fluorescence or second fluorescence, and
a second imaging unit configured to capture each of second normal observation light and at least the other one of the first fluorescence and the second fluorescence; and
a medical image processing device configured to process a captured image obtained by capturing of the medical stereoscopic observation imaging device,
wherein the first normal observation light and the second normal observation light are components of return light from an observation target with which normal light including at least a part of a wavelength band of visible light is irradiated and are components of observation light having parallax with each other, and
the first fluorescence and the second fluorescence are components of fluorescence emitted from the observation target and have different wavelength bands.

13. The medical stereoscopic observation system according to claim 12,
wherein the medical image processing device is configured to generate a display image to be displayed by a display device based on at least one of a first normal observation image that is the captured image obtained by capturing the first normal observation light or a second normal observation image that is the captured image obtained by capturing the second normal observation light, a first fluorescence observation image that is the captured image obtained by capturing the first fluorescence, and a second fluorescence observation image that is the captured image obtained by capturing the second fluorescence.

14. The medical stereoscopic observation system according to claim 13, wherein the medical image processing device is configured to generate the display image that enables stereoscopic vision using the first normal observation image and the second normal observation image and two-dimensionally displays at least one of information representing a fluorescent region in the first fluorescence observation image or information representing a fluorescent region in the second fluorescence observation image.

15. The medical stereoscopic observation system according to claim 12, wherein
the first imaging unit is configured to capture first fluorescence observation light,
the second imaging unit is configured to capture second fluorescence observation light,
the first fluorescence observation light and the second fluorescence observation light are components of third fluorescence that are emitted from the observation target and have a stronger fluorescence intensity than the second fluorescence and are components of observation light having parallax with each other, and
the medical image processing device is configured to use a first stereoscopic fluorescence observation image that is the captured image obtained by capturing the first fluorescence observation light and a second stereoscopic fluorescence observation image that is the captured image obtained by capturing the second fluorescence observation light to generate the display image that enables stereoscopic vision of information representing a fluorescent region in the first stereoscopic fluorescence observation image and the second stereoscopic fluorescence observation image.

16. The medical stereoscopic observation system according to claim 13,
wherein when set to a first display mode, the medical image processing device is configured to generate the display image that enables stereoscopic vision using the first normal observation image and the second normal observation image, and
when set to a second display mode, the medical image processing device is configured to generate the display image that two-dimensionally displays one of the first normal observation image and the second normal observation image and at least one of information representing a fluorescent region in the first fluorescence observation image or information representing a fluorescent region in the second fluorescence observation image.

17. The medical stereoscopic observation system according to claim 13, wherein
the display image includes
a first display image that is displayed on a first screen and
a second display image that is displayed on a second screen,
the first display image enables stereoscopic vision using the first normal observation image and the second normal observation image, and
the second display image two-dimensionally displays at least one of information representing a fluorescent region in the first fluorescence observation image or information representing a fluorescent region in the second fluorescence observation image.

18. The medical stereoscopic observation system according to claim 13, wherein
the display image includes
a first display image that is displayed on a first screen and
a second display image that is displayed on a second screen,
the first display image enables stereoscopic vision using the first normal observation image and the second normal observation image, and
the second display image two-dimensionally displays one of the first normal observation image and the second normal observation image and at least one of information representing a fluorescent region in the first fluorescence observation image or information representing a fluorescent region in the second fluorescence observation image.

19. The medical stereoscopic observation system according to claim 13, wherein
the display image includes
a first display image that is displayed on a first screen and
a second display image that is displayed on a second screen,
the first display image enables stereoscopic vision using the first normal observation image and the second normal observation image, and
the second display image enables stereoscopic vision using the first normal observation image and the second normal observation image and two-dimensionally displays at least one of information representing a fluorescent region in the first fluorescence observation image or information representing a fluorescent region in the second fluorescence observation image.

20. A medical image processing device comprising
a processor configured to process a captured image obtained by capturing of a medical stereoscopic observation imaging device, wherein
the medical stereoscopic observation imaging device includes:
a first imaging unit configured to capture each of first normal observation light and at least one of first fluorescence or second fluorescence; and
a second imaging unit configured to capture each of second normal observation light and at least the other one of the first fluorescence and the second fluorescence,
the first normal observation light and the second normal observation light are components of return light from an observation target with which normal light including at least a part of a wavelength band of visible light is irradiated and are components of observation light having parallax with each other, and
the first fluorescence and the second fluorescence are components of fluorescence emitted from the observation target and have different wavelength bands.
